(19) United States
(12) Patent Application Publication
(10) Pub. No.: US 2025/0127694 A1
(43) Pub. Date: Apr. 24, 2025

(54) FRAGRANCE COMPOSITIONS AND USES THEREOF

(71) Applicant: Coty Inc., New York, NY (US)

(72) Inventors: Aurélien Saint-Paul, Sergy (FR); Enrica Perrotta, Geneva (CH); Jose Maria Velazquez Mendoza, Nyon (CH); Sarah Kyakyo Kanyunyuzi Nyakana, Versoix (CH)

(21) Appl. No.: 18/696,049

(22) PCT Filed: Sep. 29, 2022
(86) PCT No.: PCT/US2022/045220

§ 371 Date: Mar. 27, 2024

Related U.S. Application Data
(60) Provisional application No. 63/250,907, filed on Sep. 30, 2021.

(51) Int. Cl. A61K 8/34; A61Q 13/00

(52) U.S. Cl. CPC A61K 8/345; A61Q 13/00

(57) ABSTRACT

According to various examples, a fragrance composition can include a modulator component in a range of from about 0.1 wt % to about 30 wt % of the fragrance composition. A fragrance component is present in an amount in a range of from about 0.04 wt % to about 40 wt % of the fragrance composition, the fragrance component includes at least one low volatile fragrance material having a vapor pressure less than 0.001 Torr (0.000133 kPa) at 25° C.; at least one moderate volatile fragrance material having a vapor pressure in the range of 0.1 Torr to 0.001 Torr (0.0133 kPa to 0.000133 kPa) at 25° C.; and at least one high volatile fragrance material having a vapor pressure greater than 0.1 Torr (0.0133 kPa) at 25° C.

### CROSS REFERENCE TO RELATED APPLICATIONS

This application claims priority to U.S. Provisional Patent Application No. 63/250,907, titled FRAGRANCE COMPOSITIONS AND USES THEREOF, and filed on Sep. 30, 2021, the contents of which are hereby incorporated by reference.

### FIELD OF THE INVENTION

Inventive subject matter herein relates to the field of perfumery. In particular, it provides compositions comprising fragrance materials and at least one substantially fragrance modulator for improving or enhancing the fidelity and/or longevity of the fragrance profile. The invention also relates to methods of making and using said compositions.

### BACKGROUND OF THE DISCLOSURE

Fragrances can include fragrance components that can be classified, in part, by their volatility. Accordingly these fragrance components may be referred to as a high-, moderate-, or low-volatility fragrance components. Different fragrances may be dominated by any one or more of these component such that the fragrance may be associated with different perceptions by a user. While some high- or moderate-volatility fragrances may be associated with a favorable perception, the volatility of the fragrance may result in a short timeframe in which the fragrance is perceived by a panel of experts or professional evaluators or individual experts or professional evaluators or in a rapid loss of the initial character of the fragrance (e.g., citrus, aquatic, aromatic, floral, spicy, fresh, or a combination thereof) and becoming dominated by a heavy fragrance character (e.g., woody or musky).

### SUMMARY OF THE DISCLOSURE

According to various examples, a fragrance composition can include a modulator component in a range of from about 0.1 wt % to about 30 wt % of the fragrance composition. A fragrance component is present in an amount in a range of from about 0.04 wt % to about 40 wt % of the fragrance composition, the fragrance component includes at least one low volatile fragrance material having a vapor pressure less than 0.001 Torr (0.000133 kPa) at 25° C.; at least one moderate volatile fragrance material having a vapor pressure in the range of 0.1 Torr to 0.001 Torr (0.0133 kPa to 0.000133 kPa) at 25° C.; and at least one high volatile fragrance material having a vapor pressure greater than 0.1 Torr (0.0133 kPa) at 25° C.

There are many non-limiting reasons for using the compositions of the instant disclosure. For example, according to various embodiments, the characteristics of the composition can provide rules for objectively classifying fragrance materials according to their volatility using their vapor pressures defined at suitable temperature, instead of their characters. The objective rules operate irrespective of perfumers performing the classification. In particular, the rules classify the fragrance materials into low, moderate or high volatile fragrance materials for formulating into fragrance mixtures. Furthermore, according to some embodiments, the compositions can have improved fidelity to the perceived fragrance profile over time. According to some embodiments, pairing the fragrance components with a selective modulator can help to ensure that an initial fragrance impression is significantly consistent from its initial impression to the end. For example, an initial impression of the fragrance can maintain its quality, as perceived by a panel of experts or professional evaluators or individual experts or professional evaluators, for a longer period of time relative to a corresponding fragrance that is free of any one of the modulators, or combinations thereof described herein or relative to the equivalent fragrance that has a traditional fragrance construction that includes a different modulator.

Beyond extending the initial impression of the fragrance, the strength and longevity of the high volatile fragrances can be improved. For example, according to some embodiments, compositions having improved longevity of the perceived fragrance profile can be present for long periods of time (e.g., greater than 30 mins, 1, 2, 4, 6, or even 8 hours). The improved longevity of the high volatile fragrances may result from the modulator slowing the evaporation of the high and moderate volatile fragrances from the composition.

Additionally, according to some embodiments, the perceived harshness of overdosing of the fragrance material is mitigated or absent, as compared to the same perception in a fragrance in the absence of the modulator.

### DETAILED DESCRIPTION OF THE INVENTION

### Definitions

As used herein, articles such as “a” and “an” when used in a claim, are understood to mean one or more of what is claimed or described.

As used herein, the terms “include”, “includes” and “including” are meant to be non-limiting.

As used herein, the term “body splash” means a body care formulation that is applied to the body. Typically, the body splash is applied to the body after bathing and provides a subtle hint of scent to the body. Body splashes are commonly used by consumers who prefer less strong fragrance compositions. A body splash may comprise an ethanol-free composition according to the present invention which comprises from 0.2-8 wt %, relative to the total weight of the composition, of a fragrance component. The body splash may further comprise alkyl polyglucosides as non-ionic surfactants.

As used herein, the term “body spray” means a formulation comprising fragrance materials intended to be applied to the body to prevent or mask body odor caused by the bacterial breakdown of perspiration on the body (e.g., armpits, feet, and other areas of the body). The body spray may also provide a fragrance expression to the a panel of experts or professional evaluators or individual experts or professional evaluators. Typically, body spray compositions are applied as an aerosol spray in an effective amount on the skin of a consumer.

As used herein, the term “composition” includes a fine fragrance composition intended for application to a body surface, such as for example, skin or hair, e.g., to impart a pleasant odor thereto, or cover a malodour thereof. They are generally in the form of perfume concentrates, perfumes, eau de parfums, eau de toilettes, aftershaves, or colognes. The fine fragrance compositions may be an ethanol-based composition. The term “composition” may also include a cosmetic composition, which comprises a fragrance material for the purposes of delivering a pleasant smell to drive consumer acceptance of the cosmetic composition. The term “composition” may also include body splashes or body sprays. The term “composition” may also include cleaning compositions, such as fabric care composition or home care compositions, including air care compositions (e.g., air fresheners), for use on clothing or other substrates such as hard surfaces (e.g., dishes, floors, countertops). Additional non-limiting examples of “composition” may also include facial or body powder, deodorant, foundation, body/facial oil, mousse, creams (e.g., cold creams), waxes, sunscreens and blocks, bath and shower gels, lip balms, self-tanning compositions, masks and patches.

As used herein, the term “consumer” means both the user of the composition and the observer nearby or around the user.

As used herein, the term “fragrance material” and “fragrance materials” relates to a perfume raw material (“PRM”), or a mixture of perfume raw materials (“PRMs”), that are used to impart an overall pleasant odor or fragrance profile to a composition. “Fragrance materials” can encompass any suitable perfume raw materials for fragrance uses, including materials such as, for example, alcohols, aldehydes, ketones, esters, ethers, acetates, nitriles, terpene hydrocarbons, nitrogenous or sulfurous heterocyclic compounds and essential oils. However, naturally occurring plant and animal oils and exudates comprising complex mixtures of various chemical components are also known for use as “fragrance materials”. The individual perfume raw materials which comprise a known natural oil can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, New Jersey, USA and more recently re-published by Allured Publishing Corporation Illinois (1994). Additionally, some perfume raw materials are supplied by the fragrance houses (Firmenich, International Flavors & Fragrances, Givaudan, Symrise) as mixtures in the form of proprietary specialty accords. Non-limiting examples of the fragrance materials useful herein include pro-fragrances such as acetal pro-fragrances, ketal pro-fragrances, ester pro-fragrances, hydrolyzable inorganic-organic pro-fragrances, and mixtures thereof. The fragrance materials may be released from the pro-fragrances in a number of ways. For example, the fragrance may be released as a result of simple hydrolysis, or by a shift in an equilibrium reaction, or by a pH-change, or by enzymatic release.

As used herein, the term “fragrance profile” means the description of how the fragrance is perceived by the human nose at any moment in time. The fragrance profile may change over time. It is a result of the combination of the low, moderate and high volatile fragrance materials, if present, of a fragrance. A fragrance profile is composed of 2 characteristics: ‘intensity’ and ‘character’. The ‘intensity’ relates to the perceived strength whilst ‘character’ refers to the odor impression or quality of the perfume, e.g., fruity, floral, woody, etc.

As used herein, the terms “modulator”, and “fragrance modulator” are used interchangeably to designate an agent having the capacity to affect the fragrance profile, such as for example, by impacting the fragrance materials' evaporation rate. The modulator may mediate its effect by lowering the vapor pressure of the fragrance materials and increasing their adherence to the substrate (skin and/or hair) thus ensuring a longer-lasting impression of the fragrance. By incorporating the modulator, it is desired that the fragrance profile, preferably the fragrance components composition attributable to the high and moderate volatile fragrance materials, alone or individually, of the composition can be perceived by a panel of experts or professional evaluators or individual experts or professional evaluators, over a longer period of time, or the perceived harshness of overdosing of the fragrance material is mitigated or absent, as compared to the same perception in the absence of the modulator. As used herein “overdose” can include overdosing a moderate volatile component or high volatile component in aggregate (e.g., greater than 30 wt % of the fragrance component). The term “overdose” can further include overdosing an individual component of the moderate volatile component or the high volatile component (e.g., if the high volatile component includes three oils at least one of the oils may account for a greater wt % of the high volatile component than would be present in a traditional fragrance or a fragrance that is free of the modulators described herein). Suitable examples of the modulator are provided herein below.

As used herein, the term “substantially non-odorous” means an agent that does not impart an odor of its own when added into a composition of the present invention. For example, a “substantially non-odorous fragrance modulator” does not impart a new odor that alters the character of the fragrance profile of the composition to which it is added. The term “substantially non-odorous” also encompasses an agent that may impart a minimal or slight odor of its own when added into a composition of the present invention. However, the odor imparted by the “substantially non-odorous fragrance modulator” is generally undetectable or tends to not substantively alter the character of the fragrance profile of the composition to which it is added initially or preferably over time. Furthermore, the term “substantially non-odorous” also includes materials that are perceivable only by a minority of people or those materials deemed “anosmic” to the majority of people. Furthermore, the term “substantially non-odorous” also includes materials that may, from particular suppliers, contain an odor due to impurities, such as when the materials contain the impurities at not more than about 5 wt %, preferably not more than 1 wt %, often even not more than 1 part per million (ppm). These impurities may be removed by purification techniques known in the art as required to make them suitable for use in fragrance compositions of the present invention.

As used herein, the term “vapor pressure” means the partial pressure in air at a defined temperature (e.g., 25° C.) and standard atmospheric pressure (760 mmHg) for a given chemical species. It defines a chemical species' desire to be in the gas phase rather than the liquid or solid state. The higher the vapor pressure the greater the proportion of the material that will, at equilibrium, be found in a closed headspace. It is also related to the rate of evaporation of a fragrance material which is defined in an open environment where material is leaving the system. The vapor pressure is determined according to the reference program Advanced Chemistry Development (ACD/Labs) Software Version 14.02, or preferably the latest version update).

It is understood that the test methods that are disclosed in the Test Methods Section of the present application must be used to determine the respective values of the parameters of Applicants' inventions as described and claimed herein.

In all embodiments of the present invention, all percentages are by weight of the total composition, as evident by the context, unless specifically stated otherwise. All ratios are weight ratios, unless specifically stated otherwise, and all measurements are made at 25° C., unless otherwise designated.

### Compositions

The inventive subject matter herein is directed towards fragrance compositions or mixtures having improved fragrance profile fidelity and longevity. Disclosed fragrance compositions can include at least a fragrance component and modulator. The fragrance component can include a wide variety of fragrance materials. The fragrance materials can be grouped in terms of their volatility. Generally, the materials can be grouped as low volatile fragrance materials, moderate volatile fragrance materials, and high volatile fragrance materials. Each group of materials can be associated with various perceptions by a panel of experts or professional evaluators or individual experts or professional evaluators. While not so limited, a high volatile fragrance may be associated with a citrus character; a moderate volatile fragrance may be associated with a spicy character; and a low volatile fragrance may be associated with a woody character. Each group of fragrance materials can include synthetic materials or natural materials. The volatility of the fragrance materials can be in reference to an individual fragrance material. Alternatively, in cases where a combination of materials produce a fragrance, for example a natural oil, the volatility may be in reference to that aggregation.

In some examples, this disclosure shows that longer lasting fragrance profiles or at least initial fragrance profiles, may be enhanced through the presence of certain modulators.

With respect to the composition, the fragrance component can be present in an amount of from about 0.04 wt % to 40 wt %, 1 wt % to about 30 wt %, about 5 wt % to about 30 wt %, or less than, equal to, or greater than about 0.04 wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, or about 40 wt % relative to the composition.

Additionally with respect to the composition, the modulator can be present in an amount of from about 0.1 wt % to about 30 wt %, about 0.1 wt % to about 27 wt %, about 0.5 wt % to about 20 wt %, or less than, equal to, or greater than about 0.1 wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or about 20 wt % relative to the composition.

As described herein, the “fragrance materials” have been classified as low, moderate or high volatile fragrance materials according to their volatility by their vapor pressure. This method of classifying fragrance materials by their vapor pressure avoids the problem of different classifications for the same fragrance material according to the traditional approach that relies on their subjective characteristic character. In the case that the fragrance materials are a natural oil, extract or absolute, which comprises a mixture of several compounds, the vapor pressure of the complete oil should be treated a mixture of the individual perfume raw material components using the reference program cited above. The individual components and their level, in any given natural oil or extract, can be determined by direct injection of the oil into a GC-MS column for analysis as known by one skilled in the art. In the scenario that the fragrance materials are a proprietary specialty accord, so called ‘bases’, the vapor pressure, using the reference program cited above, should preferably be obtained from the supplier. However, it is understood by one skilled in the art that they can physically analyze the composition of a full fragrance oil available commercially to identity the fragrance raw materials and their levels using standard GC-MS techniques. This would be irrespective of whether they had been added to the fragrance oil as individual chemicals, as components of naturals or from proprietary bases. Although proprietary bases and naturals are included in our examples, when analyzing a commercially available fragrance via GC-MS one could simply identify the components of the base or natural oil as part of the overall fragrance mixture and their levels, without being able to identify which proprietary base or natural oil the fragrance had come from.

A pH of the composition can be in a range of from about 4 to about 8, about 5 to about 7, less than, equal to, or greater than about 4, 5, 6, 7, or about 8. The ethanol concentration in any of the compositions described herein can be less than about 78 wt % ethanol, less than about 70 wt % ethanol, less than about 60 wt % ethanol, less than about 50 wt % ethanol, less than about 40 wt % ethanol, less than about 30 wt % ethanol, less than about 20 wt % ethanol, less than about 10 wt % ethanol, or free of ethanol.

### (i) Low Volatile Fragrance Materials

The fragrance component comprises at least one low volatile fragrance material. Individual low volatile fragrance materials or aggregate low volatile fragrance materials are those having a vapor pressure less than 0.001 Torr (0.000133 kPa) at 25° C. According to some examples, the composition can include at least 3 low volatile fragrance materials, or at least 4 low volatile fragrance materials, or at least 5 low volatile fragrance materials, or at least 7 low volatile fragrance materials.

If there are more than one low volatile fragrance materials, then the ranges provided hereinabove cover the total of all the low volatile fragrance materials. Examples of suitable low volatile fragrances materials are provided in Table 1A and 1B below.

Preferably, the low volatile fragrance material is selected from at least 1 material, or at least 2 materials, or at least 3 materials, or at least 5 materials, at least 7, at least 8, at least 10, or at least 12 low volatile fragrance materials as disclosed in Table 1A. Natural fragrance materials or oils having an aggregate vapour pressure less than 0.001 Torr (0.000133 kPa) at 25° C. are provided in Table 1B. Low Volatile Natural Oils.

TABLE 1A
Low Volatile Fragrance Materials

| | | | | Vapor |
|---|---|---|---|---|
| | | | | Pressure |
| | CAS | IUPAC | Common | (Torr at |
| No. | Number | Name | Name** | 25° C.)* |
| 1. | 1211-29-6 | Cyclopentaneacetic | Methyl | 0.00096500 |
| | | acid, 3-oxo-2-(2Z)- | jasmonate | |
| | | 2-penten-1-yl-, | | |
| | | methyl ester, | | |
| | | (1R,2R)- | | |
| 2. | 28219-60-5 | 2-Buten-1-ol, | Hindinol | 0.00096100 |
| | | 2-methyl-4- | | |
| | | (2,2,3-trimethyl-3- | | |
| | | cyclopenten-1-yl)- | | |
| 3. | 93-08-3 | Ethanone, 1-(2- | Methyl | 0.00095700 |
| | | naphthalenyl)- | beta- | |
| | | | naphthyl | |
| | | | ketone | |
| 4. | 67633-95-8 | 3-Decanone, | Methyl | 0.00095100 |
| | | 1-hydroxy- | | |
| 5. | 198404-98-7 | Cyclopropanemethanol, | Javanol ® | 0.00090200 |
| | | 1-methyl-2-[(1,2,2- | | |
| | | trimethylbicyclo[3.1.0] | | |
| | | hex-3-yl)methyl]- | | |
| 6. | 121-32-4 | Benzaldehyde, | Ethyl | 0.00088400 |
| | | 3-ethoxy-4-hydroxy- | vanillin | |
| 7. | 72403-67-9 | 3-Cyclohexene-1- | Myraldyl- | 0.00087900 |
| | | methanol, 4-(4-methyl- | acetate | |
| | | 3-penten-1-yl)-, | | |
| | | 1-acetate | | |
| 8. | 28940-11-6 | 2H-1,5-Benzodioxepin- | Calone | 0.00083100 |
| | | 3(4H)-one, 7-methyl- | | |
| 9. | 139504-68-0 | 2-Butanol, 1-[[2-(1,1- | Amber core | 0.00080300 |
| | | dimethylethyl) | | |
| | | cyclohexyl]oxy]- | | |
| 10. | 502847-01-0 | Spiro[5.5]undec-8-en- | Spiro[5.5] | 0.00073100 |
| | | 1-one, 2,2,7,9- | undec-8-en- | |
| | | tetramethyl- | 1-one, | |
| | | | 2,2,7,9- | |
| | | | tetramethyl- | |
| 11. | 2570-03-8 | Cyclopentaneacetic | trans-Hedione | 0.00071000 |
| | | acid, 3-oxo-2-pentyl-, | | |
| | | methyl ester, (1R,2R)- | | |
| | | rel- | | |
| 12. | 24851-98-7 | Cyclopentaneacetic | Methyl | 0.00071000 |
| | | acid, 3-oxo-2-pentyl-, | dihydrojasmonate | |
| | | methyl ester | or alternatives ¹ | |
| 13. | 101-86-0 | Octanal, 2- | Hexyl cinnamic | 0.00069700 |
| | | (phenylmethylene)- | aldehyde | |
| 14. | 365411-50-3 | Indeno[4,5-d]-1,3- | Nebulone | 0.00069200 |
| | | dioxin, 4,4a,5,6,7,8,9,9b- | | |
| | | octahydro-7,7,8,9,9- | | |
| | | pentamethyl- | | |
| 15. | 37172-53-5 | Cyclopentanecarboxylic | Dihydro Iso | 0.00067500 |
| | | acid, 2-hexyl-3-oxo-, | Jasmonate | |
| | | methyl ester | | |
| 16. | 65113-99-7 | 3-Cyclopentene-1- | Sandalore ® | 0.00062500 |
| | | butanol, α,β,2,2,3- | | |
| | | pentamethyl- | | |
| 17. | 68133-79-9 | Cyclopentanone, 2-(3,7- | Apritone | 0.00062000 |
| | | dimethyl-2,6-octadien- | | |
| | | 1-yl)- | | |
| 18. | 7212-44-4 | 1,6,10-Dodecatrien-3-ol, | Nerolidol | 0.00061600 |
| | | 3,7,11-trimethyl- | | |
| 19. | 53243-59-7 | 2-Pentenenitrile, 3- | Citronitril | 0.00061500 |
| | | methyl-5-phenyl-, (2Z)- | | |
| 20. | 134123-93-6 | Benzenepropanenitrile, | Fleuranil | 0.00057600 |
| | | 4-ethyl-α,α-dimethyl- | | |
| 21. | 77-53-2 | 1H-3a,7-Methanoazulen- | Cedrol Crude | 0.00056900 |
| | | 6-ol, octahydro-3,6,8,8- | | |
| | | tetramethyl-, | | |
| | | (3R,3aS,6R,7R,8aS)- | | |
| 22. | 68155-66-8 | Ethanone, 1- | Iso Gamma | 0.00056500 |
| | | (1,2,3,5,6,7,8,8a- | Super | |
| | | octahydro-2,3,8,8- | | |
| | | tetramethyl-2- | | |
| | | naphthalenyl)- | | |
| 23. | 54464-57-2 | Ethanone, 1- | Iso-E Super ® | 0.00053800 |
| | | (1,2,3,4,5,6,7,8- | | |
| | | octahydro-2,3,8,8- | | |
| | | tetramethyl-2- | | |
| | | naphthalenyl)- | | |
| 24. | 774-55-0 | Ethanone, 1-(5,6,7,8- | Florantone | 0.00053000 |
| | | tetrahydro-2- | | |
| | | naphthalenyl)- | | |
| 25. | 141-92-4 | 2-Octanol, 8,8- | Hydroxycitronellal | 0.00052000 |
| | | dimethoxy-2,6- | Dimethyl | |
| | | dimethyl- | Acetal | |
| 26. | 20665-85-4 | Propanoic acid, 2- | Vanillin | 0.00051200 |
| | | methyl-, 4-formyl-2- | isobutyrate | |
| | | methoxyphenyl ester | | |
| 27. | 79-78-7 | 1,6-Heptadien-3-one, | Hexalon | 0.00049800 |
| | | 1-(2,6,6-trimethyl-2- | | |
| | | cyclohexen-1-yl)- | | |
| 28. | 6259-76-3 | Benzoic acid, 2- | Hexyl | 0.00049100 |
| | | hydroxy-, hexyl ester | Salicylate | |
| 29. | 93-99-2 | Benzoic acid, phenyl | Phenyl Benzoate | 0.00047900 |
| | | ester | | |
| 30. | 153859-23-5 | Cyclohexanepropanol, | Norlimbanol | 0.00046900 |
| | | 2,2,6-trimethyl-α-propyl-, | | |
| | | (1R,6S)- | | |
| 31. | 70788-30-6 | Cyclohexanepropanol, | Timberol/ | 0.00046900 |
| | | 2,2,6-trimethyl-α-propyl- | Norlimbanol | |
| 32. | 68555-58-8 | Benzoic acid, 2-hydroxy-, | Prenyl Salicylate | 0.00045700 |
| | | 3-methyl-2-buten-1-yl | | |
| | | ester | | |
| 33. | 950919-28-5 | 2H-1,5-Benzodioxepin- | Cascalone | 0.00045500 |
| | | 3(4H)-one, 7-(1- | | |
| | | methylethyl)- | | |
| 34. | 30168-23-1 | Butanal, 4-(octahydro- | Dupical | 0.00044100 |
| | | 4,7-methano-5H-inden- | | |
| | | 5-ylidene)- | | |
| 35. | 1222-05-5 | Cyclopenta[g]-2- | Galaxolide ® | 0.00041400 |
| | | benzopyran, 1,3,4,6,7,8- | | |
| | | hexahydro-4,6,6,7,8,8- | | |
| | | hexamethyl- | | |
| 36. | 4602-84-0 | 2,6,10-Dodecatrien-1-ol, | Farnesol | 0.00037000 |
| | | 3,7,11-trimethyl- | | |
| 37. | 95962-14-4 | Cyclopentanone, 2-[2-(4- | Nectaryl | 0.00036700 |
| | | methyl-3-cyclohexen-1- | | |
| | | yl)propyl]- | | |
| 38. | 4674-50-4 | 2(3H)-Naphthalenone, | Nootkatone | 0.00035800 |
| | | 4,4a,5,6,7,8-hexahydro- | | |
| | | 4,4a-dimethyl-6-(1- | | |
| | | methylethenyl)-, | | |
| | | (4R,4aS,6R)- | | |
| 39. | 3487-99-8 | 2-Propenoic acid, | Amyl Cinnamate | 0.00035200 |
| | | 3-phenyl-, pentyl ester | | |
| 40. | 10522-41-5 | 2-hydroxy-2-phenylethyl | Styrolyl Acetate | 0.00033900 |
| | | acetate | | |
| 41. | 118-71-8 | 4H-Pyran-4-one, 3- | Maltol | 0.00033700 |
| | | hydroxy-2-methyl- | | |
| 42. | 128119-70-0 | 1-Propanol, 2-methyl-3- | Bornafix | 0.00033400 |
| | | [(1,7,7-trimethylbicyclo | | |
| | | [2.2.1]hept-2-yl)oxy]- | | |
| 43. | 103614-86-4 | 1-Naphthalenol, | Octalynol | 0.00033200 |
| | | 1,2,3,4,4a,5,8,8a- | | |
| | | octahydro-2,2,6,8- | | |
| | | tetramethyl- | | |
| 44. | 7785-33-3 | 2-Butenoic acid, | Geranyl Tiglate | 0.00033200 |
| | | 2-methyl-, (2E)-3,7- | | |
| | | dimethyl-2,6-octadien-1- | | |
| | | yl ester, (2E)- | | |
| 45. | 117933-89-8 | 1,3-Dioxane, 2-(2,4- | Karanal | 0.00033100 |
| | | dimethyl-3-cyclohexen- | | |
| | | 1-yl)-5-methyl-5-(1- | | |
| | | methylpropyl)- | | |
| 46. | 629-92-5 | Nonadecane | Nonadecane | 0.00032500 |
| 47. | 67801-20-1 | 4-Penten-2-ol, 3-methyl- | Ebanol | 0.00028100 |
| | | 5-(2,2,3-trimethyl-3- | | |
| | | cyclopenten-1-yl)- | | |
| 48. | 65416-14-0 | Propanoic acid, 2- | Maltol Isobutyrate | 0.00028000 |
| | | methyl-, 2-methyl-4-oxo- | | |
| | | 4H-pyran-3-yl ester | | |
| 49. | 28219-61-6 | 2-Buten-1-ol, 2-ethyl-4- | Laevo Trisandol | 0.00028000 |
| | | (2,2,3-trimethyl-3- | | |
| | | cyclopenten-1-yl)- | | |
| 50. | 5986-55-0 | 1,6-Methanonaphthalen- | Healingwood | 0.00027800 |
| | | 1(2H)-ol, octahydro- | | |
| | | 4,8a,9,9-tetramethyl-, | | |
| | | (1R,4S,4aS,6R,8aS)- | | |
| 51. | 195251-91-3 | 2H-1,5-Benzodioxepin- | Transluzone | 0.00026500 |
| | | 3(4H)-one, 7-(1,1- | | |
| | | dimethylethyl)- | | |
| 52. | 3100-36-5 | 8-Cyclohexadecan-1-one | Cyclohexadecenone | 0.00025300 |
| 53. | 65405-77-8 | Benzoic acid, 2-hydroxy-, | cis-3-Hexenyl | 0.00024600 |
| | | (3Z)-3-hexen-1-yl ester | salicylate | |
| 54. | 4940-11-8 | 4H-Pyran-4-one, 2-ethyl- | Ethyl Maltol | 0.00022800 |
| | | 3-hydroxy- | | |
| 55. | 541-91-3 | Cyclopentadecanone, | Muskone | 0.00017600 |
| | | 3-methyl- | | |
| 56. | 118-58-1 | Benzoic acid, 2-hydroxy-, | Benzyl salicylate | 0.00017500 |
| | | phenylmethyl ester | | |
| 57. | 81783-01-9 | 6,8-Nonadien-3-one, | Labienoxime | 0.00017300 |
| | | 2,4,4,7-tetramethyl-, | | |
| | | oxime | | |
| 58. | 25485-88-5 | Benzoic acid, 2-hydroxy-, | Cyclohexyl | 0.00017300 |
| | | cyclohexyl ester | | |
| 59. | 91-87-2 | Benzene, [2- | Amyl Cinnamic | 0.00016300 |
| | | (dimethoxymethyl)-1- | Aldehyde Dimethyl | |
| | | hepten-1-yl]- | Acetal | |
| 60. | 104864-90-6 | 3-Cyclopentene-1- | Firsantol | 0.00016000 |
| | | butanol, β,2,2,3- | | |
| | | tetramethyl-δ-methylene- | | |
| 61. | 224031-70-3 | 4-Penten-1-one, 1-spiro | Spirogalbanone | 0.00015300 |
| | | [4.5]dec-7-en-7-yl- | | |
| 62. | 134-28-1 | 5-Azulenemethanol, | Guaiyl Acetate | 0.00013400 |
| | | 1,2,3,4,5,6,7,8- | | |
| | | octahydro-α,α-3,8- | | |
| | | tetramethyl-, 5-acetate, | | |
| | | (3S,5R,8S)- | | |
| 63. | 236391-76-7 | Acetic acid, 2-(1- | Romandolide ® | 0.00012400 |
| | | oxopropoxy)-, 1-(3,3- | | |
| | | dimethylcyclohexyl) | | |
| | | ethyl ester | | |
| 64. | 115-71-9 | 2-Penten-1-ol, | cis-alpha-Santalol | 0.00011800 |
| | | 5-[(1R,3R,6S)-2,3- | | |
| | | dimethyltricyclo | | |
| | | [2.2.1.02,6]hept-3-yl]- | | |
| | | 2-methyl-, (2Z)- | | |
| 65. | 107898-54-4 | 4-Penten-2-ol, 3,3,- | Polysantol ® | 0.00011700 |
| | | dimethyl-5-(2,2,3- | | |
| | | trimethyl-3-cyclopenten- | | |
| | | 1-yl)- | | |
| 66. | 69486-14-2 | 5,8-Methano-2H-1- | Florex ® | 0.00011000 |
| | | benzopyran-2-ol, 6- | | |
| | | ethylideneoctahydro- | | |
| 67. | 84697-09-6 | Heptanal, 2-[(4- | Acalea | 0.00010100 |
| | | methylphenyl)methylene]- | | |
| 68. | 14595-54-1 | 4-Cyclopentadecen-1- | Exaltenone | 0.00009640 |
| | | one, (4Z)- | | |
| 69. | 32388-55-9 | Ethanone, 1-[(3R,3aR,7R, | Vertofix ® | 0.00008490 |
| | | 8aS)-2,3,4,7,8,8a- | | |
| | | hexahydro-3,6,8,8- | | |
| | | tetramethyl-1H-3a,7- | | |
| | | methanoazulen-5-yl]- | | |
| 70. | 131812-67-4 | 1,3-Dioxolane, 2,4- | Okoumal ® | 0.00007600 |
| | | dimethyl-2-(5,6,7,8- | | |
| | | tetrahydro-5,5,8,8- | | |
| | | tetramethyl-2- | | |
| | | naphthalenyl)- | | |
| 71. | 106-02-5 | Oxacyyclohexadecan-2- | Exaltolide ® | 0.00006430 |
| | | one | | |
| 72. | 141773-73-1 | 1-Propanol, 2-[1-(3,3- | Helvetolide ® | 0.00005790 |
| | | dimethylcyclohexyl) | | |
| | | ethoxy]-2-methyl-, 1- | | |
| | | propanoate | | |
| 73. | 63314-79-4 | 5-Cyclopentadecen-1- | Delta | 0.00005650 |
| | | one, 3-methyl- | Muscenone | |
| 74. | 77-42-9 | 2-Penten-1-ol, 2-methyl- | cis-beta-Santalol | 0.00004810 |
| | | 5-[(1S,2R,4R)-2- | | |
| | | methyl-3-methylene- | | |
| | | bicyclo[2.2.1]hept-2-yl]-, | | |
| | | (2Z)- | | |
| 75. | 362467-67-2 | 2H-1,5-Benzodioxepin- | Azurone | 0.00004770 |
| | | 3(4H)-one, 7-(3-methyl- | | |
| | | butyl)- | | |
| 76. | 28371-99-5 | Ethanone, 1-(2,6,10- | Trimofix O | 0.00004580 |
| | | trimethyl-2,5,9- | | |
| | | cyclododecatrien-1-yl)- | | |
| 77. | 16223-63-5 | 1H-3a,6-Methanoazulene- | Khusimol | 0.00004400 |
| | | 3-methanol, octahydro- | | |
| | | 7,7-dimethyl-8-methylene-, | | |
| | | (3S,3aR,6R,8aS)- | | |
| 78. | 10461-98-0 | Benzeneacetonitrile, α- | Peonile | 0.00004290 |
| | | cyclohexylidene | | |
| 79. | 90-17-5 | Benzenemethanol, α- | Rosacetol | 0.00004240 |
| | | (trichloromethyl)-, 1- | | |
| | | acetate | | |
| 80. | 50607-64-2 | Benzoic acid, 2-[(2- | Mevantraal | 0.00004070 |
| | | methylpentylidene)amino]-, | | |
| | | methyl ester | | |
| 81. | 29895-73-6 | 5-Hydroxy-2-benzyl-1,3- | Acetal CD | 0.00004050 |
| | | dioxane | | |
| 82. | 94-47-3 | Benzoic acid, 2-phenylethyl | | |
| | | ester | | |
| 83. | 3100-36-5 | Cyclohexadec-8-en-1-one | Globanone ® | 0.00003310 |
| 84. | 37609-25-9 | 5-Cyclohexadecen-1-One | Ambretone | 0.00003310 |
| 85. | 66072-32-0 | Cyclohexanol, 4-(1,7,7- | Iso Bornyl | 0.00003010 |
| | | trimethylbicyclo[2.2.1] | | |
| | | hept-2-yl)- | | |
| 86. | 31906-04-4 | 3-Cyclohexene-1- | Lyral ® | 0.00002940 |
| | | carboxaldehyde, 4-(4- | | |
| | | hydroxy-4-methylpentyl)- | | |
| 87. | 21145-77-7 | Ethanone, 1-(5,6,7,8- | Musk Plus | 0.00002860 |
| | | tetrahydro-3,5,5,6,8,8- | | |
| | | hexamethyl-2- | | |
| | | naphthalenyl)- | | |
| 88. | 21145-77-7 | Ethanone, 1-(5,6,7,8- | Fixolide | 0.00002860 |
| | | tetrahydro-3,5,5,6,8,8- | | |
| | | hexamethyl-2- | | |
| | | naphthalenyl)- | | |
| 89. | 22442-01-9 | 2-Cyclopentadecan-1- | Muscenone | 0.00002770 |
| | | one, 3-methyl- | | |
| 90. | 109-29-5 | Oxacycloheptadecan-2- | Silvanone Ci | 0.00002600 |
| | | one | | |
| 91. | 101-94-0 | Benzeneacetic acid, 4- | Para Cresyl | 0.00002330 |
| | | methylphenyl ester | Phenyl Acetate | |
| 92. | 102-20-5 | Benzeneacetic acid, | Phenyl Ethyl | 0.00002300 |
| | | 2-phenylethyl ester | Phenyl Acetate | |
| 93. | 118562-73-5 | Cyclododecaneethanol, | Hydroxyambran | 0.00001800 |
| | | β-methyl- | | |
| 94. | 103-41-3 | 2-Propenoic acid, 3- | Benzyl Cinnamate | 0.00001050 |
| | | phenyl-, phenylmethyl | | |
| | | ester | | |
| 95. | 4707-47-5 | Benzoic acid, 2,4- | Veramoss/ | 0.00001050 |
| | | dihydroxy-3,6-dimethyl-, | LRG201/Evernyl | |
| | | methyl ester | | |
| 96. | 183551-83-9 | Naphtho[2,1-b]furan-6(7H)- | Myrrhone | 0.00000977 |
| | | one, 8,9-dihydro-1,5,8- | | |
| | | trimethyl-, (8R)- | | |
| 97. | 102-17-0 | Benzeneacetic acid, (4- | Para Anisyl | 0.00000813 |
| | | methoxyphenyl)methyl | Phenyl Acetate | |
| | | ester | | |
| 98. | 120-11-6 | Benzene, 2-methoxy-1- | Benzyl Iso | 0.00000676 |
| | | (phenylmethoxy)-4-(1- | | |
| | | propen-1-yl)- | | |
| 99. | 102-22-7 | Benzeneacetic acid, (2E)- | Geranyl | 0.00000645 |
| | | 3,7-dimethyl-2,6- | | |
| | | octadien-1-yl ester | | |
| 100. | 111879-80-2 | Oxacyclohexadec-12-en- | Habanolide 100% | 0.00000431 |
| | | 2-one, (12E)- | | |
| 101. | 87-22-9 | Benzoic acid, 2-hydroxy-, | Phenyl Ethyl | 0.00000299 |
| | | 2-phenylethyl ester | Salicylate | |
| 102. | 78-37-5 | 2-Propenoic acid, 3- | Linalyl Cinnamate | 0.00000174 |
| | | phenyl-, 1-ethenyl-1,5- | | |
| | | dimethyl-4-hexen-1-yl | | |
| | | ester | | |
| 103. | 28645-51-4 | Oxacycloheptadec-10-en- | Ambrettolide | 0.00000139 |
| | | 2-one | | |
| 104. | 123-69-3 | Oxacycloheptadec-8-en- | Ambrettolide | 0.00000136 |
| | | 2-one, (8Z)- | | |
| 105. | 3391-83-1 | 1,7- | Musk RI | 0.00000057 |
| | | Dioxacycloheptadecan-8- | | |
| | | one | | |
| 106. | 68527-79-7 | 7-Octen-2-ol, 8-(1H- | Indolene | 0.00000445 |
| | | indol-1-yl)-2,6-dimethyl- | | |
| 107. | 89-43-0 | Methyl 2-[(7-hydroxy- | Aurantinol | 0.00000100 |
| | | 3,7-dimethyloctylidene) | | |
| | | amino]benzoate | | |
| 108. | 54982-83-1 | 1,4- | Zenolide | 0.00000000834 |
| | | Dioxacyclohexadecane- | | |
| | | 5,16-dione | | |
| 109. | 105-95-3 | 1,4- | Ethylene | 0.00000000313 |
| | | Dioxacycloheptadecane- | Brassylate | |
| | | 5,17-dione | | |
| 110. | 3681-73-0 | Hexadecanoic acid, (2E)- | Hexarose | 0.00000000300 |
| | | 3,7-dimethyl-2,6-octadien- | | |
| | | 1-yl ester | | |
| 111. | 4159-29-9 | Phenol, 4-[3-(benzoyloxy)- | Coniferyl benzoate | 0.00000000170 |
| | | 1-propen-1-yl]-2-methoxy- | | |
| 112. | 144761-91-1 | Benzoic acid, 2-[(1-hydroxy- | Trifone DIPG | 0.00000000093 |
| | | 3-phenylbutyl)amino]-, | | |
| | | methyl ester | | |

¹ Non-limiting examples of alternative qualities from various suppliers can be purchased under the following tradenames: Kharismal ® Super (IFF), Kharismal ® (IFF), Hedione ® (Firmenich), Hedione ® HC (Firmenich), Paradisone (Firmenich), Cepionate (Zenon), Super cepionate (Zenon), Claigeon ® (Zenon).
*Vapor Pressures are acquired as described in the Test Methods Section.
**Origin: The low volatile fragrance materials may be obtained from one or more of the following companies: Firmenich (Gebeva, Switzerland), Symrise AG (Holzminden, Germany), Givaudan (Argenteuil, France), IFF (Hazlet, New Jersey), Bedoukian (Danbury, Coonneticut), Sigma Aldrich (St. Louis, Missouri), Millennium Specialty Chemicals (Olympia Fields, Illinois), Polarone International (Jersey City, New Jersey), and Aroma & Flavor Specialties (Danbury, Conneticut).

TABLE 1B
Low Volatile Natural Oils.

| No. | Natural oil | Supplier |
|---|---|---|
| 1. | Beeswax Absolute | Robertet |
| 2. | Cedarwood Sawdust SFE | Firmenich |
| 3. | Cedarwood Oil Rect | Firmenich |
| 4. | Cedarwood Texas Light | H. Reynaud & Fils |
| 5. | Ciste Absolute | IFF |
| 6. | Cocoa Colorless Oil | Robertet |
| 7. | Cypriol Coeur Essence | Robertet |
| 8.. | Guaiacwood Oil | Global Essence Inc |
| 9. | Incense Wood Natural | Robertet |
| 10. | Orris CO2 Extract | Mane |
| 11. | Patchouli Oil | IFF |
| 12. | Tolu Baume Res | Robertet |
| 13. | Vanilla Absolute | Robertet |
| 14. | Vanilla CO2 Absolute | Robertet |
| 15. | Vetivert Oil | IFF |
| 16. | Vetyvert Acetate | Robertet |

### Suppliers:

Firmenich, Geneva, Switzerland
Global Essence Inc, New Jersey, USA
H. Reynaud & Fils, Montbrun-les-Bains, France
IFF, Hazlet, New Jersey, USA
Mane, Le Bar-sur-Loup, France
Robertet, Grasse, France

Exemplary low volatile fragrance materials selected from the group of Tables 1A or 1B Low Volatile Fragrance Materials are preferred. However, it is understood by one skilled in the art that other low volatile fragrance materials, not recited in Tables 1A or 1B, would also fall within the scope of the present invention, so long as they have a vapor pressure less than 0.001 Torr (0.000133 kPa) at 25° C.

### (ii) Moderate Volatile Fragrance Materials

The fragrance component includes at least one moderate volatile fragrance material or aggregate of volatile fragrance materials having a vapor pressure in the range of 0.1 Torr to 0.001 Torr (0.0133 kPa to 0.000133 kPa) at 25° C. In some examples, the composition according to the present disclosure can include at least 3 moderate volatile fragrance materials, or at least 5 moderate volatile fragrance materials, or at least 7 moderate volatile fragrance materials. If there are more than one moderate volatile fragrance materials, then the ranges provided hereinabove cover the total of all of the moderate volatile fragrance materials. Suitable examples of moderate volatile fragrances materials are provided in Table 2A and 2B below.

Preferable examples of moderate volatile fragrance materials having a vapor pressure in the range of 0.1 Torr to 0.001 Torr (0.0133 kPa to 0.000133 kPa) at 25° C. are provided in Table 2A and 2B. Preferably, the moderate volatile fragrance material is selected from at least 1 material, or at least 2 materials, or at least 3 materials, or at least 5 materials, or at least 7 moderate volatile fragrance materials as disclosed in Table 2A. Natural fragrance materials or oils having an aggregate vapour pressure between 0.1 Torr to 0.001 Torr (0.0133 kPa to 0.000133 kPa) at 25° C. are provided in Table 2B. Moderate Volatile Natural Oils.

TABLE 2A
Moderate Volatile Fragrance Materials

| | | | | Vapor |
|---|---|---|---|---|
| | | | | Pressure |
| | CAS | | Common | (Torr at |
| No. | Number | IUPAC Name | Name** | 25° C.)* |
| 1. | 24168-70-5 | Pyrazine, 2-methoxy- | Methoxyiso- | 0.09950000 |
| | | 3-(1-methylpropyl)- | butylpyrazine | |
| 2. | 89-79-2 | Cyclohexanol, 5- | Iso-Pulegol | 0.09930000 |
| | | methyl-2-(1- | | |
| | | methylethenyl)-, | | |
| | | (1R,2S,5R)- | | |
| 3. | 112-12-9 | 2-Undecanone | Methyl Nonyl | 0.09780000 |
| | | | Ketone | |
| 4. | 103-05-9 | Benzenepropanol, | Phenyl Ethyl | 0.09770000 |
| | | α,α-dimethyl- | Dimethyl | |
| | | | Carbinol | |
| 5. | 125-12-2 | Bicyclo[2.2.1]heptan- | Iso Bornyl | 0.09590000 |
| | | 2-ol, 1,7,7- | Acetate | |
| | | trimethyl-, 2-acetate, | | |
| | | (1R,2R,4R)-rel- | | |
| 6. | 78-70-6 | 1,6-Octadien-3-ol, | Linalool | 0.09050000 |
| | | 3,7-dimethyl- | | |
| 7. | 101-97-3 | Benzeneacetic acid, | Ethyl Phenyl | 0.08970000 |
| | | ethyl ester | Acetate | |
| 8. | 100-86-7 | Benzeneethanol, | Dimethyl | 0.08880000 |
| | | α,α-dimethyl- | Benzyl | |
| | | | Carbinol | |
| 9. | 188570-78-7 | Cyclopropane | Montaverdi | 0.08640000 |
| | | carboxylic acid, (3Z)- | | |
| | | 3-hexen-1-yl ester | | |
| 10. | 67634-25-7 | 3-Cyclohexene-1- | Floralate | 0.08500000 |
| | | methanol, 3,5- | | |
| | | dimethyl-, 1-acetate | | |
| 11. | 112-44-7 | Undecanal | Undecyl | 0.08320000 |
| | | | Aldehyde | |
| 12. | 32669-00-4 | Ethanone, 1-(3- | Tanaisone ® | 0.08150000 |
| | | cycloocten-1-yl)- | | |
| 13. | 98-53-3 | Cyclohexanone, 4- | Patchi | 0.07780000 |
| | | (1,1-dimethylethyl)- | | |
| 14. | 35854-86-5 | 6-Nonen-1-ol, (6Z)- | cis-6-None-1- | 0.07770000 |
| | | | ol | |
| 15. | 5331-14-6 | Benzene, (2- | Butyl | 0.07760000 |
| | | butoxyethyl)- | phenethyl ether | |
| 16. | 80-57-9 | Bicyclo[3.1.1]hept- | Verbenone | 0.07730000 |
| | | 3-en-2-one, 4,6,6- | | |
| | | trimethyl- | | |
| 17. | 22471-55-2 | Cyclohexanecarboxylic | Thesaron | 0.07670000 |
| | | acid, 2,2,6-trimethyl-, | | |
| | | ethyl ester, (1R,6S)- | | |
| | | rel- | | |
| 18. | 60-12-8 | Benzeneethanol | Phenethyl | 0.07410000 |
| | | | alcohol | |
| 19. | 106-26-3 | 2,6-Octadienal, 3,7- | Neral | 0.07120000 |
| | | dimethyl-, (2Z)- | | |
| 20. | 5392-40-5 | 2,6-Octadienal, 3,7- | Citral | 0.07120000 |
| | | dimethyl- | | |
| 21. | 89-48-5 | Cyclohexanol, 5- | Menthyl | 0.07070000 |
| | | methyl-2-(1- | Acetate | |
| | | methylethyl)-, 1- | | |
| | | acetate, (1R,2S,5R)- | | |
| | | rel- | | |
| 22. | 119-36-8 | Benzoic acid, 2- | Methyl | 0.07000000 |
| | | hydroxy-, methyl ester | salicylate | |
| 23. | 104-46-1 | Benzene, 1-methoxy- | Anethol | 0.06870000 |
| | | 4-(1E)-1-propen-1-yl- | | |
| 24. | 7549-37-3 | 2,6-Octadiene, 1,1- | Citral Dimethyl | 0.06780000 |
| | | dimethoxy-3,7- | Acetal | |
| | | dimethyl- | | |
| 25. | 25225-08-5 | Cyclohexanemethanol, | Aphermate | 0.06780000 |
| | | α,3,3-trimethyl-, | | |
| | | 1-formate | | |
| 26. | 3913-81-3 | 2-Decenal, (2E)- | 2-Decene-1-al | 0.06740000 |
| 27. | 15373-31-6 | 3-Cyclopentene-1- | Cantryl ® | 0.06700000 |
| | | acetonitrile, 2,2,3- | | |
| | | trimethyl- | | |
| 28 | 6485-40-1 | 2-Cyclohexen-1-one, | Laevo carvone | 0.06560000 |
| | | 2-methyl-5-(1- | | |
| | | methylethenyl)-, | | |
| | | (5R)- | | |
| 29 | 16587-71-6 | Cyclohexanone, 4- | Orivone | 0.06490000 |
| | | (1,1-dimethylpropyl)- | | |
| 30. | 62406-73-9 | 6,10- | Opalal CI | 0.06290000 |
| | | Dioxaspiro[4.5] | | |
| | | decane,8,8-dimethyl- | | |
| | | 7-(1-methylethyl)- | | |
| 31 | 3720-16-9 | 2-Cyclohexen-1-one, | Livescone | 0.06270000 |
| | | 3-methyl-5-propyl- | | |
| 32. | 13816-33-6 | Benzonitrile, 4-(1- | Cumin Nitrile | 0.06230000 |
| | | methylethyl)- | | |
| 33. | 67019-89-0 | 2,6-Nonadienenitrile | Violet Nitrile | 0.06200000 |
| 34. | 53398-85-9 | Butanoic acid, 2- | cis-3-Hexenyl | 0.06130000 |
| | | methyl-, (3Z)-3- | Alpha Methyl | |
| | | hexen-1-yl ester | Butyrate | |
| 35. | 208041-98-9 | n/a | Jasmonitrile | 0.05920000 |
| 36. | 16510-27-3 | Benzene, 1- | Toscanol | 0.05870000 |
| | | (cyclopropylmethyl)- | | |
| | | 4-methoxy- | | |
| 37 | 111-80-8 | 2-Nonynoic acid, | Methyl Octine | 0.05680000 |
| | | methyl ester | Carbonate | |
| 38. | 103-45-7 | Acetic acid, 2- | Phenyl Ethyl | 0.05640000 |
| | | phenylethyl ester | Acetate | |
| 39. | 2550-26-7 | 2-Butanone, 4-phenyl- | Benzyl Acetone | 0.05570000 |
| 40 | 13491-79-7 | Cyclohexanol, 2- | Verdol | 0.05430000 |
| | | (1,1-dimethylethyl)- | | |
| 41. | 7786-44-9 | 2,6-Nonadien-1-ol | 2,6-Nonadien- | 0.05370000 |
| | | | 1-ol | |
| 42. | 103-28-6 | Propanoic acid, 2- | Benzyl Iso | 0.05130000 |
| | | methyl-, | Butyrate | |
| | | phenylmethyl ester | | |
| 43. | 104-62-1 | Formic acid, 2- | Phenyl Ethyl | 0.05050000 |
| | | phenylethyl ester | Formate | |
| 44. | 28462-85-3 | Bicyclo[2.2.1]heptan- | Humus Ether | 0.04870000 |
| | | 2-ol, 1,2,3,3- | | |
| | | tetramethyl-, | | |
| | | (1R,2R,4S)-rel- | | |
| 45. | 122-03-2 | Benzaldehyde, 4-(1- | Cuminic | 0.04820000 |
| | | methylethyl)- | Aldehyde | |
| 46. | 358331-95-0 | 2,5-Octadien-4-one, | Pomarose | 0.04810000 |
| | | 5,6,7-trimethyl-, | | |
| | | (2E)- | | |
| 47. | 562-74-3 | 3-Cyclohexen-1-ol, | Terpinenol-4 | 0.04780000 |
| | | 4-methyl-1-(1- | | |
| | | methylethyl)- | | |
| 48. | 68527-77-5 | 3-Cyclohexene-1- | Isocyclo- | 0.04640000 |
| | | methanol, 2,4,6- | geraniol | |
| | | trimethyl- | | |
| 49. | 35852-46-1 | Pentanoic acid, (3Z)- | Cis-3-Hexenyl | 0.04580000 |
| | | 3-hexen-1-yl ester | Valerate | |
| 50 | 2756-56-1 | Bicyclo[2.2.1]heptan- | Iso Bornyl | 0.04540000 |
| | | 2-ol, 1,7,7-trimethyl-, | Propionate | |
| | | 2-propanoate, | | |
| | | (1R,2R,4R)-rel- | | |
| 51. | 14374-92-6 | Benzene, 1-methyl- | Verdoracine | 0.04460000 |
| | | 4-(1-methylethyl)-2- | | |
| | | (1-propen-1-yl)- | | |
| 52. | 6784-13-0 | 3-Cyclohexene-1- | Limonenal | 0.04380000 |
| | | propanal, β,4- | | |
| | | dimethyl- | | |
| 53. | 8000-41-7 | 2-(4-methyl-1- | Alpha | 0.04320000 |
| | | cyclohex-3-enyl) | Terpineol | |
| | | propan-2-ol | | |
| 54. | 41884-28-0 | 1-Hexanol, 5-methyl- | Tetrahydro | 0.04230000 |
| | | 2-(1-methylethyl)-, | Lavandulol | |
| | | (2R)- | | |
| 55. | 22457-23-4 | 3-Heptanone, 5- | Stemone ® | 0.04140000 |
| | | methyl-, oxime | | |
| 56 | 104-50-7 | 2(3H)-Furanone, 5- | Gamma | 0.04080000 |
| | | butyldihydro- | Octalactone | |
| 57. | 143-08-8 | 1-Nonanol | Nonyl Alcohol | 0.04070000 |
| 58. | 3613-30-7 | Octanal, 7-methoxy- | Methoxy- | 0.04020000 |
| | | 3,7-dimethyl- | citronellal | |
| 59 | 67634-00-8 | Acetic acid, 2-(3- | Allyl Amyl | 0.04000000 |
| | | methylbutoxy)-, 2- | Glycolate | |
| | | propen-1-yl ester | | |
| 60 | 464-45-9 | Bicyclo[2.2.1]heptan- | 1-Borneol | 0.03980000 |
| | | 2-ol, 1,7,7-trimethyl-, | | |
| | | (1S,2R,4S)- | | |
| 61. | 124-76-5 | Bicyclo[2.2.1 ]heptan- | 1.7.7- | 0.03980000 |
| | | 2-ol, 1,7,7- | Trimethyl- | |
| | | trimethyl-, | Bicyclo-1.2.2- | |
| | | (1R,2R,4R)-rel- | Heptanol-2 | |
| 62. | 67874-72-0 | Cyclohexanol, 2-(1,1- | Coniferan | 0.03980000 |
| | | dimethylpropyl)-, 1- | | |
| | | acetate | | |
| 63. | 80-26-2 | 3-Cyclohexene-1- | Terpinyl | 0.03920000 |
| | | methanol, α,α,4- | Acetate | |
| | | trimethyl-, 1-acetate | | |
| 64. | 498-81-7 | Cyclohexanemethanol, | Dihydro | 0.03920000 |
| | | α,α,4-trimethyl- | Terpineol | |
| 65 | 112-45-8 | 10-Undecenal | Undecylenic | 0.03900000 |
| | | | aldehyde | |
| 66. | 35044-57-6 | 2,4-Cyclohexadiene- | Ethyl | 0.03880000 |
| | | 1-carboxylic acid, | Safranate | |
| | | 2,6,6-trimethyl-, | | |
| | | ethyl ester | | |
| 67 | 106-21-8 | 1-Octanol, 3,7- | Dimethyl | 0.03860000 |
| | | dimethyl- | Octanol | |
| 68. | 84560-00-9 | Cyclopentanol, 2- | Cyclopentol | 0.03790000 |
| | | pentyl- | | |
| 69. | 82461-14-1 | Furan, tetrahydro- | Rhubafuran | 0.03780000 |
| | | 2,4-dimethyl-4- | | |
| | | phenyl- | | |
| 70. | 56011-02-0 | Benzene, [2-(3- | Phenyl Ethyl | 0.03690000 |
| | | methylbutoxy)ethyl]- | Isoamyl Ether | |
| 71. | 103-37-7 | Butanoic acid, | Benzyl | 0.03660000 |
| | | phenylmethyl ester | Butyrate | |
| 72. | 6378-65-0 | Hexyl hexanoate | Hexyl | 0.03490000 |
| | | | hexanoate | |
| 73. | 118-61-6 | Benzoic acid, 2- | Ethyl | 0.03480000 |
| | | hydroxy-, ethyl ester | salicylate | |
| 74. | 98-52-2 | Cyclohexanol, 4- | Patchon | 0.03480000 |
| | | (1,1-dimethylethyl)- | | |
| 75. | 115-99-1 | 1,6-Octadien-3-ol, | Linalyl | 0.03440000 |
| | | 3,7-dimethyl-, 3- | Formate | |
| | | formate | | |
| 76. | 112-54-9 | Dodecanal | Lauric | 0.03440000 |
| | | | Aldehyde | |
| 77. | 53046-97-2 | 3,6-Nonadien-1-ol, | 3,6 Nonadien- | 0.03360000 |
| | | (3Z,6Z)- | 1-ol | |
| 78. | 76649-25-7 | 3,6-Nonadien-1-ol | 3,6-Nonadien- | 0.03360000 |
| | | | 1-ol | |
| 79. | 141-25-3 | 3,7-Dimethyloct-6- | Rhodinol | 0.03290000 |
| | | en-1-ol | | |
| 80. | 1975-78-6 | Decanenitrile | Decanonitrile | 0.03250000 |
| 81. | 2216-51-5 | Cyclohexanol, 5- | L-Menthol | 0.03230000 |
| | | methyl-2-(1- | | |
| | | methylethyl)-, | | |
| | | (1R,2S,5R)- | | |
| 82. | 3658-77-3 | 4-hydroxy-2,5- | Pineapple | 0.03200000 |
| | | dimethylfuran-3-one | Ketone | |
| 83. | 103-93-5 | Propanoic acid, 2- | Para Cresyl | 0.03120000 |
| | | methyl-, 4- | iso-Butyrate | |
| | | methylphenyl ester | | |
| 84. | 24717-86-0 | Propanoic acid, | Abierate | 0.03110000 |
| | | 2-methyl-, | | |
| | | (1R,2S,4R)-1,7,7- | | |
| | | trimethylbicyclo | | |
| | | [2.2.1]hept-2-yl | | |
| | | ester, rel- | | |
| 85. | 67845-46-9 | Acetaldehyde, 2-(4- | Aldehyde XI | 0.03090000 |
| | | methylphenoxy)- | | |
| 86 | 67883-79-8 | 2-Butenoic acid, 2- | Cis-3-Hexenyl | 0.03060000 |
| | | methyl-, (3Z)-3- | Tiglate | |
| | | hexen-1-yl ester, | | |
| | | (2E)- | | |
| 87 | 33885-51-7 | Bicyclo[3.1.1]hept- | Pino | 0.03040000 |
| | | 2-ene-2-propanal, | Acetaldehyde | |
| | | 6,6-dimethyl- | | |
| 88 | 105-85-1 | 6-Octen-1-ol, 3,7- | Citronellyl | 0.03000000 |
| | | dimethyl-, 1-formate | Formate | |
| 89. | 70214-77-6 | 2-Nonanol, 6,8- | Nonadyl | 0.03010000 |
| | | dimethyl- | | |
| 90 | 215231-33-7 | Cyclohexanol, 1- | Rossitol | 0.02990000 |
| | | methyl-3-(2- | | |
| | | methylpropyl)- | | |
| 91 | 120-72-9 | 1H-Indole | Indole | 0.02980000 |
| 92. | 2463-77-6 | 2-Undecenal | 2-Undecene- | 0.02970000 |
| | | | 1-al | |
| 93. | 675-09-2 | 2H-Pyran-2-one, | Levistamel | 0.02940000 |
| | | 4,6-dimethyl- | | |
| 94. | 98-55-5 | 3-Cyclohexene-1- | Alpha- | 0.02830000 |
| | | methanol, α,α,4- | Terpineol | |
| | | trimethyl- | | |
| 95 | 81786-73-4 | 3-Hepten-2-one, | Koavone | 0.02750000 |
| | | 3,4,5,6,6- | | |
| | | pentamethyl-, (3Z)- | | |
| 96. | 122-97-4 | Benzenepropanol | Phenyl Propyl | 0.02710000 |
| | | | Alcohol | |
| 97. | 39212-23-2 | 2(3H)-Furanone, 5- | Methyl | 0.02700000 |
| | | butyldihydro-4- | Octalactone | |
| | | methyl- | | |
| 98 | 53767-93-4 | 7-Octen-2-ol, 2,6- | Dihydro | 0.02690000 |
| | | dimethyl-, 2-acetate | Terpinyl | |
| | | | Acetate | |
| 99. | 35044-59-8 | 1,3-Cyclohexadiene- | Ethyl | 0.02660000 |
| | | 1-carboxylic acid, | Safranate | |
| | | 2,6,6-trimethyl-, | | |
| | | ethyl ester | | |
| 100. | 104-55-2 | 2-Propenal, 3-phenyl- | Cinnamic | 0.02650000 |
| | | | Aldehyde | |
| 101. | 144-39-8 | 1,6-Octadien-3-ol, | Linalyl | 0.02630000 |
| | | 3,7-dimethyl-, 3- | Propionate | |
| | | propanoate | | |
| 102. | 61931-80-4 | 1,6-Nonadien-3-ol, 3, | 3,7-Dimethyl- | 0.02630000 |
| | | 7-dimethyl-, 3-acetate | 1,6-nonadien- | |
| | | | 3-yl acetate | |
| 103. | 102-13-6 | Benzeneacetic acid, | Iso Butyl | 0.02630000 |
| | | 2-methylpropyl ester | Phenylacetate | |
| 104. | 65443-14-3 | Cyclopentanone, 2,2, | Veloutone | 0.02610000 |
| | | 5-trimethyl-5-pentyl- | | |
| 105. | 141-12-8 | 2,6-Octadien-1-ol, | Neryl Acetate | 0.02560000 |
| | | 3,7-dimethyl-, 1- | | |
| | | acetate, (2Z)- | | |
| 106. | 105-87-3 | 2,6-Octadien-1-ol, | Geranyl | 0.02560000 |
| | | 3,7-dimethyl-, 1- | acetate | |
| | | acetate, (2E)- | | |
| 107. | 68141-17-3 | Undecane, 1,1- | Methyl Nonyl | 0.02550000 |
| | | dimethoxy-2-methyl- | Acetaldehyde | |
| | | | Dimethyl | |
| | | | Acetal | |
| 108. | 2206-94-2 | Benzenemethanol, | Indocolore | 0.02550000 |
| | | α-methylene-, | | |
| | | 1-acetate | | |
| 109. | 10528-67-3 | Cyclohexanepropanol, | Cyclohexyl- | 0.02550000 |
| | | α-methyl- | magnol | |
| 110. | 123-11-5 | Benzaldehyde, 4- | Anisic | 0.02490000 |
| | | methoxy- | Aldehyde | |
| 111. | 57576-09-7 | Cyclohexanol, 5- | Iso Pulegol | 0.02480000 |
| | | methyl-2-(1- | Acetate | |
| | | methylethenyl)-, 1- | | |
| | | acetate, (1R,2S,5R)- | | |
| 112. | 51566-62-2 | 6-Octenenitrile, 3,7- | Citronellyl | 0.02470000 |
| | | dimethyl- | Nitrile | |
| 113. | 60335-71-9 | 2H-Pyran, 3,6- | Rosyrane | 0.02470000 |
| | | dihydro-4-methyl- | Super | |
| | | 2-phenyl- | | |
| 114. | 30385-25-2 | 6-Octen-2-ol, 2,6- | Dihydro- | 0.02440000 |
| | | dimethyl- | myrcenol | |
| 115. | 101-84-8 | Benzene, 1,1′-oxybis- | Diphenyl | 0.02230000 |
| | | | Oxide | |
| 116. | 136-60-7 | Benzoic acid, butyl | Butyl | 0.02170000 |
| | | ester | Benzoate | |
| 117. | 93939-86-7 | 5,8-Methano-2H-1- | Rhuboflor | 0.02120000 |
| | | benzopyran, 6- | | |
| | | ethylideneoctahydro- | | |
| 118. | 83926-73-2 | Cyclohexanepropanol, | Coranol | 0.02100000 |
| | | α,α-dimethyl- | | |
| 119. | 125109-85-5 | Benzenepropanal, β- | Florhydral | 0.02070000 |
| | | methyl-3-(1- | | |
| | | methylethyl)- | | |
| 120. | 104-21-2 | Benzenemethanol, | Anisyl | 0.02050000 |
| | | 4-methoxy-, 1-acetate | Acetate | |
| 121. | 1365-19-1 | 2-Furanmethanol, 5- | Linalool | 0.02050000 |
| | | ethenyltetrahydro- | Oxide | |
| | | α,α,5-trimethyl- | | |
| 122. | 137-03-1 | Cyclopentanone, 2- | Frutalone | 0.02040000 |
| | | heptyl- | | |
| 123. | 2563-07-7 | Phenol, 2-ethoxy-4- | Ultravanil | 0.02030000 |
| | | methyl- | | |
| 124. | 1128-08-1 | 2-Cyclopenten-1-one, | Dihydro- | 0.02020000 |
| | | 3-methyl-2-pentyl- | jasmone | |
| 125. | 7493-57-4 | Benzene, [2-(1- | Acetaldehyde | 0.01990000 |
| | | propoxyethoxy)ethyl]- | | |
| 126. | 141-25-3 | 7-Octen-1-ol, 3,7- | Rhodinol | 0.01970000 |
| | | dimethyl- | | |
| 127. | 216970-21-7 | Bicyclo[4.3.1]decane, | 3-Methoxy-7, | 0.01960000 |
| | | 3-methoxy-7,7- | 7-dimethyl- | |
| | | dimethyl-10- | 10-methylene- | |
| | | methylene- | bicyclo[4.3.1] | |
| | | | decane | |
| 128. | 319002-92-1 | Propanoic acid, 2-(1, | Sclareolate ® | 0.01960000 |
| | | 1-dimethylpropoxy)-, | | |
| | | propyl ester, (2S)- | | |
| 129. | 85-91-6 | Benzoic acid, 2- | Dimethyl | 0.01930000 |
| | | (methylamino)-, | anthranilate | |
| | | methyl | | |
| 130. | 13828-37-0 | Cyclohexanemethanol, | Mayol | 0.01920000 |
| | | 4-(1-methylethyl)-, | | |
| | | cis- | | |
| 131. | 26330-65-4 | (E)-6-ethyl-3- | Super Muguet | 0.01850000 |
| | | methyloct-6-en-1-ol | | |
| 132. | 7540-51-4 | 6-Octen-1-ol, 3,7- | L-Citronellol | 0.01830000 |
| | | dimethyl-, (3S)- | | |
| 133. | 106-22-9 | 6-Octen-1-ol, 3,7- | Citronellol | 0.01830000 |
| | | dimethyl- | | |
| 134. | 543-39-5 | 7-Octen-2-ol, 2- | Myrcenol | 0.01820000 |
| | | methyl-6-methylene- | | |
| 135. | 7775-00-0 | Benzenepropanal, 4- | Cyclemax | 0.01820000 |
| | | (1-methylethyl)- | | |
| 136. | 18479-54-4 | 4,6-Octadien-3-ol, | Muguol | 0.01800000 |
| | | 3,7-dimethyl- | | |
| 137. | 29214-60-6 | Octanoic acid, 2- | Gelsone | 0.01790000 |
| | | acetyl-, ethyl ester | | |
| 138. | 1209-61-6 | 5-Oxatricyclo | Tobacarol | 0.01730000 |
| | | [8.2.0.04,6]dodecane, | | |
| | | 4,9,12,12- | | |
| | | tetramethyl- | | |
| 139. | 57934-97-1 | 2-Cyclohexene-1- | Givescone | 0.01710000 |
| | | carboxylic acid, 2- | | |
| | | ethyl-6,6-dimethyl-, | | |
| | | ethyl ester | | |
| 140. | 14901-07-6 | 3-Buten-2-one, 4- | Beta-Ionone | 0.01690000 |
| | | (2,6,6-trimethyl-1- | | |
| | | cyclohexen-1-yl)-, | | |
| | | (3E)- | | |
| 141. | 64001-15-6 | 4,7-Methano-1H- | Dihydro | 0.01630000 |
| | | inden-5-ol, | Cyclacet | |
| | | octahydro-, 5-acetate | | |
| 142. | 95-41-0 | 2-Cyclopenten-1-one, | Iso Jasmone | 0.01600000 |
| | | 2-hexyl- | T | |
| 143. | 134-20-3 | Benzoic acid, 2- | Methyl | 0.01580000 |
| | | amino-, methyl ester | Anthranilate | |
| 144. | 100-06-1 | Ethanone, 1-(4- | Para Methoxy | 0.01550000 |
| | | methoxyphenyl)- | Acetophenone | |
| 145. | 105-86-2 | 2,6-Octadien-1-ol, | Geranyl | 0.01540000 |
| | | 3,7-dimethyl-, | Formate | |
| | | 1-formate, (2E)- | | |
| 146. | 154171-77-4 | Spiro[1,3-dioxolane- | Ysamber K ® | 0.01470000 |
| | | 2,8′(5′H)-[2H-2,4a] | | |
| | | methanonaphthalene], | | |
| | | hexahydro-1′,1′,5′,5′- | | |
| | | tetramethyl-, | | |
| | | (2′S,4′aS,8′aS)-(9CI) | | |
| 147. | 154171-76-3 | Spiro[1,3-dioxolane- | Ysamber | 0.01470000 |
| | | 2,8′(5′H)-[2H-2,4a] | | |
| | | methanonaphthalene], | | |
| 148. | 127-41-3 | 3-Buten-2-one, 4-(2, | Alpha-Ionone | 0.01440000 |
| | | 6,6-trimethyl-2- | | |
| | | cyclohexen-1-yl)-, | | |
| | | (3E)- | | |
| 149. | 151-05-3 | Benzeneethanol, | Dimethyl | 0.01390000 |
| | | α,α-dimethyl-, 1- | Benzyl | |
| | | acetate | Carbinyl | |
| | | | Acetate | |
| 150. | 2500-83-6 | 4,7-Methano-1H- | Flor Acetate | 0.01370000 |
| | | inden-5-ol, | | |
| | | 3a,4,5,6,7,7a- | | |
| | | hexahydro-, | | |
| | | 5-acetate | | |
| 151. | 150-84-5 | 6-Octen-1-ol, 3,7- | Citronellyl | 0.01370000 |
| | | dimethyl-, 1-acetate | acetate | |
| 152. | 30310-41-9 | 2H-Pyran, tetrahydro- | Pelargene | 0.01350000 |
| | | 2-methyl-4- | | |
| | | methylene-6-phenyl- | | |
| 153. | 68845-00-1 | Bicyclo[3.3.1]nonane, | Boisiris | 0.01350000 |
| | | 2-ethoxy-2,6,6- | | |
| | | trimethyl-9- | | |
| | | methylene- | | |
| 154. | 106-24-1 | 2,6-Octadien-1-ol, | Geraniol | 0.01330000 |
| | | 3,7-dimethyl-, (2E)- | | |
| 155. | 106-25-2 | 2,6-Octadien-1-ol, | Nerol | 0.01330000 |
| | | 3,7-dimethyl-, (2Z)- | | |
| 156. | 75975-83-6 | Bicyclo[7.2.0]undec- | Vetyvenal | 0.01280000 |
| | | 4-ene, 4,11,11- | | |
| | | trimethyl-8- | | |
| | | methylene-, | | |
| | | (1R,4E,9S)- | | |
| 157. | 19870-74-7 | 1H-3a,7- | Cedryl methyl | 0.01280000 |
| | | Methanoazulene, | ether | |
| | | octahydro-6-methoxy- | | |
| | | 3,6,8,8-tetramethyl-, | | |
| | | (3R,3aS,6S,7R,8aS)- | | |
| 158. | 87-44-5 | Bicyclo[7.2.0]undec- | Caryophyllene | 0.01280000 |
| | | 4-ene, 4,11,11- | Extra | |
| | | trimethyl-8- | | |
| | | methylene-, | | |
| | | (1R,4E,9S)- | | |
| 159. | 54440-17-4 | 1H-Inden-1-one, | Safraleine | 0.01260000 |
| | | 2,3-dihydro-2,3,3- | | |
| | | trimethyl- | | |
| 160. | 110-98-5 | 2-Propanol, 1,1′- | Dipropylene | 0.01250000 |
| | | oxybis- | Glycol | |
| 161. | 41890-92-0 | 2-Octanol, 7-methoxy- | Osyrol® | 0.01250000 |
| | | 3,7-dimethyl- | | |
| 162. | 71077-31-1 | 4,9-Decadienal, 4,8- | Floral Super | 0.01230000 |
| | | dimethyl- | | |
| 163. | 65-85-0 | Benzoic Acid | Benzoic Acid | 0.01220000 |
| 164. | 61444-38-0 | 3-Hexenoic acid, | cis-3-hexenyl- | 0.01220000 |
| | | (3Z)-3-hexen-1-yl | cis-3- | |
| | | ester, (3Z)- | hexenoate | |
| 165. | 116044-44-1 | Bicyclo[2.2.1]hept- | Herbanate | 0.01210000 |
| | | 5-ene-2-carboxylic | | |
| | | acid, 3-(1- | | |
| 166. | 104-54-1 | 2-Propen-1-ol, 3- | Cinnamic | 0.01170000 |
| | | phenyl- | Alcohol | |
| 167. | 78-35-3 | Propanoic acid, 2- | Linalyl | 0.01170000 |
| | | methyl-, 1-ethenyl- | Isobutyrate | |
| | | 1,5-dimethyl-4- | | |
| | | hexen-1-yl ester | | |
| 168. | 23495-12-7 | Ethanol, 2-phenoxy-, | Phenoxy Ethyl | 0.01130000 |
| | | 1-propanoate | Propionate | |
| 169. | 103-26-4 | 2-Propenoic acid, 3- | Methyl | 0.01120000 |
| | | phenyl-, methyl ester | Cinnamate | |
| 170. | 67634-14-4 | Benzenepropanal, 2- | Florazon | 0.01110000 |
| | | ethyl-α,α-dimethyl- | (ortho-isomer) | |
| 171. | 5454-19-3 | Propanoic acid, | N-Decyl | 0.01100000 |
| | | decyl ester | Propionate | |
| 172. | 93-16-3 | Benzene, 1,2- | Methyl Iso | 0.01100000 |
| | | dimethoxy-4-(1- | Eugenol | |
| | | propen-1-yl)- | | |
| 173. | 81782-77-6 | 3-Decen-5-ol, 4- | 4-Methyl-3- | 0.01070000 |
| | | methyl- | decen-5-ol | |
| 174 | 67845-30-1 | Bicyclo[2.2.2]oct-5- | Maceal | 0.01060000 |
| | | ene-2-carboxaldehyde, | | |
| | | 6-methyl-8-(1- | | |
| | | methylethyl)- | | |
| 175. | 97-53-0 | Phenol, 2-methoxy- | Eugenol | 0.01040000 |
| | | 4-(2-propen-1-yl)- | | |
| 176. | 120-57-0 | 1,3-Benzodioxole-5- | Heliotropin | 0.01040000 |
| | | carboxaldehyde | | |
| 177. | 93-04-9 | Naphthalene, 2- | Beta Naphthyl | 0.01040000 |
| | | methoxy- | Methyl Ether | |
| | | | Extra 99 | |
| 178. | 4826-62-4 | 2-Dodecenal | 2 Dodecene- | 0.01020000 |
| | | | 1-al | |
| 179. | 20407-84-5 | 2-Dodecenal, (2E)- | Aldehyde | 0.01020000 |
| | | | Mandarin | |
| 180. | 5462-06-6 | Benzenepropanal, 4- | Canthoxal | 0.01020000 |
| | | methoxy-α-methyl- | | |
| 181. | 94-60-0 | 1,4-Cyclohexane- | Dimethyl 1,4- | 0.01020000 |
| | | dicarboxylic acid, | cyclohexane- | |
| | | 1,4-dimethyl ester | dicarboxylate | |
| 182. | 57378-68-4 | 2-Buten-1-one, 1- | delta- | 0.01020000 |
| | | (2,6,6-trimethyl-3- | Damascone | |
| | | cyclohexen-1-yl)- | | |
| 183. | 17283-81-7 | 2-Butanone, 4- | Dihydro Beta | 0.01020000 |
| | | (2,6,6-trimethyl-1- | Ionone | |
| | | cyclohexen-1-yl)- | | |
| 184. | 1885-38-7 | 2-Propenenitrile, 3- | Cinnamalva | 0.01010000 |
| | | phenyl-, (2E)- | | |
| 185. | 103-48-0 | Propanoic acid, 2- | Phenyl Ethyl | 0.00994000 |
| | | methyl-, 2- | Iso Butyrate | |
| | | phenylethyl ester | | |
| 186. | 488-10-8 | 2-Cyclopenten-1- | Cis Jasmone | 0.00982000 |
| | | one, 3-methyl-2- | | |
| | | (2Z)-2-penten-1-yl- | | |
| 187. | 7492-67-3 | Acetaldehyde, 2-[(3, | Citronellyloxy- | 0.00967000 |
| | | 7-dimethyl-6-octen- | acetaldehyde | |
| | | 1-yl)oxy]- | | |
| 188. | 68683-20-5 | 1-Cyclohexene-1- | Iso Bergamate | 0.00965000 |
| | | ethanol, 4-(1- | | |
| | | methylethyl)-, 1- | | |
| | | formate | | |
| 189. | 3025-30-7 | 2,4-Decadienoic acid, | Ethyl 2,4- | 0.00954000 |
| | | ethyl ester, (2E,4Z)- | Decadienoate | |
| 190. | 103-54-8 | 2-Propen-1-ol, 3- | Cinnamyl | 0.00940000 |
| | | phenyl-, 1-acetate | Acetate | |
| 191. | 18127-01-0 | Benzenepropanal, 4- | Bourgeonal | 0.00934000 |
| | | (1,1-dimethylethyl)- | | |
| 192. | 3738-00-9 | Naphtho[2,1-b]furan, | Ambrox ® or | 0.00934000 |
| | | dodecahydro- | Cetalox ® or | |
| | | 3a,6,6,9a-tetramethyl- | Synambran | |
| 193. | 51519-65-4 | 1,4- | Tamisone | 0.00932000 |
| | | Methanonaphthalen- | | |
| | | 5(1H)-one, | | |
| | | 4,4a,6,7,8,8a- | | |
| | | hexahydro- | | |
| 194. | 148-05-1 | Dodecanoic acid, | Dodecalactone | 0.00931000 |
| | | 12-hydroxy-, | | |
| | | λ-lactone (6CI,7CI); | | |
| | | 1,12- | | |
| 195. | 6790-58-5 | (3aR,5aS,9aS,9bR)- | Ambronat ® or | 0.00930000 |
| | | 3a,6,6,9a-tetramethyl- | Ambroxan ® | |
| | | 2,4,5,5a,7,8,9,9b- | | |
| | | octahydro-1H-benzo | | |
| | | [e][1]benzofuran | | |
| 196. | 86-26-0 | 1,1′-Biphenyl, 2- | Methyl | 0.00928000 |
| | | methoxy- | Diphenyl | |
| | | | Ether | |
| 197. | 68738-94-3 | 2-Naphthalene- | Cyclomyral ® | 0.00920000 |
| | | carboxaldehyde, | | |
| | | octahydro-8,8- | | |
| | | dimethyl | | |
| 198. | 2705-87-5 | Cyclohexane- | Allyl | 0.00925000 |
| | | propanoic acid, 2- | Cyclohexane | |
| | | propen-1-yl ester | Propionate | |
| 199. | 7011-83-8 | 2(3H)-Furanone, 5- | Lacto- | 0.00885000 |
| | | hexyldihydro-5- | jasmone ® | |
| | | methyl- | | |
| 200. | 61792-11-8 | 2,6-Nonadienenitrile, | Lemonile ® | 0.00884000 |
| | | 3,7-dimethyl- | | |
| 201. | 692-86-4 | 10-Undecenoic acid, | Ethyl | 0.00882000 |
| | | ethyl ester | Undecylenate | |
| 202. | 103-95-7 | Benzenepropanal, α- | Cymal | 0.00881000 |
| | | methyl-4-(1- | | |
| | | methylethyl)- | | |
| 203. | 13019-22-2 | 9-Decen-1-ol | Rosalva | 0.00879000 |
| 204. | 94201-19-1 | 1-Oxaspiro[4.5] | Methyl | 0.00872000 |
| | | decan-2-one, 8- | Laitone | |
| | | methyl- | 10% TEC | |
| 205. | 104-61-0 | 2(3H)-Furanone, | γ-Nonalactone | 0.00858000 |
| | | dihydro-5-pentyl- | | |
| 206. | 706-14-9 | 2(3H)-Furanone, 5- | γ-Decalactone | 0.00852000 |
| | | hexyldihydro- | | |
| 207. | 24720-09-0 | 2-Buten-1-one, 1- | α-Damascone | 0.00830000 |
| | | (2,6,6-trimethyl-2- | | |
| | | cyclohexen-1-yl)-, | | |
| | | (2E)- | | |
| 208. | 39872-57-6 | 2-Buten-1-one, 1- | Isodamascone | 0.00830000 |
| | | (2,4,4-trimethyl-2- | | |
| | | cyclohexen-1-yl)-, | | |
| | | (2E)- | | |
| 209. | 705-86-2 | 2H-Pyran-2-one, | Decalactone | 0.00825000 |
| | | tetrahydro-6-pentyl- | | |
| 210. | 67634-15-5 | Benzenepropanal, 4- | Floralozone | 0.00808000 |
| | | ethyl-α,α-dimethyl- | | |
| 211. | 40527-42-2 | 1,3-Benzodioxole, 5- | Heliotropin | 0.00796000 |
| | | (diethoxymethyl)- | Diethyl Acetal | |
| 212. | 56973-85-4 | 4-Penten-1-one, 1- | Neobutenone | 0.00763000 |
| | | (5,5-dimethyl-1- | α | |
| | | cyclohexen-1-yl)- | | |
| 213. | 128-51-8 | Bicyclo[3.1.1]hept- | Nopyl | 0.00751000 |
| | | 2-ene-2-ethanol, 6,6- | Acetate | |
| | | dimethyl-, 2-acetate | | |
| 214. | 103-36-6 | 2-Propenoic acid, 3- | Ethyl | 0.00729000 |
| | | phenyl-, ethyl ester | Cinnamate | |
| 215. | 5182-36-5 | 1,3-Dioxane, 2,4,6- | Floropal ® | 0.00709000 |
| | | trimethyl-4-phenyl- | | |
| 216. | 42604-12-6 | Cyclododecane, | Boisambrene | 0.00686000 |
| | | (methoxymethoxy)- | | |
| 217. | 33885-52-8 | Bicyclo[3.1.1]hept- | Pinyl Iso | 0.00685000 |
| | | 2-ene-2-propanal, | Butyrate | |
| | | α,α,6,6-tetramethyl- | Alpha | |
| 218. | 92015-65-1 | 2(3H)-Benzofuranone, | Natactone | 0.00680000 |
| | | hexahydro-3,6- | | |
| | | dimethyl- | | |
| 219. | 63767-86-2 | Cyclohexanemethanol, | Mugetanol | 0.00678000 |
| | | α-methyl-4-(1- | | |
| | | methylethyl)- | | |
| 220. | 3288-99-1 | Benzeneacetonitrile, | Marenil CI | 0.00665000 |
| | | 4-(1,1-dimethylethyl)- | | |
| 221. | 35044-68-9 | 2-Buten-1-one, 1- | beta- | 0.00655000 |
| | | (2,6,6-trimethyl-1- | Damascone | |
| | | cyclohexen-1-yl)- | | |
| 222. | 41724-19-0 | 1,4- | Plicatone | 0.00652000 |
| | | Methanonaphthalen- | | |
| | | 6(2H)-one, | | |
| | | octahydro-7-methyl | | |
| 223. | 75147-23-8 | Bicyclo[3.2.1]octan- | Buccoxime ® | 0.00647000 |
| | | 8-one, 1,5-dimethyl-, | | |
| | | oxime | | |
| 224. | 25634-93-9 | 2-Methyl-5- | Rosaphen ® | 0.00637000 |
| | | phenylpentan-1-ol | 600064 | |
| 225. | 55066-48-3 | 3-Methyl-5- | Phenyl | 0.00637000 |
| | | phenylpentanol | Hexanol | |
| 226. | 495-62-5 | Cyclohexene, 4-(1,5- | Bisabolene | 0.00630000 |
| | | dimethyl-4-hexen-1- | | |
| | | ylidene)-1-methyl- | | |
| 227. | 2785-87-7 | Phenol, 2-methoxy- | Dihydro | 0.00624000 |
| | | 4-propyl- | Eugenol | |
| 228. | 87-19-4 | Benzoic acid, 2- | Iso Butyl | 0.00613000 |
| | | hydroxy-, 2- | Salicylate | |
| | | methylpropyl ester | | |
| 229. | 4430-31-3 | 2H-1-Benzopyran-2- | Octahydro | 0.00586000 |
| | | one, octahydro- | Coumarin | |
| 230. | 38462-22-5 | Cyclohexanone, 2- | Ringonol 50 | 0.00585000 |
| | | (1-mercapto-1- | TEC | |
| | | methylethyl)-5- | | |
| | | methyl- | | |
| 231. | 77-83-8 | 2-Oxiranecarboxylic | Ethyl Methyl | 0.00571000 |
| | | acid, 3-methyl-3- | Phenyl | |
| | | phenyl-, ethyl ester | Glycidate | |
| 232. | 37677-14-8 | 3-Cyclohexene-1- | Iso Hexenyl | 0.00565000 |
| | | carboxaldehyde, 4- | Cyclohexenyl | |
| | | (4-methyl-3-penten- | Carboxalde- | |
| | | 1-yl)- | hyde | |
| 233. | 103-60-6 | Propanoic acid, 2- | Phenoxy | 0.00562000 |
| | | methyl-, 2- | Ethyl iso- | |
| | | phenoxyethyl ester | Butyrate | |
| 234. | 18096-62-3 | Indeno[1,2-d]-1,3- | Indoflor ® | 0.00557000 |
| | | dioxin, 4,4a, 5,9b- | | |
| | | tetrahydro- | | |
| 235. | 63500-71-0 | 2H-Pyran-4-ol, | Florosa Q/ | 0.00557000 |
| | | tetrahydro-4-methyl- | Florol | |
| | | 2-(2-methylpropyl)- | | |
| 236. | 65405-84-7 | Cyclohexanebutanal, | Cetonal ® | 0.00533000 |
| | | α,2,6,6-tetramethyl- | | |
| 237. | 171102-41-3 | 4,7-Methano- | Flor Acetate | 0.00530000 |
| | | 1H-inden-6-ol, | | |
| | | 3a,4,5,6,7,7a- | | |
| | | hexahydro-8,8- | | |
| | | dimethyl-, 6-acetate | | |
| 238. | 10339-55-6 | 1,6-Nonadien-3-ol, | Ethyl linalool | 0.00520000 |
| | | 3,7-dimethyl- | | |
| 239. | 23267-57-4 | 3-Buten-2-one, 4- | Ionone | 0.00520000 |
| | | (2,2,6-trimethyl-7- | Epoxide Beta | |
| | | oxabicyclo[4.1.0] | | |
| | | hept-1-yl)- | | |
| 240. | 97-54-1 | Phenol, 2-methoxy- | Isoeugenol | 0.00519000 |
| | | 4-(1-propen-1-yl)- | | |
| 241. | 67663-01-8 | 2(3H)-Furanone, 5- | Peacholide | 0.00512000 |
| | | hexyldihydro-4- | | |
| | | methyl- | | |
| 242. | 33885-52-8 | Bicyclo[3.1.1]hept- | Pinyl Iso | 0.00512000 |
| | | 2-ene-2-propanal, | Butyrate | |
| | | α,α,6,6-tetramethyl- | Alpha | |
| 243. | 23696-85-7 | 2-Buten-1-one, 1- | Damascenone | 0.00503000 |
| | | (2,6,6-trimethyl-1,3- | | |
| | | cyclohexadien-1-yl)- | | |
| 244. | 80-71-7 | 2-Cyclopenten-1- | Maple | 0.00484000 |
| | | one, 2-hydroxy-3- | Lactone | |
| | | methyl- | | |
| 245. | 67662-96-8 | Propanoic acid, 2,2- | Pivarose Q | 0.00484000 |
| | | dimethyl-, 2- | | |
| | | phenylethyl ester | | |
| 246. | 2437-25-4 | Dodecanenitrile | Clonal | 0.00480000 |
| 247. | 141-14-0 | 6-Octen-1-ol, 3,7- | Citronellyl | 0.00469000 |
| | | dimethyl-, 1- | Propionate | |
| | | propanoate | | |
| 248. | 54992-90-4 | 3-Buten-2-one, 4- | Myrrhone | 0.00460000 |
| | | (2,2,3,6-tetramethyl- | | |
| | | cyclohexyl)- | | |
| 249. | 55066-49-4 | Benzenepentanal, β- | Mefranal | 0.00455000 |
| | | methyl- | | |
| 250. | 7493-74-5 | Acetic acid, 2- | Allyl Phenoxy | 0.00454000 |
| | | phenoxy-, 2-propen- | Acetate | |
| | | 1-yl ester | | |
| 251. | 80-54-6 | Benzenepropanal, 4- | Lilial ® | 0.00444000 |
| | | (1,1-dimethylethyl)- | | |
| | | α-methyl- | | |
| 252. | 86803-90-9 | 4,7-Methano-1H- | Scentenal ® | 0.00439000 |
| | | indene-2- | | |
| | | carboxaldehyde, | | |
| | | octahydro-5-methoxy- | | |
| 253. | 68991-97-9 | 2-Naphthalenecar- | Melafleur | 0.00436000 |
| | | boxaldehyde, | | |
| | | 1,2,3,4,5,6,7,8- | | |
| | | octahydro-8,8- | | |
| | | dimethyl- | | |
| 254. | 18871-14-2 | Pentitol, 1,5-anhydro- | Jasmal | 0.00434000 |
| | | 2,4-dideoxy-2-pentyl-, | | |
| | | 3-acetate | | |
| 255. | 58567-11-6 | Cyclododecane, | Boisambren | 0.00433000 |
| | | (ethoxymethoxy)- | Forte | |
| 256. | 94400-98-3 | Naphth[2,3-b]oxirene, | Molaxone | 0.00425000 |
| | | 1a,2,3,4,5,6,7,7a- | | |
| | | octahydro-1a, | | |
| | | 3,3,4,6,6-hexamethyl-, | | |
| | | (1aR,4S,7aS)-rel- | | |
| 257. | 79-69-6 | 3-Buten-2-one, 4- | alpha-Irone | 0.00419000 |
| | | (2,5,6,6-tetramethyl- | | |
| | | 2-cyclohexen-1-yl)- | | |
| 258. | 65442-31-1 | Quinoline, 6-(1- | Iso Butyl | 0.00408000 |
| | | methylpropyl)- | Quinoline | |
| 259. | 87731-18-8 | Carbonic acid, 4- | Violiff | 0.00401000 |
| | | cycloocten-1-yl | | |
| | | methyl ester | | |
| 260. | 173445-65-3 | 1H-Indene-5- | Hivernal | 0.00392000 |
| | | propanal, 2,3- | (A-isomer) | |
| | | dihydro-3,3- | | |
| | | dimethyl- | | |
| 261. | 23911-56-0 | Ethanone, 1-(3- | Nerolione | 0.00383000 |
| | | methyl-2- | | |
| | | benzofuranyl)- | | |
| 262. | 52474-60-9 | 3-Cyclohexene-1- | Precyclemone | 0.00381000 |
| | | carboxaldehyde, 1- | B | |
| | | methyl-3-(4-methyl- | | |
| | | 3-penten-1-yl)- | | |
| 263. | 139539-66-5 | 6-Oxabicyclo | Cassifix | 0.00381000 |
| | | [3.2.1]octane, 5- | | |
| | | methyl-1-(2,2,3- | | |
| | | trimethyl-3- | | |
| | | cyclopenten-1-yl)- | | |
| 264. | 80858-47-5 | Benzene, [2- | Phenafleur | 0.00380000 |
| | | (cyclohexyloxy) | | |
| | | ethyl]- | | |
| 265. | 32764-98-0 | 2H-Pyran-2-one, | Jasmolactone | 0.00355000 |
| | | tetrahydro-6-(3- | | |
| | | penten-1-yl)- | | |
| 266. | 78417-28-4 | 2,4,7-Decatrienoic | Ethyl 2,4,7- | 0.00353000 |
| | | acid, ethyl ester | decatrienoate | |
| 267. | 140-26-1 | Butanoic acid, 3- | Beta Phenyl | 0.00347000 |
| | | methyl-, 2- | Ethyl | |
| | | phenylethyl ester | Isovalerate | |
| 268. | 105-90-8 | 2,6-Octadien-1-ol, | Geranyl | 0.003360000 |
| | | 3,7-dimethyl-, 1- | Propionate | |
| | | propanoate, (2E)- | | |
| 269. | 41816-03-9 | Spiro[1,4- | Rhubofix ® | 0.00332000 |
| | | methanonaphthalene- | | |
| | | 2(1H),2′-oxirane], | | |
| | | 3,4,4a,5,8,8a- | | |
| | | hexahydro- | | |
| | | 3′,7-dimethyl- | | |
| 270. | 7070-15-7 | Ethanol, 2- | Arbanol | 0.00326000 |
| | | [[(1R,2R,4R)-1,7,7- | | |
| | | trimethylbicyclo | | |
| | | [2.2.1]hept-2-yl]oxy]-, | | |
| | | rel- | | |
| 271. | 93-29-8 | Phenol, 2-methoxy- | Iso Eugenol | 0.00324000 |
| | | 4-(1-propen-1-yl)-, | Acetate | |
| | | 1-acetate | | |
| 272. | 476332-65-7 | 2H-Indeno[4,5- | Amber | 0.00323000 |
| | | b]furan, decahydro- | Xtreme | |
| | | 2,2,6,6,7,8,8- | Compound 1 | |
| | | heptamethyl- | | |
| 273. | 68901-15-5 | Acetic acid, 2- | Cyclo- | 0.00323000 |
| | | (cyclohexyloxy)-, 2- | galbanate | |
| | | propen-1-yl ester | | |
| 274. | 107-75-5 | Octanal, 7-hydroxy- | Hydroxy- | 0.00318000 |
| | | 3,7-dimethyl- | citronellal | |
| 275. | 68611-23-4 | Naphtho[2,1-b]furan, | Grisalva | 0.00305000 |
| | | 9b-ethyldodecahydro- | | |
| | | 3a,7,7-trimethyl- | | |
| 276. | 313973-37-4 | 1,6-Heptadien-3- | Pharaone | 0.00298000 |
| | | one, 2-cyclohexyl- | | |
| 277. | 137-00-8 | 5-Thiazoleethanol, | Sulfurol | 0.00297000 |
| | | 4-methyl- | | |
| 278. | 7779-30-8 | 1-Penten-3-one, 1- | Methyl Ionone | 0.00286000 |
| | | (2,6,6-trimethyl-2- | | |
| | | cyclohexen-1-yl)- | | |
| 279. | 127-51-5 | 3-Buten-2-one, 3- | Isoraldeine | 0.00282000 |
| | | methyl-4-(2,6,6- | Pure | |
| | | trimethyl-2- | | |
| | | cyclohexen-1-yl)- | | |
| 280. | 72903-27-6 | 1,4- | Fructalate ™ | 0.00274000 |
| | | Cyclohexane- | | |
| | | dicarboxylic acid, | | |
| | | 1,4-diethyl ester | | |
| 281. | 7388-22-9 | 3-Buten-2-one, 4- | Ionone | 0.00272000 |
| | | (2,2-dimethyl-6- | Gamma | |
| | | methylenecyclohexy | Methyl | |
| | | 1)-3-methyl- | | |
| 282. | 104-67-6 | 2(3H)-Furanone, 5- | gamma- | 0.00271000 |
| | | heptyldihydro- | Undecalactone | |
| | | | (racemic) | |
| 283. | 1205-17-0 | 1,3-Benzodioxole-5- | Helional | 0.00270000 |
| | | propanal, α-methyl- | | |
| 284. | 33704-61-9 | 4H-Inden-4-one, | Cashmeran | 0.00269000 |
| | | 1,2,3,5,6,7- | | |
| | | hexahydro-1,1,2,3,3- | | |
| | | pentamethyl- | | |
| 285. | 36306-87-3 | Cyclohexanone, 4- | Kephalis | 0.00269000 |
| | | (1-ethoxyethenyl)- | | |
| | | 3,3,5,5-tetramethyl- | | |
| 286. | 97384-48-0 | Benzene- | Citrowanil ® | 0.00265000 |
| | | propanenitrile, | B | |
| | | α-ethenyl-α-methyl- | | |
| 287. | 141-13-9 | 9-Undecenal, 2,6, | Adoxal | 0.00257000 |
| | | 10-trimethyl- | | |
| 288. | 2110-18-1 | Pyridine, 2-(3- | Corps Racine | 0.00257000 |
| | | phenylpropyl)- | VS | |
| 289. | 27606-09-3 | Indeno[1,2-d]-1,3- | Magnolan | 0.00251000 |
| | | dioxin, 4,4a,5,9b- | | |
| | | tetrahydro-2,4- | | |
| | | dimethyl- | | |
| | 57082-24-3 | Caryophyllene | Caryophyllene | 0.00025000 |
| | | acetate | acetate | |
| 290. | 67634-20-2 | Propanoic acid, 2- | Cyclabute | 0.00244000 |
| | | methyl-,3a,4,5,6,7,7a- | | |
| | | hexahydro-4,7- | | |
| | | methano-1H-inden- | | |
| | | 5-yl ester | | |
| 291. | 65405-72-3 | 1-Naphthalenol, | Oxyoctaline | 0.00236000 |
| | | 1,2,3,4,4a,7,8,8a- | Formate | |
| | | octahydro-2,4a,5,8a- | | |
| | | tetramethyl-, 1- | | |
| | | formate | | |
| 292. | 122-40-7 | Heptanal, 2- | Amyl | 0.00233000 |
| | | | Cinnamic | |
| | | (phenylmethylene)- | Aldehyde | |
| 293. | 103694-68-4 | Benzenepropanol, | Majantol ® | 0.00224000 |
| | | β,β,3-trimethyl- | | |
| 294. | 13215-88-8 | 2-Cyclohexen-1-one, | Tabanone | 0.00223000 |
| | | 4-(2-buten-1- | Coeur | |
| | | ylidene)-3,5,5- | | |
| | | trimethyl- | | |
| 295. | 25152-85-6 | 3-Hexen-1-ol, 1- | Cis-3-Hexenyl | 0.00203000 |
| | | benzoate, (3Z)- | Benzoate | |
| 296. | 406488-30-0 | 2-Ethyl-N-methyl-N- | Paradisamide | 0.00200000 |
| | | (m-tolyl)butanamide | | |
| 297. | 121-33-5 | Benzaldehyde, 4- | Vanillin | 0.00194000 |
| | | hydroxy-3-methoxy- | | |
| 298. | 77-54-3 | 1H-3a,7- | Cedac | 0.00192000 |
| | | Methanoazulen-6-ol, | | |
| | | octahydro-3,6,8,8- | | |
| | | tetramethyl-, 6- | | |
| | | acetate, | | |
| | | (3R,3aS,6R,7R,8aS)- | | |
| 299. | 76842-49-4 | 4,7-Methano-1H- | Frutene | 0.00184000 |
| | | inden-6-ol, | | |
| | | 3a,4,5,6,7,7a- | | |
| | | hexahydro-8,8- | | |
| | | dimethyl-, 6- | | |
| | | propanoate | | |
| 300. | 121-39-1 | 2-Oxiranecarboxylic | Ethyl Phenyl | 0.00184000 |
| | | acid, 3-phenyl-, | Glycidate | |
| | | ethyl ester | | |
| 301. | 211299-54-6 | 4H-4a,9- | Ambro- | 0.00182000 |
| | | Methanoazuleno | cenide ® | |
| | | [5,6-d]-1,3-dioxole, | | |
| | | octahydro- | | |
| | | 2,2,5,8,8,9a- | | |
| | | hexamethyl-, | | |
| | | (4aR,5R,7aS,9R)- | | |
| 302. | 285977-85-7 | (2,5-Dimethyl-1,3- | Lilyflore | 0.00180000 |
| | | dihydroinden-2- | | |
| | | yl)methanol | | |
| 303. | 10094-34-5 | Butanoic acid, 1,1- | Dimethyl | 0.00168000 |
| | | dimethyl-2- | Benzyl | |
| | | phenylethyl ester | Carbinyl | |
| | | | Butyrate | |
| 304. | 40785-62-4 | Cyclododeca[c] | Muscogene | 0.00163000 |
| | | furan, 1,3,3a,4,5,6,7, | | |
| | | 8,9,10,11,13a- | | |
| | | dodecahydro- | | |
| 305. | 75490-39-0 | Benzenebutanenitrile, | Khusinil | 0.00162000 |
| | | α,α,γ-trimethyl- | | |
| 306. | 55418-52-5 | 2-Butanone, 4-(1,3- | Dulcinyl | 0.00161000 |
| | | benzodioxol-5-yl)- | | |
| 307. | 3943-74-6 | Benzoic acid, 4- | Carnaline | 0.00157000 |
| | | hydroxy-3-methoxy-, | | |
| | | methyl ester | | |
| 308. | 72089-08-8 | 3-Cyclopentene-1- | Brahmanol ® | 0.00154000 |
| | | butanol, β,2,2,3- | | |
| | | tetramethyl-2-Methyl- | | |
| | | 4-(2,2,3-trimethyl-3- | | |
| | | cyclopenten-1-yl) | | |
| | | butanol | | |
| 309. | 3155-71-3 | 2-Butenal, 2-methyl- | Boronal | 0.00147000 |
| | | 4-(2,6,6-trimethyl-1- | | |
| | | cyclohexen-1-yl)- | | |
| 310. | 2050-08-0 | Benzoic acid, 2- | Amyl | 0.00144000 |
| | | hydroxy-, pentyl | Salicylate | |
| | | ester | | |
| 311. | 41199-20-6 | 2-Naphthalenol, | Ambrinol | 0.00140000 |
| | | decahydro-2,5,5- | | |
| | | trimethyl- | | |
| 312. | 12262-03-2 | ndecanoic acid, 3- | Iso Amyl | 0.00140000 |
| | | methylbutyl ester | Undecylenate | |
| 313. | 107-74-4 | 1,7-Octanediol, 3,7- | Hydroxyol | 0.00139000 |
| | | dimethyl- | | |
| 314. | 91-64-5 | 2H-1-Benzopyran-2- | Coumarin | 0.00130000 |
| | | one | | |
| 315. | 68901-32-6 | 1,3-Dioxolane, 2- | Glycolierral | 0.00121000 |
| | | [6-methyl-8-(1- | | |
| | | methylethyl)bicyclo | | |
| | | [2.2.2]oct-5-en-2-yl]- | | |
| 316. | 68039-44-1 | Propanoic acid, 2,2- | Pivacyclene | 0.00119000 |
| | | dimethyl-, 3a,4,5,6, | | |
| | | 7,7a-hexahydro-4,7- | | |
| | | methano-1H-inden- | | |
| | | 6-yl ester | | |
| 317. | 106-29-6 | Butanoic acid, (2E)- | Geranyl | 0.00116000 |
| | | 3,7-dimethyl-2,6- | Butyrate | |
| | | octadien-1-yl ester | | |
| 318. | 5471-51-2 | 2-Butanone, 4-(4- | Raspberry | 0.00106000 |
| | | hydroxyphenyl)- | ketone | |
| 319. | 109-42-2 | 10-Undecenoic acid, | Butyl | 0.00104000 |
| | | butyl ester | Undecylenate | |
| 320. | 2785-89-9 | 4-Ethyl-2- | 4- | 0.02000000 |
| | | methoxyphenol | Ethylguaiacol | |
| | 27538-10-9 | 2-ethyl-4-hydroxy- | Homofuronol | 0.01210000 |
| | | 5-methylfuran-3-one | | |

*Vapor Pressures are acquired as described in the Test Methods Section.
**Origin: Same as for Table 1 hereinabove.

TABLE 2B
Moderate Volatile Natural Oils.

| No. | Natural oil | Supplier |
|---|---|---|
| 1. | Bay Oil Terpeneless | IFF |
| 2. | Cade Oil | H. Reynaud & Fils |
| 3. | Cedar Atlas Oil | Robertet |
| 4. | Cinnamon Bark Oil | Robertet |
| 5. | Cinnamon Oleoresin | Citrus & Allied Essences |
| 6. | Clove Bud Oil | Robertet |
| 7. | Clove Leaf Oil Rectified | H. Reynaud & Fils |
| 8. | Clove Stem Oil | H. Reynaud & Fils |
| 9. | Davana Oil | Robertet |
| 10. | Geranium Bourbon | Robertet |
| 11. | Ginger Oil Fresh Madagascar | IFF |
| 12. | Hay Absolute MD 50 PCT | IFF |
| 13. | Juniperberry Oil T'less | Robertet |
| 14. | Papyrus Oil | Robertet |
| 15. | Rose Absolute Oil | Robertet |
| 16. | Tonka Bean Absolute | Robertet |
| 17. | Wormwood Oil | Robertet |

### Suppliers:

Citrus & Allied Essences, New York, USA
H. Reynaud & Fils, Montbrun-les-Bains, France
IFF, Hazlet, New Jersey, USA
Robertet, Grasse, France

Moderate volatile fragrance materials can selected from the group of Tables 2A or 2B. However, it is understood by one skilled in the art that other moderate volatile fragrance materials, not recited in Tables 2A or 2B, would also fall within the scope of the present invention, so long as they have a vapor pressure of 0.1 to 0.001 Torr at 25° C.

(iii) High Volatile Fragrance Materials

The fragrance component includes at least one high volatile fragrance material having a vapor pressure greater than 0.1 Torr (0.0133 kPa) at 25° C. In some examples, the high volatile fragrance material can include at least 2 high volatile fragrance materials, 3 high volatile fragrance materials, or at least 5 high volatile fragrance materials, or at least 7 high volatile fragrance materials. If there are more than one high volatile fragrance materials, then the ranges provided hereinabove cover the total of all of the high volatile fragrance materials. Suitable examples of high volatile fragrances materials are provided in Tables 3A and 3B below.

Preferably, the high volatile fragrance material is selected from at least 1 material, or at least 2 materials, or at least 3 materials, or at least 5 materials, at least 7 materials, or at least 9 high volatile fragrance materials as disclosed in Table 3A. Natural fragrance materials or oils having an aggregate vapour pressure greater than 0.1 Torr (0.0133 kPa) at 25° C. are provided in Table 3B. Moderate Volatile Natural Oils.

TABLE 3A
High Volatile Fragrance Materials

| | | | | Vapor |
|---|---|---|---|---|
| | | | | Pressure |
| | CAS | IUPAC | Common | (Torr |
| No. | Number | Name | Name** | at 25° C.)* |
| 1. | 107- | Formic acid, | Methyl | 732.00000000 |
| | 31-3 | methyl ester | Formate | |
| 2. | 75- | Methane, 1,1′- | Dimethyl | 647.00000000 |
| | 18-3 | thiobis- | Sulfide | |
| | | | 1.0% In DEP | |
| 3. | 141- | Acetic acid | Ethyl Acetate | 112.00000000 |
| | 78-6 | ethyl ester | | |
| 4. | 105- | Propanoic acid, | Ethyl | 44.50000000 |
| | 37-3 | ethyl ester | Propionate | |
| 5. | 110- | Acetic acid, 2- | Isobutyl | 18.00000000 |
| | 19-0 | methylpropyl | Acetate | |
| | | ester | | |
| 6. | 105- | Butanoic acid, | Ethyl Butyrate | 13.90000000 |
| | 54-4 | ethyl ester | | |
| 7. | 14765- | 1-Butanol | Butyl Alcohol | 8.52000000 |
| | 30-1 | | | |
| 8. | 7452- | Butanoic acid, | Ethyl-2-Methyl | 7.85000000 |
| | 79-1 | 2-methyl-, | Butyrate | |
| | | ethyl ester | | |
| 9. | 123- | 1-Butanol, 3- | Iso Amyl | 5.68000000 |
| | 92-2 | methyl-, 1- | Acetate | |
| | | acetate | | |
| 10. | 66576- | Butanoic acid, | Iso Propyl 2- | 5.10000000 |
| | 71-4 | 2-methyl-, 1- | Methylbutyrate | |
| | | methylethyl | | |
| | | ester | | |
| 11. | 110- | 2-Heptanone | Methyl Amyl | 4.73000000 |
| | 43-0 | | Ketone | |
| 12. | 6728- | 2-Hexenal, | Trans-2 | 4.62000000 |
| | 26-3 | (2E)- | Hexenal | |
| 13. | 123-51-3 | 1-Butanol, 3- | Isoamyl | 4.16000000 |
| | | methyl- | Alcohol | |
| 14. | 1191- | 2-Buten-1-ol, | Prenyl acetate | 3.99000000 |
| | 16-8 | 3-methyl-, | | |
| | | 1-acetate | | |
| 15. | 57366- | 1,3-Dioxolane- | Methyl | 3.88000000 |
| | 77-5 | 2-methanamine, | Dioxolan | |
| | | N-methyl- | | |
| 16. | 7785- | Bicyclo[3.1.1] | Alpha Pinene | 3.49000000 |
| | 70-8 | hept-2-ene, 2,6, | | |
| | | 6-trimethyl-, | | |
| | | (1R,5R)- | | |
| 17. | 79- | Bicyclo[2.2.1] | Camphene | 3.38000000 |
| | 92-5 | heptane, 2,2- | | |
| | | dimethyl-3- | | |
| | | methylene- | | |
| 18. | 94087- | 2-Butanethiol, | 4-Methoxy-2- | 3.31000000 |
| | 83-9 | 4-methoxy-2- | Methyl-2- | |
| | | methyl- | Butanenthiol | |
| 19. | 39255- | Pentanoic acid, | Manzanate | 2.91000000 |
| | 32-8 | 2-methyl-, | | |
| | | ethyl ester | | |
| 20. | 3387- | Bicyclo[3.1.0] | Sabinene | 2.63000000 |
| | 41-5 | hexane, 4- | | |
| | | methylene-1-(1- | | |
| | | methylethyl)- | | |
| 21. | 127- | Bicyclo[3.1.1] | Beta Pinene | 2.40000000 |
| | 91-3 | heptane, 6,6- | | |
| | | dimethyl-2- | | |
| | | methylene- | | |
| 22. | 105- | 1-Butanol, 3- | Amyl | 2.36000000 |
| | 68-0 | methyl-, 1- | Propionate | |
| | | propanoate | | |
| 23. | 123- | 1,6-Octadiene, | Myrcene | 2.29000000 |
| | 35-3 | 7-methyl-3- | | |
| | | methylene- | | |
| 24. | 124-13-0 | Octanal | Octyl Aldehyde | 2.07000000 |
| 25. | 7392- | 2H-Pyran, 2- | Limetol | 1.90000000 |
| | 19-0 | ethenyltetra- | | |
| | | hydro- | | |
| | | 2,6,6- | | |
| | | trimethyl- | | |
| 26. | 111- | 2-Octanone | Methyl Hexyl | 1.72000000 |
| | 13-7 | | Ketone | |
| 27. | 123- | Hexanoic acid, | Ethyl Caproate | 1.66000000 |
| | 66-0 | ethyl ester | | |
| 28. | 470- | 2-Oxa- | Eucalyptol | 1.65000000 |
| | 82-6 | bicyclo[2.2.2] | | |
| | | octane, 1,3,3- | | |
| | | trimethyl- | | |
| 29 | 99- | Benzene, 1- | Para Cymene | 1.65000000 |
| | 87-6 | methyl-4-(1- | | |
| | | methylethyl)- | | |
| 30. | 104- | Benzene, 1- | Para Cresyl | 1.65000000 |
| | 93-8 | methoxy-4- | Methyl Ether | |
| | | methyl- | | |
| 31. | 13877- | 1,3,6-Octatriene, | Ocimene | 1.56000000 |
| | 91-3 | 3,7-dimethyl- | | |
| 32. | 138- | Cyclohexene, 1- | dl-Limonene | 1.54000000 |
| | 86-3 | methyl-4-(1- | | |
| | | methylethenyl)- | | |
| 33. | 5989- | Cyclohexene, 1- | d-limonene | 1.54000000 |
| | 27-5 | methyl-4-(1- | | |
| | | methylethenyl)-, | | |
| | | (4R)- | | |
| 34. | 106- | 3-Octanone | Ethyl Amyl | 1.50000000 |
| | 68-3 | | Ketone | |
| 35. | 110- | Undecanal, 2- | Methyl Nonyl | 1.43000000 |
| | 41-8 | methyl- | Acetaldehyde | |
| 36. | 142- | Acetic acid, | Hexyl acetate | 1.39000000 |
| | 92-7 | hexyl ester | | |
| 37. | 110- | 5-Hepten-2-one, | Methyl | 1.28000000 |
| | 93-0 | 6-methyl- | Heptenone | |
| 38. | 81925- | 2-Hepten-4-one, | Filbertone | 1.25000000 |
| | 81-7 | 5-methyl- | 1% in TEC | |
| 39. | 3681- | 3-Hexen-1-ol, | cis-3-Hexenyl | 1.22000000 |
| | 71-8 | 1-acetate, (3Z)- | acetate | |
| 40. | 97- | Propanoic acid, | Ethyl Lactate | 1.16000000 |
| | 64-3 | 2-hydroxy-, | | |
| | | ethyl ester | | |
| 41. | 586- | Cyclohexene, | Terpineolene | 1.13000000 |
| | 62-9 | 1-methyl-4-(1- | | |
| | | methyl- | | |
| | | ethylidene)- | | |
| 42. | 51115- | Butanoic acid, | Amyl butyrate | 1.09000000 |
| | 64-1 | 2-methylbutyl | | |
| | | ester | | |
| 43. | 106- | Butanoic acid, | Amyl Butyrate | 1.09000000 |
| | 27-4 | 3-methylbutyl | | |
| | | ester | | |
| 44. | 99- | 1,4- | Gamma | 1.08000000 |
| | 85-4 | Cyclohexadiene, | Terpinene | |
| | | 1-methyl-4-(1- | | |
| | | methylethyl)- | | |
| 45. | 18640- | Thiazole, 2-(2- | 2-Isobutyl- | 1.07000000 |
| | 74-9 | methylpropyl)- | thiazole | |
| 46. | 928- | 3-Hexen-1-ol, | cis-3-Hexenol | 1.04000000 |
| | 96-1 | (3Z)- | | |
| 47. | 100- | Benzaldehyde | Benzaldehyde | 0.97400000 |
| | 52-7 | | | |
| 48. | 141- | Butanoic acid, | Ethyl | 0.89000000 |
| | 97-9 | 3-oxo-, | Acetoacetate | |
| | | ethyl ester | | |
| 49. | 928- | 2-Hexen-1-ol, | Trans-2- | 0.87300000 |
| | 95-0 | (2E)- | Hexenol | |
| 50. | 928- | 2-Hexen-1-ol, | Beta Gamma | 0.87300000 |
| | 94-9 | (2Z)- | Hexenol | |
| 51. | 24691- | Cyclohexane, | Herbavert | 0.85200000 |
| | 15-4 | 3-ethoxy-1,1, | | |
| | | 5-trimethyl-, | | |
| | | cis-(9CI) | | |
| 52. | 19872- | 2-Pentanone, | 4-Methyl-4- | 0.84300000 |
| | 52-7 | 4-mercapto-4- | Mercapto- | |
| | | methyl- | pentan-2-one | |
| | | | 1 ppm TEC | |
| 53. | 3016- | 2,4,6- | Allo-Ocimene | 0.81600000 |
| | 19-1 | Octatriene, | | |
| | | 2,6-dimethyl-, | | |
| | | (4E,6E)- | | |
| 54. | 69103- | Oxirane, 2,2- | Myroxide | 0.80600000 |
| | 20-4 | dimethyl-3-(3- | | |
| | | methyl-2,4- | | |
| | | pentadien- | | |
| | | 1-yl)- | | |
| 55. | 189440- | 4,7-Octadienoic | Anapear | 0.77700000 |
| | 77-5 | acid, methyl | | |
| | | ester, (4E)- | | |
| 56. | 67633- | Carbonic acid, | Liffarome ™ | 0.72100000 |
| | 96-9 | (3Z)-3-hexen-1- | | |
| | | yl methyl ester | | |
| 57. | 123- | Hexanoic acid, | Allyl Caproate | 0.67800000 |
| | 68-2 | 2-propen-1-yl | | |
| | | ester | | |
| 58. | 106- | 5-Heptenal, | Melonal | 0.62200000 |
| | 72-9 | 2,6-dimethyl- | | |
| 59. | 106- | Heptanoic acid, | Ethyl | 0.60200000 |
| | 30-9 | ethyl ester | Oenanthate | |
| 60. | 68039- | 3-Cyclohexene- | Ligustral or | 0.57800000 |
| | 49-6 | 1- | Triplal | |
| | | carboxaldehyde, | | |
| | | 2,4-dimethyl- | | |
| 61. | 101- | Benzene, (2,2- | Phenyl | 0.55600000 |
| | 48-4 | dimethoxy- | Acetaldehyde | |
| | | ethyl)- | Dimethyl Acetal | |
| 62. | 16409- | 2H-Pyran, | Rose Oxide | 0.55100000 |
| | 43-1 | tetrahydro-4- | | |
| | | methyl-2-(2- | | |
| | | methyl-1- | | |
| | | propen-1-yl)- | | |
| 63. | 925- | 3-Nonanone | Ethyl Hexyl | 0.55100000 |
| | 78-0 | | Ketone | |
| 64. | 100- | Benzonitrile | Benzyl Nitrile | 0.52400000 |
| | 47-0 | | | |
| 65. | 589- | 3-Octanol | Octanol-3 | 0.51200000 |
| | 98-0 | | | |
| 66. | 58430- | 1-Hexanol, | Iso Nonyl | 0.47000000 |
| | 94-7 | 3,5,5-trimethyl-, | Acetate | |
| | | 1-acetate | | |
| 67. | 10250- | 4-Heptanol, | Alicate | 0.45400000 |
| | 45-0 | 2,6-dimethyl-, | | |
| | | 4-acetate | | |
| 68. | 105- | Hexanoic acid, | Iso Butyl | 0.41300000 |
| | 79-3 | 2-methylpropyl | Caproate | |
| | | ester | | |
| 69. | 2349- | Propanoic acid, | Hexyl | 0.41300000 |
| | 07-7 | 2-methyl-, | isobutyrate | |
| | | hexyl ester | | |
| 70. | 23250- | Cyclohexane- | Cyprissate | 0.40500000 |
| | 42-2 | carboxylic | | |
| | | acid, 1,4- | | |
| | | dimethyl-, | | |
| | | methyl ester, | | |
| | | trans- | | |
| 71. | 122- | Benzene- | Phenyl | 0.36800000 |
| | 78-1 | acetaldehyde | acetaldehyde | |
| 72. | 5405- | Butanoic acid, | Ethyl-3- | 0.36200000 |
| | 41-4 | 3-hydroxy-, | Hydroxy | |
| | | ethyl ester | Butyrate | |
| 73. | 105- | Propanedioic | Diethyl | 0.34400000 |
| | 53-3 | acid, 1,3-diethyl | Malonate | |
| | | ester | | |
| 74. | 93- | Benzoic acid, | Methyl Benzoate | 0.34000000 |
| | 58-3 | methyl ester | | |
| 75. | 16356- | 1,3,5- | Undecatriene | 0.33600000 |
| | 11-9 | Undecatriene | | |
| 76. | 65405- | 4-Decenal, | Decenal | 0.33100000 |
| | 70-1 | (4E)- | (Trans-4) | |
| 77. | 54546- | 1,3-Dioxane, 2- | Herboxane | 0.33000000 |
| | 26-8 | butyl-4,4,6- | | |
| | | trimethyl- | | |
| 78. | 13254- | 2-Heptanol, | Dimethyl-2 6- | 0.33000000 |
| | 34-7 | 2,6-dimethyl- | Heptan-2-ol | |
| 79. | 98- | Ethanone, 1- | Acetophenone | 0.29900000 |
| | 86-2 | phenyl- | | |
| 80. | 93- | Benzeneacetalde | Hydratropic | 0.29400000 |
| | 53-8 | hyde, α-methyl- | aldehyde | |
| 81. | 80118- | Propanoic acid, | Iso Pentyrate | 0.28500000 |
| | 06-5 | 2-methyl-, 1,3- | | |
| | | dimethyl-3- | | |
| | | buten-1-yl ester | | |
| 82. | 557- | 2,6-Nonadienal, | EZ-2,6- | 0.28000000 |
| | 48-2 | (2E,6Z)- | Nonadien-1-al | |
| 83. | 24683- | Pyrazine, 2- | 2-Methoxy-3- | 0.27300000 |
| | 00-9 | methoxy-3-(2- | Isobutyl | |
| | | methylpropyl)- | Pyrazine | |
| 84. | 104- | Formic acid, | Benzyl Formate | 0.27300000 |
| | 57-4 | phenylmethyl | | |
| | | ester | | |
| 85. | 104- | Benzene, 1- | Dihydroanethole | 0.26600000 |
| | 45-0 | methoxy-4- | | |
| | | propyl- | | |
| 86. | 491- | Cyclohexanone, | Iso Menthone | 0.25600000 |
| | 07-6 | 5-methyl-2-(1- | | |
| | | methylethyl)-, | | |
| | | (2R,5R)-rel- | | |
| 87. | 89- | Cyclohexanone, | Menthone | 0.25600000 |
| | 80-5 | 5-methyl-2-(1- | Racemic | |
| | | methylethyl)-, | | |
| | | (2R,5S)-rel- | | |
| 88. | 2463- | 2-Nonenal | 2 Nonen-1-al | 0.25600000 |
| | 53-8 | | | |
| 89. | 55739- | Cyclohexanone, | Thuyacetone | 0.25000000 |
| | 89-4 | 2-ethyl-4,4- | | |
| | | dimethyl- | | |
| 90. | 150- | Benzene, 1,4- | Hydroquinone | 0.25000000 |
| | 78-7 | dimethoxy- | Dimethyl Ether | |
| 91. | 64988- | Benzene, 1- | Rosacene | 0.24600000 |
| | 06-3 | (ethoxymethyl)- | | |
| | | 2-methoxy- | | |
| 92. | 76- | Bicyclo[2.2.1] | Camphor gum | 0.22500000 |
| | 22-2 | heptan-2-one, | | |
| | | 1,7,7-trimethyl- | | |
| 93. | 67674- | 2-Hexene, 6,6- | Methyl | 0.21400000 |
| | 46-8 | dimethoxy-2,5, | Pamplemousse | |
| | | 5-trimethyl- | | |
| 94. | 112- | Decanal | Decyl Aldehyde | 0.20700000 |
| | 31-2 | | | |
| 95. | 16251- | Benzene- | Trifernal | 0.20600000 |
| | 77-7 | propanal, | | |
| | | β-methyl- | | |
| 96. | 93- | Benzene- | Methylphenyl- | 0.20300000 |
| | 92-5 | methanol, | carbinol | |
| | | α-methyl-, 1- | Acetate | |
| | | acetate | | |
| 97. | 143- | Acetic acid, | Nonyl Acetate | 0.19700000 |
| | 13-5 | nonyl ester | | |
| 98. | 122- | Ethanone, 1-(4- | Para Methyl | 0.18700000 |
| | 00-9 | methylphenyl)- | Acetophenone | |
| 99. | 24237- | 2H-Pyran, 6- | Gyrane | 0.18600000 |
| | 00-1 | butyl-3,6- | | |
| | | dihydro-2,4- | | |
| | | dimethyl- | | |
| 100. | 41519- | Propanoic acid, | Hexenyl | 0.18200000 |
| | 23-7 | 2-methyl-, (3Z)- | Isobutyrate | |
| | | 3-hexen-1-yl | | |
| | | ester | | |
| 101. | 93- | Benzoic acid, | Ethyl Benzoate | 0.18000000 |
| | 89-0 | ethyl ester | | |
| 102. | 20780- | 3-Octanol, 3,7- | Tetrahydro | 0.18000000 |
| | 48-7 | dimethyl-, 3- | Linalyl | |
| | | acetate | Acetate | |
| 103. | 101- | Methyl 2- | Methylphenyl | 0.17600000 |
| | 41-7 | phenylacetate | acetate | |
| 104. | 40853- | 1-Hexanol, 5- | Tetrahydro | 0.17300000 |
| | 55-2 | methyl-2-(1- | Lavandulyl | |
| | | methylethyl)-, | Acetate | |
| | | 1-acetate | | |
| 105. | 933- | Cyclohexanol, | Trimethyl- | 0.17300000 |
| | 48-2 | 3,3,5-trimethyl-, | cyclohexanol | |
| | | (1R,5R)-rel- | | |
| 106. | 35158- | 2-Hexenal, 5- | Lactone of Cis | 0.17200000 |
| | 25-9 | methyl-2-(1- | Jasmone | |
| | | methylethyl)- | | |
| 107. | 18479- | 7-Octen-2-ol, | Dihydro- | 0.16600000 |
| | 58-8 | 2,6-dimethyl- | myrcenol | |
| 108. | 140- | Acetic acid, | Benzyl acetate | 0.16400000 |
| | 11-4 | phenylmethyl | | |
| | | ester | | |
| 109. | 14765- | Cyclohexanone, | 2-sec-Butyl | 0.16300000 |
| | 30-1 | 2-(1- | Cyclo | |
| | | methylpropyl)- | Hexanone | |
| 110. | 20125- | 3-Octen-1-ol, | Octenol | 0.16000000 |
| | 84-2 | (3Z)- | | |
| 111. | 142- | Heptanoic acid, | Allyl Heptoate | 0.16000000 |
| | 19-8 | 2-propen-1-yl | | |
| | | ester | | |
| 112. | 100- | Benzene- | Benzyl Alcohol | 0.15800000 |
| | 51-6 | methanol | | |
| 113. | 10032- | Butanoic acid, | Hexyl-2-Methyl | 0.15800000 |
| | 15-2 | 2-methyl-, | Butyrate | |
| | | hexyl ester | | |
| 114. | 695- | 2(3H)- | Gamma | 0.15200000 |
| | 06-7 | Furanone, | Hexalactone | |
| | | 5-ethyldihydro- | | |
| 115. | 21722- | Cyclohexane- | Cyclohexyl | 0.15200000 |
| | 83-8 | ethanol, | Ethyl | |
| | | 1-acetate | Acetate | |
| 116. | 111- | 2-Nonenoic | Methyl-2- | 0.14600000 |
| | 79-5 | acid, | | |
| | | methyl ester | Nonenoate | |
| 117. | 16491- | Butanoic acid, | Cis 3 Hexenyl | 0.13500000 |
| | 36-4 | (3Z)-3-hexen- | Butyrate | |
| | | 1-yl ester | | |
| 118. | 111- | 2-Octynoic | Methyl Heptine | 0.12500000 |
| | 12-6 | acid, | Carbonate | |
| | | methyl ester | | |
| 119. | 59323- | 1,3-Oxathiane, | Oxane | 0.12300000 |
| | 76-1 | 2-methyl-4- | | |
| | | propyl-, | | |
| | | (2R,4S)-rel- | | |
| 120. | 62439- | Heptanal, 6- | Methoxy | 0.11900000 |
| | 41-2 | methoxy-2,6- | Melonal | |
| | | dimethyl- | | |
| 121. | 13851- | Bicyclo[2.2.1] | Fenchyl | 0.11700000 |
| | 11-1 | heptan-2-ol, | Acetate | |
| | | 1,3,3-trimethyl-, | | |
| | | 2-acetate | | |
| 122. | 115- | 1,6-Octadien- | Linalyl acetate | 0.11600000 |
| | 95-7 | 3-ol, 3,7- | | |
| | | dimethyl-, | | |
| | | 3-acetate | | |
| 123. | 18479- | 2-Octanol, 2,6- | Tetra-Hydro | 0.11500000 |
| | 57-7 | dimethyl- | Myrcenol | |
| 124. | 78- | 3,7- | Tetra-Hydro | 0.11500000 |
| | 69-3 | dimethyloctan- | Linalool | |
| | | 3-ol | | |
| 125. | 111- | 1-Octanol | Octyl Alcohol | 0.11400000 |
| | 87-5 | | | |
| 126. | 71159- | 3-Cyclohexene- | Grapefruit | 0.10500000 |
| | 90-5 | 1-methanethiol, | mercaptan | |
| | | α,α,4-trimethyl- | | |
| 127. | 80- | Cyclohexane- | Menthanyl | 0.10300000 |
| | 25-1 | methanol, | Acetate | |
| | | α,α,4- | | |
| | | trimethyl-, 1- | | |
| | | acetate | | |
| 128. | 88- | Cyclohexanol, | Verdox ™ | 0.10300000 |
| | 41-5 | 2-(1,1- | | |
| | | dimethylethyl)-, | | |
| | | 1-acetate | | |
| 129. | 32210- | Cyclohexanol, | Vertenex | 0.10300000 |
| | 23-4 | 4-(1,1- | | |
| | | dimethylethyl)-, | | |
| | | 1-acetate | | |
| 130. | 112- | Undecanal | n-Undecanal | 0.10200000 |
| | 44-7 | | | |
| 131. | 124- | Nonanal | Nonanal | 0.53200000 |
| | 19-6 | | Aldehyde C-9 | |
| 132. | 929253- | 6-methoxy-2,6- | 6-methoxy-2,6- | 0.04020000 |
| | 05-4 | dimethyloctanal | dimethyl octanal | |
| 133. | 68039- | 2-propan-2- | Phenethyl | 0.24900000 |
| | 47-4 | yloxyethyl- | Isopropyl | |
| | | benzene | Ether | |
| 134. | 6413- | ethyl 2-(2- | Apple Ketal | 0.21900000 |
| | 10-1 | methyl-1,3- | | |
| | | dioxolan-2- | | |
| | | yl)acetate | | |
| 135. | 106- | 3,7- | citronellal | 0.21500000 |
| | 23-0 | dimethyloct- | | |
| | | 6-enal | | |
| 136. | 14667- | Trimethyl | Trimethyl | 1.72400000 |
| | 55-1 | Pyrazine-2,3,5 | Pyrazine-2,3,5 | |

*Vapor Pressures are acquired as described in the Test Methods Section.
**Origin: Same as for Table 1 hereinabove.

TABLE 3B
High Volatile Natural Materials

| No. | Natural oil | Supplier |
|---|---|---|
| 1. | Angelica Seeds Oil | Robertet |
| 2. | Basil Oil Grand Vert | IFF |
| 3. | Bergamot Oil Reggio Early New Crop | Capua |
| 4. | Black Pepper Oil | Robertet |
| 5. | Blackcurrant Buds Absolute | Robertet |
| 6. | Cardamom Guatamala Extract CO2 | IFF |
| 7. | Cardamom Oil Guatemala | IFF |
| 8. | Cedarleaf Oil | Kerry |
| 9. | citronella oil | H. Reynaud & Fils |
| 10. | Clary Sage Oil French | IFF |
| 11. | Coffee Extract CO2 | Firmenich |
| 12. | Cucumber Extract | Firmenich |
| 13. | Cumin Oil | Robertet |
| 14. | Cypress Oil | IFF |
| 15. | Elemi Coeur Oil | Robertet |
| 16. | Ginger oil India | IFF |
| 17. | Grapefruit Zest | Citrus & Allied Essences |
| 18. | It. Bergamot Oil | Capua |
| 19. | Labdanum Cistus Absolute | Biolandes |
| 20. | Lavandin Grosso Oil | H. Reynaud & Fils |
| 21. | Lemon Oil Winter | Capua |
| 22. | Green Mandarin Oil | Simone Gatto |
| 23. | Nutmeg Oil | Robertet |
| 24. | Oil Orange Sinensal | Citrus & Allied Essences |
| 25. | Olibanum Oil Pyrogenous | Firmenich |
| 26. | Pepper Black CO2 Oil | Firmenich |
| 27. | Petitgrain Mandarinier Oil | Misitano & Stracuzzi |
| 28. | Pink Pepper CO2 OIL | Firmenich |
| 29. | Rum CO2 Oil | Firmenich |
| 30. | Sichuan Pepper CO2 oil | Firmenich |
| 31. | Styrax Resoid | IFF |
| 32. | Tangerine Oil | Robertet |
| 33. | Thym Oil | IFF |
| 34. | Violet Leaves Absolute | Robertet |

### Suppliers

Biolandes, Le Sen, France
Capua, Campo Calabro, Italy
Citrus & Allied Essences, New York, USA
Firmenich, Geneva, Switzerland
Global Essence Inc, New Jersey, USA
H. Reynaud & Fils, Montbrun-les-Bains, France
IFF, Hazlet, New Jersey, USA
Kerry, Co. Kerry, Ireland
Mane, Le Bar-sur-Loup, France
Misitano & Stracuzzi, Messina, Italy
Robertet, Grasse, France
Simone Gatto, San Pierre Niceto, Italy

Exemplary high volatile fragrance materials selected from the group of Tables 3A or 3B are preferred. However, it is understood by one skilled in the art that other high volatile fragrance materials, not recited in Tables 3A or 3B, would also fall within the scope of the present invention, so long as they have a vapor pressure of greater than 0.1 Torr (0.0133 kPa) at 25° C.

The individual fragrance materials can be present in various concentrations of the fragrance component. For example in a “diamond construction” the low volatile material can be present in a range of from about 0 wt % to about 30 wt % of the fragrance component, about 10 wt % to about 20 wt %, less than equal to or greater than about 0 wt %, 5, 10, 15, 20, 25, or 30 wt %; the moderate volatile component can be present in a range of from about 30 wt % to about 70 wt % of the fragrance component, about 40 wt % to about 60 wt %, less than, equal to, or greater than about 30 wt %, 35, 40, 45, 50, 55, 60, 65, or about 70 wt %; the high volatile fragrance component can be present in a 0 wt % to about 30 wt % of the fragrance component, about 10 wt % to about 20 wt %, less than equal to or greater than about 0 wt %, 5, 10, 15, 20, 25, or 30 wt %.

In a “bottom heavy construction”

### (iv) Fragrance Modulators

The composition further comprises at least one modulator as described herein below. Suitable examples of the fragrance modulators include:
the compound according to Formula I:

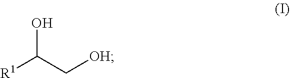

a polymer including a repeating unit derived from the compound according to Formula II:

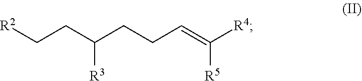

or
a mixture thereof. R¹, R², R³, R⁴, and R⁵, are independently chosen from —H, —OH, or substituted or unsubstituted (C₁-C₂₀) hydrocarbyl. In some further embodiments, R¹, R², R³, R⁴, and R⁵, are independently chosen from (C₁-C₂₀)alkyl, (C₂-C₂₀)alkenyl, (C₃-C₂₀)cycloalkyl, (C₁-C₂₀)alkoxyl, (C₁-C₂₀)aryl, or a combination thereof. In some specific embodiments, the modulator component includes pentylene glycol, polycitronellol, or a mixture thereof. In some embodiments, the polycitronellol can include 2-8 repeating units and can have a weight-average-molecular weight in a range of from about 460 g/mol to about 1500 g/mol. In some embodiments, the modulator component can include a mixture of pentylene glycol and polycitronellol and a molar ratio of pentylene glycol to polycitronellol is in a range of from about 5:1 to about 1:5, about 4:1 to about 1:4, about 3:1 to about 1:3, or about 2:1 to about 1:2, or about 1:1. In some embodiments, the modulator component can include at least some PPG-20 methyl glucose ether mixed with any of the aforementioned modulators. Alternatively, the modulator component can be free of (include 0 wt %) PPG-20 methyl glucose. If PPG-20 methyl glucose is present, it can be in range of from about 1 wt % to about 15 wt % of the modulator component, about 5 wt % to about 10 wt % of the modulator component, less than, equal to, or greater than about 1 wt %, 2, 3, 4, 5, 6, 7, 8, 9, 10, 11, 12, 13, 14, or about 15 wt %.

In some further examples, the modulator component can include other modulators included in addition to pentylene glycol, polycitronellol, or a mixture thereof. Examples of the additional modulators can include those listed herein below in Tables 4 (a) and 4 (b).

Tables 4 (a) and 4 (b) provide lists of suitable non-odorous fragrance modulators.

TABLE 4(a)
Substantially Non-Odorous Fragrance Modulators

| | | | CAS | |
|---|---|---|---|---|
| No. | Group | Chemical Name | Number | Supplier |
| 1. | (a) | PPG-10 Methyl Glucose Ether | 61849-72-7 | Lubrizol |
| 2. | | PPG-20 Methyl | 61849-72-7 | |
| | | Glucose Ether ¹ | | |
| 3. | | Ethoxylated Methyl Glucose | 68239-42-9 | |
| | | Ether ² | | |
| 4. | | Caprylyl/Capryl Glucoside ³ | 68515-73-1 | BASF |
| 5. | | Undecyl Glucoside ^{3a} | — | SEPPIC |
| | | | | (France) |
| 6. | (b) | Isocetyl Alcohol ⁴ | 36653-82-4 | Ashland |
| | | | | Speciality |
| | | | | Ingredients |
| 7. | (c) | PPG-3 Myristyl Ether ⁵ | — | Evonik |
| 8. | | Neopentyl Glycol | 28510-23-8 | Lubrizol |
| | | Diethylhexanoate ⁶ | | |
| 9. | (d) | Sucrose Laurate | 25339-99-5 | Alfa |
| | | | | Chemicals |
| | | | | Ltd. (UK) |
| 10. | | Sucrose dilaurate | 25915-57-5 | Alfa |
| | | | | Chemicals |
| | | | | Ltd. (UK) |
| 11. | | Sucrose Myristate | 27216-47-3 | Mitsubishi |
| | | | | Chemicals |
| 12. | | Sucrose Palmitate | 26446-38-8 | Alfa |
| | | | | Chemicals |
| 13. | | Sucrose Stearate | 25168-73-4 | Ltd. (UK) |
| 14. | | Sucrose Distearate | 27195-16-0 | Mitsubishi |
| | | | | Chemicals |
| | | | | (JP) |
| 15. | | Sucrose Tristearate | 27923063-3 | Mitsubishi |
| | | | | Chemicals |
| | | | | (JP) |
| 16. | (e) | (E)-1-(2,2,6- | — | Takasago |
| | | trimethylcyclohexyl)oct-1- | | (Japan) |
| | | en-3-one ⁸ | | |
| 17. | (f) | 2-(1-menthoxy)ethane-1-ol ⁹ | — | Takasago |
| 18. | | 1-(1-menthoxy)propane-2-ol ⁹ | — | (Japan) |
| 19. | | 3-(1-menthoxy)propane-1-ol ⁹ | — | |
| 20. | | 3-(1-menthoxy)propane-1,2- | — | |
| | | diol ⁹ | | |
| 21. | | 2-methyl-3-(1- | — | |
| | | menthoxy)propane-1,2-diol ⁹ | | |
| 22. | | 4-(1-menthoxy) butane-1-ol ⁹ | — | |
| 23. | (g) | 1,1,4,4-tetramethyl-6-acetyl- | — | Givaudan |
| | | 7-formyl-1,2,3,4- | | (Switzerland) |
| | | tetrahydronaphthalene ¹⁰ | | |
| 24. | | 1,1,2,4,4-pentamethyl-6- | — | |
| | | acetyl-7-formyl-1,2,3,4- | | |
| | | tetrahydronaphthalene ¹⁰ | | |
| 25. | (h) | Hyaluronic acid disaccharide | 9004-61-9 | Sigma |
| | | sodium salt 11 | | Aldrich |
| 26. | | Sodium Hyaluronate ¹¹ | 9067-32-7 | (UK) |
| 27. | (i) | Mono-o-(linalyl)- | — | Kanebo |
| | | glucopyranose ¹² | | (Japan) |
| 28. | | Di-o-(linalyl)- | — | |
| | | glucopyranose ¹² | | |
| 29. | | Tri-o-(linalyl)- | — | |
| | | glucopyranose ¹² | | |
| 30. | | Tetra-o-(linalyl)- | — | |
| | | glucopyranose ¹² | | |
| 31. | | Penta-o-(linalyl)- | — | |
| | | glucopyranose ¹² | | |
| 32. | | Mono-o-(cis-3-hexenyl)- | — | |
| | | glactopyranose ¹² | | |
| 33. | | Di-o-(cis-3-hexenyl)- | — | |
| | | glactopyranose ¹² | | |
| 34. | | Tri-o-(cis-3-hexenyl)- | — | |
| | | glactopyranose ¹² | | |
| 35. | | Tetra-o-(cis-3-hexenyl)- | — | |
| | | glactopyranose ¹² | — | |
| 36. | | Penta-o-(cis-3-hexenyl)- | | |
| | | glactopyranose ¹² | | |
| 37. | (j) | Bis-O-(3,6-dioxadecanyl)- | — | |
| | | glucopyranose ¹³ | | |
| 38. | | Tris-O-(3,6-dioxadecanyl)- | — | |
| | | glucopyranose ¹³ | | |
| 39. | | Tetrakis-O-(3,6-dioxadecanyl)- | — | |
| | | glucopyranose ¹³ | | |
| 40. | | Pentakis-O-(3,6- | — | |
| | | dioxadecanyl)- | | |
| | | glucopyranose¹³ | | |
| 41. | | Bis-O-(3,6-dioxaoctanyl)- | — | |
| | | galactopyranose ¹³ | | |
| 42. | | Tris-O-(3,6-dioxaoctanyl)- | — | |
| | | galactopyranose ¹³ | | |
| 43. | | Tetrakis-O-(3,6-dioxaoctanyl)- | — | |
| | | galactopyranose¹³ | | |
| 44. | | Pentakis-O-(3,6-dioxaoctanyl)- | — | |
| | | galactopyranose ¹³ | | |
| 45. | | Bis-O-(3,6-dioxaheptanyl)- | — | |
| | | xylopyranose ¹³ | | |
| 46. | | Tris-O-(3,6-dioxaheptanyl)- | — | |
| | | xylopyranose ¹³ | | |
| 47. | | Tetrakis-O-(3,6- | — | |
| | | dioxaheptanyl)- | | |
| | | xylopyranose ¹³ | | |
| 48. | | Bis-O-(3,6-dioxadodecanyl)- | — | |
| | | glucopyranose ¹³ | | |
| 49. | | Tris-O-(3,6-dioxadodecanyl)- | — | |
| | | glucopyranose ¹³ | | |
| 50. | | Tetrakis-O-(3,6- | — | |
| | | dioxadodecanyl)- | | |
| | | glucopyranose ¹³ | | |
| 51. | | Pentakis-O-(3,6- | — | |
| | | dioxadodecanyl)- | | |
| | | glucopyranose ¹³ | | |
| 52. | (k) | Hydroquinone beta-D- | 497-76-7 | Shiseido |
| | | glycoside ¹⁴ | | |
| 53. | (l) | Propylene Glycol | 1569-01-3 | Sigma |
| | | Propyl Ether | | Aldrich |
| 54. | | Dicetyl Ether | 4113-12-6 | (UK) |
| 55. | | Polyglycerin-4 Ethers | 25618-55-7 | Solvay |
| | | | | Chemicals |
| 56. | | Isoceteth-5 | 69364-63-2 | Nihon |
| 57. | | Isoceteth-7 | 69364-63-2 | Emulsion |
| 58. | | Isoceteth-10 | 69364-63-2 | Company |
| 59. | | Isoceteth-12 | 69364-63-2 | Ltd. |
| 60. | | Isoceteth-15 | 69364-63-2 | |
| 61. | | Isoceteth-20 | 69364-63-2 | |
| 62. | | Isoceteth-25 | 69364-63-2 | |
| 63. | | Isoceteth-30 | 69364-63-2 | |
| 64. | | Disodium | 68929-04-4 | Rhodia |
| | | Lauroamphodipropionate | | |
| 65. | | Hexaethylene glycol | 3055-96-7 | Sigma |
| | | monododecyl ether ^{14b} | | Aldrich |
| | | | | (UK) |
| 66. | (m) | Neopentyl Glycol | 27841-07-2 | Symrise |
| | | Diisononanoate ¹⁵ | | |
| 67. | | Cetearyl Ethylhexnoate ¹⁶ | 90411-68-0 | (Germany) |
| 68. | (n) | 2-ethylhexyloxy- | 70455-33-9 | Takasago |
| | | propanediol ¹⁷ | | (JP) |
| 69. | (o) | Panthenol Ethyl Ether ¹⁸ | 667-83-4 | DSM |
| | | | | Nutritional |
| | | | | Products, |
| | | | | Inc. (USA) |
| 70. | | DL-Panthenol | 16485-10-2 | Roche Inc. |
| | | | | (USA) |
| 71. | (p) | Diisobutyl Adipate ¹⁹ | 141-04-8 | |
| 72. | | Diisoamyl Adipate ¹⁹ | 6624-70-0 | Sigma |
| | | | | Aldrich |
| | | | | (UK) |
| 73. | (q) | PPG-11 Stearyl Ether ^{19a} | 25231-21-4 | Kao (JP) |
| 74. | (r) | N-hexadecyl n-nonanoate ^{19b} | 72934-15-7 | Symrise |
| | | (e.g., cetyl nonanoate) | | (Germany) |
| 75. | | Noctadecyl n-nonanoate ^{19b} | 107647-13-2 | |
| | | (e.g., stearyl nonanoate) | | |
| 76. | (s) | methanone, (morphonyl) | — | Unilever |
| | | tricyclo[3.3.1.1^{3,7}]dec-1-yl- ²⁰ | | (UK) |
| 77. | | methanone, (piperidinyl) | — | |
| | | tricyclo[3.3.1.1^{3,7}]dec-1-yl- ²⁰ | | |
| 78. | | methanone, (pyrrolidinyl) | — | |
| | | tricyclo[3.3.1.1^{3,7}]dec-1-yl ²⁰ | | |
| 79. | | methanone, (azetidinyl) | — | |
| | | tricyclo[3.3.1.1^{3,7}]dec-1-yl- ²⁰ | | |
| 80. | | methanone, | — | |
| | | (hexahydroazepinyl) | | |
| | | tricyclo[3.3.1.1^{3,7}]dec-1-yl-²⁰ | | |
| 81. | | methanone, (4-cyano- | — | |
| | | piperidinyl)tricyclo | | |
| | | [3.3.1.1^{3,7}]dec-1-yl- ²⁰ | | |
| 82. | | methanone, (4-amido- | — | |
| | | piperidinyl)tricyclo[3.3.1.1^{3,7}] | | |
| | | dec-1-yl- ²⁰ | | |
| 83. | | methanone, | — | |
| | | (Tricyclo[3.3.1.1^{3,7}]decanyl)- | | |
| | | N-tricyclo[3.3.1.1^{3,7}] | | |
| | | dec-1-yl- ²⁰ | | |
| 84. | | methanone, | — | |
| | | (decahydroisoquinolinyl) | | |
| | | tricyclo[3.3.1.1^{3,7}]dec-1-yl- ²⁰ | | |
| 85. | | methanone, | — | |
| | | (decahydroisoquinolinyl) | | |
| | | tricyclo[3.3.1.1^{3,7}]dec-1-yl- ²⁰ | | |
| 86. | | methanone, | — | |
| | | (decahydroquinolinyl)tricyclo | | |
| | | [33.1.1^{3,7}]dec-1-yl-20 | | |
| 87. | | methanone, (3,3-dimethyl-1- | — | |
| | | piperidinyl)tricyclo[3.3.1.1^{3,7}] | | |
| | | dec-1-yl- ²⁰ | | |
| 88. | | methanone, (2-methyl-1- | — | |
| | | piperidinyl)tricyclo[3.3.1.1^{3,7}] | | |
| | | dec-1-yl- ²⁰ | | |
| 89. | | methanone, (4-methyl-1- | — | |
| | | piperidinyl)tricyclo[3.3.1.1^{3,7}] | | |
| | | dec-1-yl- ²⁰ | | |
| 90. | | methanone, (3-methyl-1- | — | |
| | | piperidinyl)tricyclo[3.3.1.1^{3,7}] | | |
| | | dec-1-yl- ²⁰ | | |
| 91. | | methanone, (3,5-dimethyl-1- | — | |
| | | piperidinyl)tricyclo[3.3.1.1^{3,7}] | | |
| | | dec-1-yl- ²⁰ | | |
| 92. | | methanone, (4-methyl-4-ethy- | — | |
| | | piperidinyl)tricyclo[3.3.1.1^{3,7}] | | |
| | | dec-1-yl- ²⁰ | | |
| 93. | | methanone, (3,3-diethyl-1- | — | |
| | | pyrrolidinyl)tricyclo[3.3.1.1^{3,7}] | | |
| | | dec-1-yl- ²⁰ | | |
| 94. | | methanone, (N,N-diisopropyl) | — | |
| | | tricyclo[3.3.1.1^{3,7}]dec-1-yl- ²⁰ | | |
| 95. | | methanone, (3,3- | — | |
| | | dimethylbutylaminyl) | | |
| | | tricyclo[3.3.1.1^{3,7}]dec-1-yl- ²⁰ | | |
| 96. | | methanone, (2,2- | — | |
| | | dimethylpropylaminyl) | | |
| | | tricyclo[3.3.1.1^{3,7}]dec-1-yl- ²⁰ | | |
| 97. | | methanone, (1,1-dimethyl-3,3- | — | |
| | | dimethylbutylaminyl) | | |
| | | tricyclo[3.3.1.1^{3,7}]dec-1-yl- ²⁰ | | |
| 98. | | methanone, (1,3-dimethyl- | — | |
| | | butylaminyl) | | |
| | | tricycle[3.3.1.1^{3,7}]dec-1-yl- ²⁰ | | |
| 99. | (t) | Bis-methoxy PEG-13 PEG- | 936645-35-1 | Polymer- |
| | | 438/PPG-110 SMDI | | Expert |
| | | Copolymer ²¹ | | S.A. |
| | | | | (Pessac, |
| | | | | France) |
| 100. | (u) | propyl {4-[2-(diethylamino)- | 61791-12-6 | Sigma |
| | | 2-oxoethoxy]-3- | | Aldrich |
| | | methoxyphenyl}acetate ²² | | (US) |
| 101. | (v) | 3-((2-ethylhexyl)oxy)propane- | 70445-33-9 | — |
| | | 1,2-diol ²³ | | |
| 102. | | 3-((2- | — | — |
| | | propylheptyl)oxy)propane- | | |
| | | 1,2-diol ²³ | | |
| 103. | | 1-amino-3-((2- | 99509-00-9 | — |
| | | ethylhexyl)oxy)propan-2-ol ²³ | | |

¹ available as GLUCAM ™ P-20.
² available as Glucam ™ E-20.
³ available as Plantacare ® 810 UP.
^{3a} available as Simulsol ® SL 11W.
⁴ available as CERAPHYL ® ICA.
⁵ available as Tegosoft ® APM.
⁶ available as Schercemol ™ NGDO.
⁷ disclosed in U.S. Pat. No. 6,737,396B2 (Firmenich), column 1, lines 43-47.
⁸ diclosed as compound 1'i in U.S. Pat. No. 6,440,400B1 (Takasago Int. Corp.), col. 5.
^{8a} diclosed in U.S. Pat. No. 4,313,855 (Dragoco Gerberding & Co. GmbH), col. 1, lines 12-13.
⁹ disclosed in U.S. Pat. No. 7,538,081B2 (Takasago Int. Corp.), column 7, lines 50-53.
¹⁰ disclosed in U.S. Pat. No. 6,147,049 (Givaudan Roure), col. 5, line 24, to col. 6, line 17.
¹¹ disclosed in PCT Publication No. WO85/04803 (Diagnostic), pg. 2, line 1 to pg. 4, line 2.
¹² disclosed in JP Pat. No. 61-083114 (Kanebo).
¹³ disclosed in JP Pat. No. 61-063612 (Kanebo).
¹⁴ disclosed in JP Pat. No. 62-084010 (Shiseido).
^{14b} available as: Laureth-6.
¹⁵ disclosed in U.S. Patent Publication No. 2011/0104089A1 (Symrise), para. [0001].
¹⁶ available as PCL-Liquid ® 100.
¹⁷ disclosed in U.S. Pat. No. 7,196,052 (Takasago Int. Corp.), col. 4, lines 34-35.
¹⁸ disclosed in EP Patent Publication No. 616800A2 (Givaudan), pg. 2, lines 12-25.
¹⁹ disclosed in U.S. Pat. No. 4,110,626 (Shiseido), column 3, lines 54-56.
^{19a} disclosed in PCT Publication No. WO2014/155019 (LVMH).
^{19b} disclosed in U.S. Pat. No. 9,050,261 (Symrise).
²⁰ disclosed as compounds C1-C22 in WO2014/139952 (Unilever).
²¹ available as Expert Gel ® EG56.
²² available as Kolliphor ® EL.
²³ disclosed in U.S. Pat. No. 9,050,261 (Symrise).

Further examples of non-odorous fragrance modulator is selected from the group of materials disclosed in Table 4(b).

TABLE 4(b)
Substantially Non-Odorous Fragrance Modulators

| | Chemical or INCI | | | |
|---|---|---|---|---|
| No. | Name | Trade Name | CAS Number | Supplier |
| 1. | C12-14 Sec-Pareth-3 | Tergitol ® 15-S- | 68131-40-8 | Sigma Aldrich |
| | | 7 | | (UK) |
| 2. | Poly(ethylene glycol- | PPG-7-Buteth- | 9038-95-3 | Sigma Aldrich |
| | ran-propylene glycol) | 10 | | (UK) |
| | monobutyl ether | | | |
| 3. | PPG-4-Ceteth-10 | Nikkol PBC-33 | 37311-01-6 | Chemical Navi |
| 4. | Deceth-4 | Ethal DA-4 | 5703-94-6 | Ethox Chemicals, |
| | | | | Inc. |
| 5. | PPG-5-Ceteth-20 | AEC PPG-5- | 9087-53-0 | A & E Connock |
| | | Ceteth-20 | | (Perfumery & |
| | | | | Cosmetics) Ltd. |
| 6. | C14-15 Pareth-7 | Neodol 45-7 | 68951-67-7 | Shell Chemical |
| | | alcohol | | Company |
| | | ethoxylate | | |
| 7. | Linear alcohol (C12-15) | Bio-soft N25-7 | 68131-39-5 | Stephan |
| | Pareth-3ethoxylate, | | | Company (USA) |
| | POE-7 | | | |
| 8. | Linear alcohol (C12-13) | Bio-soft N23- | 66455-14-9 | |
| | Pareth- | 6.5 | | |
| | 3ethoxylated, POE-6.5) | | | |
| 9. | Polyethylene glycol | Cremophor ® A | 68439-49-6 | Sigma Aldrich |
| | 1100 | 25 | | (UK) |
| | mono(hexadecyl/ | | | |
| | octadecyl) ether | | | |
| 10. | Linear alcohol (C9-11) | Bio-soft N91-8 | 68439-46-3 | Stephan |
| | ethoxylated POE -8 | | | Company (USA) |
| | Pareth-3 | | | |
| 11. | Coceth-10 or | Genapol ® C- | 61791-13-7 | Sigma Aldrich |
| | Polyoxyethylene (10) | 100 | | (UK) |
| | dodecyl ether | | | |
| 12. | Alcohols, C12-14, | Rhodasurf ® LA | 68439-50-9 | Solvay Solutions |
| | ethoxylated | 30 | | Italia S.p.A. |
| 13. | Poly(ethylene glycol) | Poly(ethylene | 9004-74-4 | Sigma Aldrich |
| | methyl ether | glycol) methyl | | (UK) |
| | | ether | | |
| 14. | C10-16 Pareth-1 | Neodol ® PC | 68002-97-1 | Shell Chemical |
| | | 110 | | Company |
| 15. | PPG-11 Stearyl Ether | Arlamol ™ | 25231-21-4 | Croda (UK) |
| | | PS11E | | |
| 16. | Steareth-100 | Brij ® S100 | 9005-00-9 | Sigma Aldrich |
| | | | | (UK) |
| 17. | Polyethylene glycol | Brij ® C-58 | 9004-95-9 | Sigma Aldrich |
| | hexadecyl ether | | | (UK) |
| 18. | Pluronic ® F-127 | Pluronic ® F-127 | 9003-11-6 | Sigma Aldrich |
| | | | | (UK) |
| 19. | Linear Alcohol (C11) | Bio-soft N1-5 | 34398-01-1 | Stepan Canada |
| | Ethoxylate, POE-5 | | | Inc. |
| 20. | Laureth-10 | Intrasol FA | 6540-99-4 | Evonik Industries |
| | | 12/18/10 | | AG |
| 21. | Decaethylene glycol | Polyoxyethylene | 9002-92-0 | Sigma Aldrich |
| | mono-dodecyl ether | (10) lauryl | | (UK) |
| | | ether | | |
| 22. | Ethylene glycol | 2- | 109-86-4 | Sigma Aldrich |
| | monomethyl ether | Methoxyethanol | | (UK) |
| 23. | Myreth-4 | Homulgator 920 | 27306-79-2 | Grau Aromatics |
| | | G | | GmbH & |
| | | | | Company KG |
| 24. | Oleth-16 Alkoxylated | Pegnol O-16A | 25190-05-0 | Toho Chemical |
| | Alcohols | | | Industry Co., Ltd. |
| 25. | Isosteareth-5 | Emalex 1805 | 52292-17-8 | Nihon Emulsion |
| | | | | Company, Ltd. |
| 26. | PPG-10 Cetyl Ether | Arlamol ™ | 9035-85-2 | Croda (UK) |
| | | PC10 | | |
| 27. | Polyoxy(ethylene | Poly(ethylene | 24938-91-8 | Sigma-Aldrich |
| | glycol) (18) tridecyl | glycol) (18) | | (UK) |
| | ether | tridecyl ether | | |
| 28. | Poly(oxy-1,2- | ALFONIC ® 10- | 26183-52-8 | Sasol Chemicals |
| | ethanediyl), a-decyl-w- | 8 Ethoxylate | | (USA) LLC |
| | hydroxy- | | | |
| 29. | Laureth- 1 | Mackam ™ | 4536-30-5 | Rhodia (DE) |
| | | 2LSF | | |
| 30. | PEG-5 Hydrogenated | Ethox HTAM-5 | 61791-26-2 | Ethox Chemicals, |
| | Tallow Amine | | | Inc. |
| 31. | PEG-15 Oleamine | Nikkol | 26635-93-8 | Nikko Chemicals |
| | | TAMNO-15 | | Co., Ltd. |
| 32. | Polyoxyethylene (20) | Brij ® O20-SS | 9004-98-2 | Sigma Aldrich |
| | oleyl ether | | | (UK) |
| 33. | Cetoleth-10 | Brij ® CO10 | 8065-81-4 | Croda, Inc. |
| 34. | Talloweth-7 | Emulmin 70 | 61791-28-4 | Sanyo Chemical |
| | | | | Industries Ltd. |
| 35. | Isobutoxypropanol | Isobutoxypropanol | 34150-35-1 | MolPort |
| | Alcohols | | | |
| 36. | Isobutoxypropanol | Isobutoxypropanol | 23436-19-3 | AKos Consulting |
| | Alcohols | | | & Solutions |
| 37. | Diethylene Glycol | Twincide EDG | 111-46-6 | Roda |
| 38. | Methoxyethanol | Hisolve MC | 109-86-4 | Toho Chemical |
| | | | | Industry Co., Ltd. |
| 39. | Ethoxyethanol | 2- | 110-80-5 | Sigma-Aldrich |
| | Alcohols | Ethoxyethanol | | (UK) |
| 40. | Methoxyisopropanol | Dowanol ™ PM | 107-98-2 | The Dow |
| | Alcohols | | | Chemical |
| | | | | Company |
| 41. | Methoxyethanol | Hisolve MC | 32718-54-0 | Toho Chemical |
| | | | | Industry Co., Ltd. |
| 42. | Methylal | Dimethoxymethane | 109-87-5 | Sigma-Aldrich |
| | Ethers | | | (UK) |
| 43. | 3-Methoxybutanol | Methoxybutanol | 2517-43-3 | Hans |
| | | | | Schwarzkopf |
| | | | | GmbH/Co. KG |
| 44. | Butoxyethanol | Butyl OXITOL | 111-76-2 | Shell Chemical |
| | | | | Company |
| 45. | Propylene Glycol n- | Dowanol ™ | 5131-66- | The Dow |
| | Butyl Ether | PnB | 8/29387-86-8 | Chemical |
| | | | | Company |
| 46. | Propylene Glycol Butyl | Propylene | 15821-83-7 | Sigma Aldrich |
| | Ether | Glycol Butyl | | (UK) |
| | | Ether | | |
| 47. | 2-(2- | Diethylene | 112-34-5 | Sigma Aldrich |
| | butoxyethoxy)ethanol | glycol butyl | | (UK) |
| | | ether | | |
| 48. | Deceth-4 Phosphate | Crodafos ™ | 52019-36-0 | Croda, Inc. |
| | | D4A | | |
| 49. | 2- | Ethylene glycol | 2136-71-2 | Sigma-Aldrich |
| | (Hexadecyloxy)ethanol | monohexadecyl | | (UK) |
| | | ether | | |
| 50. | Poly(propylene glycol) | Poly(propylene | 9003-13-8 | Sigma-Aldrich |
| | monobutyl ether | glycol) | | (UK) |
| | | monobutyl ether | | |
| 51. | Propylene Glycol | Dowanol ™ PnP | 30136-13-1 | The Dow |
| | Propyl Ether | | | Chemical |
| | | | | Company |
| 52. | Propylene Glycol n- | Dowanol ™ | 29387-86- | The Dow |
| | Butyl Ether | PnB | 8/5131-66-8 | Chemical |
| | | | | Company |
| 53. | Dipropylene glycol | Di(propylene | 34590-94-8 | Sigma Aldrich |
| | monomethyl ether | glycol) methyl | | (UK) |
| | | ether, mixture | | |
| | | of isomers | | |
| 54. | Dipropylene Glycol | Proglyde ™ | 111109-77-4 | The Dow |
| | Dimethyl Ether | DMM | | Chemical |
| | | | | Company |
| 55. | PPG-2 Methyl Ether | Dowanol ™ | 13429-07-7 | The Dow |
| | | DPM | | Chemical |
| | | | | Company |
| 56. | Methoxydiglycol | OriStar | 111-77-3 | Orient Stars LLC |
| | Ethers | DEGME | | |
| 57. | Diethylene glycol ethyl | Di(ethylene | 111-90-0 | Sigma Aldrich |
| | ether | glycol) ethyl | | (UK) |
| | | ether | | |
| 58. | Dimethoxydiglycol | Dimethyldiglycol | 111-96-6 | H&V Chemicals |
| | Ethers | | | |
| 59. | PPG-3 Methyl Ether | Dowanol ™ | 37286-64-9 | The Dow |
| | | TPM | | Chemical |
| | | | | Company |
| 60. | Methyl Morpholine | 224286 | 7529-22-8 | Sigma-Aldrich |
| | Oxide | ALDRICH | | (UK) |
| | Amine Oxides | 4- | | |
| | | Methylmorpholine | | |
| | | N-oxide | | |
| 61. | Oleth-3 | Brij ® O3 | 5274-66-8 | Croda Europe, |
| | | | | Ltd. |
| 62. | Tri(propylene glycol) | Dowanol ™ | 55934-93-5 | Sigma-Aldrich |
| | n-butyl ether | TPnB | | (UK) |
| 63. | Tripropylene Glycol | Tripropylene | 24800-44-0 | Sigma-Aldrich |
| | | Glycol | | (UK) |
| 64. | PPG-3 Methyl Ether | Dowanol ™ | 25498-49-1 | The Dow |
| | Alkoxylated Alcohols | TPM | | Chemical |
| | | | | Company |
| 65. | Triethylene glycol | Triglycol | 112-27-6 | Sigma Aldrich |
| | | | | (UK) |
| 66. | PEG-3 Methyl Ether | Hymol ™ | 112-35-6 | Toho Chemical |
| | | | | Industry Co., Ltd. |
| 67. | Laureth-3 | AEC Laureth-3 | 3055-94-5 | A & E Connock |
| | | | | (Perfumery & |
| | | | | Cosmetics) Ltd. |
| 68. | Ethylhexylglycerin | AG-G-75008 | 70445-33-9 | Angene Chemical |
| 69. | Tetra(ethylene glycol) | Tetraethylene | 112-60-7 | Sigma Aldrich |
| | | glycol | | (UK) |
| 70. | Steareth-3 | Isoxal 5 | 4439-32-1 | Vevy Europe SpA |
| 71. | Ceteth-3 | Emalex 103 | 4484-59-7 | Nihon Emulsion |
| | | | | Company, Ltd. |
| 72. | Myreth-3 | Isoxal 5 | 26826-30-2 | Vevy Europe SpA |
| 73. | Trideceth-3 | Alfonic ® TDA- | — | Sasol North |
| | | 3 Ethoxylate | | America, Inc. |
| 74. | Ceteth-2 | Brij ® C2 | 5274-61-3 | Croda Europe, |
| | | | | Ltd. |
| 75. | Oleth-2 | Brij ® O2 | 5274-65-7 | Croda, Inc. |
| 76. | Steareth-2 | Brij ® S2 | 16057-43-5 | Croda, Inc. |
| 77. | Cetoleth-10 | Brij ® CO10 | 8065-81-4 | Croda, Inc. |
| 78. | Trimethyl Pentanol | Trimethyl | 68959-25-1 | Angene Chemical |
| | Hydroxyethyl Ether | Pentanol | | |
| | Alcohols | Hydroxyethyl | | |
| | | Ether | | |
| 79. | Steareth-10 Allyl Ether | Salcare ® SC80 | 109292-17-3 | BASF |
| 80. | TEA-Lauryl Ether | material ID- | 1733-93-3 | Angene Chemical |
| | | AG-J-99109 | | |
| 81. | Polyglyceryl-2 Oleyl | Chimexane NB | 71032-90-1 | Chimex |
| | Ether | | | |
| 82. | Batyl Alcohol | B402 | 544-62-7 | Sigma-Aldrich |
| | | ALDRICH | | (UK) |
| 83. | Octaethylene Glycol | 15879 | 5117-19-1 | Sigma-Aldrich |
| | | ALDRICH | | (UK) |
| 84. | Triglycerol | Cithrol ™ | 66082-42-6 | Croda (UK) |
| | diisostearate | | | |
| 85. | Diglycerin | Diglycerin 801 | 59113-36-9 | Sakamoto |
| | | | | Yakuhin Kogyo |
| | | | | Co., Ltd. |
| 86. | Polyglycerin #310 | Polyglycerin | 25618-55-7 | Sakamoto |
| | | #310 | | Yakuhin Kogyo |
| | | | | Co., Ltd. |
| 87. | Distearyl Ether | Cosmacol ® SE | 6297-03-6 | Sasol Germany |
| | | | | GmbH |
| 88. | Caprylyl Glyceryl | Caprylyl | 10438-94-5 | AKos Consulting |
| | Ether | Glyceryl Ether | | & Solutions |
| 89. | Chimyl Alcohol | Chimyl Alcohol | 506-03-6 | Nikko Chemicals |
| | | | | Co., Ltd. |
| 90 | Dipentaerythrityl | Liponate ® DPC- | 68130-24-5 | Lipo Chemicals, |
| | Hexacaprylate/Hexacap | 6 | | Inc. |
| | rate | | | |
| 91. | Morpholine | 394467 | 110-91-8 | Sigma-Aldrich |
| | | ALDRICH | | (UK) |
| 92. | Dimethyl Oxazolidine | OXABAN ™ -A | 51200-87-4 | The Dow |
| | | | | Chemical |
| | | | | Company |
| 93. | Ethyl Hydroxymethyl | 4- | 68140-98-7 | Angene Chemical |
| | Oleyl Oxazoline | Oxazolemethanol | | |
| 94. | Methyl Hydroxymethyl | Adeka Nol GE- | 14408-42-5 | Adeka |
| | Oleyl Oxazoline | RF | | Corporation |
| 95. | Pramoxine HCl | OriStar | 637-58-1 | Orient Stars LLC |
| | | PMHCL | | |
| 96. | Allantoin Ascorbate | Allantoin | 57448-83-6 | ABI Chem |
| | | Ascorbate | | |
| 97. | Stearamidopropyl | Mackalene ™ | 55852-14-7 | Rhodia Inc. |
| | Morpholine Lactate | 326 | | |
| 98. | Dioxolane | Elcotal DX | 646-06-0 | Lambiotte & CIE |
| | | | | S.A. |
| 99. | Glycerol Formal | Glycerol | 5464-28-8 | Sigma Aldrich |
| | | Formal | | (UK) |
| 100. | Stearamidopropyl | Mackine 321 | 55852-13-6 | Rhodia Inc. |
| | Morpholine | | | |
| 101. | 2,4,6- | Poly(melamine- | 68002-20-0 | Sigma-Aldrich |
| | Tris[bis(methoxymethyl)amino]- | co- | | (UK) |
| | 1,3,5-triazine | formaldehyde) | | |
| | | methylated | | |
| 102. | Poloxamine 1307 | Pluracare ® 1307 | 11111-34-5 | BASF |
| 103. | Nonoxynol-8 | Igepal ® CO-610 | 27177-05-5 | Rhodia Inc. |
| 104. | Nonoxynol-10 | Igepal ® CO-710 | 27177-08-8 | Rhodia Inc. |
| 105. | Octoxynol-10 | Nikkol OP-10 | 2315-66-4 | Nikko Chemicals |
| | | | | Co., Ltd. |
| 106. | Nonoxynol-9 | Igepal ® CO-630 | 68987-90-6 | Rhodia Inc. |
| 107. | Nonoxynol-9 Iodine | Nonoxynol-9 | 94349-40-3 | Angene Chemical |
| | | iodine | | |
| 108. | Octylphenoxy | Igepal ® CA-630 | 68987-90-6 | Rhodia Inc. |
| | poly(ethyleneoxy)ethanol, | | | |
| | branched | | | |
| 109. | Sodium Octoxynol-2 | Triton ™ X-200 | 55837-16-6 | The Dow |
| | Ethane Sulfonate | | | Chemical |
| | | | | Company |
| 110. | Benzylhemiformal | Preventol D2 | 14548-60-8 | Lanxess |
| | | | | Corporation |
| 111. | Nonoxynol-2 | Igepal ® CO-210 | 27176-93-8 | Rhodia Inc. |
| 112. | Octoxynol-3 | Igepal ® CA-420 | 2315-62-0 | The Dow |
| | | | | Chemical |
| | | | | Company |
| 113. | Nonoxynol-3 | Marlophen NP | 27176-95-0 | Sasol Germany |
| | | 3 | | GmbH |
| 114. | Alkoxylated Alcohols | Alkasurf NP-4 | 7311-27-5 | Rhodia Inc. |
| 115. | Nonoxynol-3 | Triethylene | 51437-95-7 | Santa Cruz |
| | | Glycol Mono(p- | | Biotechnology |
| | | nonylpheny1) | | |
| | | Ether | | |
| 116. | Nonoxynol-7 | Lowenol 2689 | 27177-03-3 | Jos. H. |
| | | | | Lowenstein & |
| | | | | Sons, Inc. |
| 117. | Nonoxynol-6 | Igepal ® CO-530 | 27177-01-1 | Rhodia Inc. |
| 118. | Nonoxynol-5 | Igepal ® CO-520 | 20636-48-0 | Rhodia Inc. |
| 119. | Nonoxynol-5 | Igepal ® CO-520 | 26264-02-8 | Rhodia Inc. |
| 120. | Nonoxynol-4 | Alkasurf NP-4 | 27176-97-2 | Rhodia Inc. |
| 121. | Polyglyceryl-10 | Nikkol | 102051-00-3 | Nikko Chemicals |
| | Trioleate | Decaglyn 3-OV | | Co., Ltd. |
| 122. | Polyglyceryl-10 | Nikkol | 33940-99-7 | Nikko Chemicals |
| | Dioleate | Decaglyn 2-O | | Co., Ltd. |
| 123. | Polyglyceryl-10 | Caprol 10G40 | 34424-98-1 | Abitec |
| | Tetraoleate | | | Corporation |
| 124. | Polyglyceryl-10 | Nikkol | 79777-30-3 | Nikko Chemicals |
| | Stearate | Decaglyn 1-SV | | Co., Ltd. |
| | | EX | | |
| 125. | Polyglyceryl-10 Oleate | S-Face O-1001 | 79665-93-3 | Sakamoto |
| | | P | | Yakuhin Kogyo |
| | | | | Co., Ltd. |
| 126. | Polyglyceryl-10 | Nikkol | 87390-32-7 | Nikko Chemicals |
| | Myristate | Decaglyn 1-MV | | Co., Ltd. |
| | | EX | | |
| 127. | Dermofeel ® G 10 L | Dermofeel ® G | 34406-66-1 | Dr. Straetmans |
| | | 10 L | | |
| 128. | Polyglyceryl-6 Laurate | NIKKOL | 51033-38-6 | Chemical Navi |
| | | Hexaglyn 1-L | | |
| 129. | Polyglyceryl-6 | S-Face IS-601 P | 126928-07-2 | Sakamoto |
| | Isostearate | | | Yakuhin Kogyo |
| | | | | Co., Ltd. |
| 130. | Choleth-10 | Emalex CS-10 | 27321-96-6 | Nihon Emulsion |
| | | | | Company, Ltd. |
| 131. | Steareth-10 Allyl | Salcare ® SC80 | 109292-17-3 | BASF |
| | Ether/Acrylates | | | |
| | Copolymer | | | |
| 132. | Polyvinyl Stearyl Ether | Giovarez ® 1800 | 9003-96-7 | Phoenix |
| | | | | Chemical, Inc. |
| 133. | Dicetyl Ether | Cosmacol Ether | — | Sasol Germany |
| | | 16 | | GmbH |
| 134. | PPG-23-Steareth-34 | Unisafe 34S-23 | 9038-43-1 | Pola Chemical |
| | | | | Industries, Inc. |
| 135. | Stearoxypropyl | Farmin DM E- | 17517-01-0 | Kao Corp. |
| | Dimethylamine | 80 | | |
| 136. | Distearyl Ether | Cosmacol SE | 6297-03-6 | Sasol Germany |
| | | | | GmbH |
| 137. | Polyquaternium-10 | AEC | 55353-19-0 | A & E Connock |
| | | Polyquaternium- | | (Perfumery & |
| | | 10 | | Cosmetics) Ltd. |
| 138. | Octyl ether | Dioctyl ether | 629-82-3 | Sigma Adlrich |
| | | | | (UK) |
| 139. | Ethyl Ether | Diethyl Ether | 60-29-7 | EMD Chemicals |
| 140. | Methyl Hexyl Ether | methyl hexyl | 4747-07-3 | TCI AMERICA |
| | Ethers | ether | | |
| 141. | Ceteth-12 | Emalex 112 | 94159-75-8 | Nihon Emulsion |
| | | | | Company, Ltd. |
| 142. | Ceteth-10 or cetyl | Jeecol CA-10 | 14529-40-9 | Jeen International |
| | alcohol POE-10 | | | |
| 143. | Steareth-10 | Jeecol SA-10 | 13149-86-5 | Jeen International |
| 144. | Nonaethylene glycol | Nonaethylene | 3055-99-0 | Sigma Aldrich |
| | monododecyl ether | glycol | | (UK) |
| | | monododecyl | | |
| | | ether | | |
| 145. | Oleth-10 | Brij ® O10 | 71976-00-6 | Croda, Inc. |
| 146. | Oleth-10 | Brij ® O10 | 24871-34-9 | Croda, Inc. |
| 147. | PEG-12 | Carbowax ™ | 6790-09-6 | The Dow |
| | | PEG 600 | | Chemical |
| | | | | Company |
| 148. | PEG-9 | Sabopeg 400 | 3386-18-3 | Sabo s.p.a. |
| 149. | PEG-10 | DECAETHYL | 5579-66-8 | MolPort |
| | | ENE GLYCOL | | |
| 150. | PEG-6 | Carbowax ™ | 2615-15-8 | The Dow |
| | | PEG 300 | | Chemical |
| | | | | Company |
| 151. | Glycerol propoxylate | Glycerol | 25791-96-2 | Sigma Aldrich |
| | | propoxylate | | (UK) |
| 152. | Glycerol ethoxylate | Glycerol | 31694-55-0 | Sigma Aldrich |
| | | ethoxylate | | (UK) |
| 153. | Laureth-8 | AEC Laureth-8 | 3055-98-9 | A & E Connock |
| | | | | (Perfumery & |
| | | | | Cosmetics) Ltd. |
| 154. | Oleth-8 | Emalex 508 | 27040-03-5 | Nihon Emulsion |
| | | | | Company, Ltd. |
| 155. | Laureth-7 | Alfonic | 3055-97-8 | Sasol North |
| | | 1216CO-7 | | America, Inc. |
| | | Ethoxylate | | |
| 156. | Steareth-7 | Polyoxyethylene | 66146-84-7 | Sigma Aldrich |
| | | (7) stearyl | | |
| | | ether | | |
| 157. | Deceth-6 | Alfonic 1012- | 5168-89-8 | Sasol North |
| | | 6.0 Ethoxylate | | America, Inc. |
| 158. | Steareth-6 | Emalex 606 | 2420-29-3 | Nihon Emulsion |
| | | | | Company, Ltd. |
| 159. | Hexaethylene glycol | Hexaethylene | 3055-96-7 | Sigma-Aldrich |
| | monododecyl ether | glycol | | (UK) |
| | | monododecyl | | |
| | | ether | | |
| 160. | Hexaethylene glycol | Hexaethylene | 5168-91-2 | Sigma-Aldrich |
| | monohexadecyl ether | glycol | | (UK) |
| | | monohexadecyl | | |
| | | ether | | |
| 161. | Beheneth-5 | Nikkol BB-5 | 136207-49-3 | Nikko Chemicals |
| | | | | Co., Ltd. |
| 162. | Myreth-5 | Isoxal 12 | 92669-01-7 | Vevy Europe SpA |
| 163. | Steareth-5 | Jeecol SA-5 | 71093-13-5 | Jeen International |
| | | | | Corporation |
| 164. | Ceteth-5 | Emalex 105 | 4478-97-1 | Nihon Emulsion |
| | | | | Company, Ltd. |
| 165. | Oleth-5 | Brij ® O5 | 5353-27-5 | Croda, Inc. |
| 166. | Laureth-5 | Safol ® 23E5 | 3055-95-6 | Sasol North |
| | | Ethoxylate | | America, Inc. |
| 167. | Steareth-4 | Jeecol SA-4 | 59970-10-4 | Jeen International |
| | | | | Corporation |
| 168. | Laureth-4 | Brij ® L4 | 5274-68-0 | Croda, Inc. |
| 169. | Myreth-4 | Homulgator 920 | 39034-24-7 | Grau Aromatics |
| | | G | | GmbH & |
| | | | | Company KG |
| 170. | Ceteth-4 | Procol CA-4 | 5274-63-5 | Protameen |
| | | | | Chemicals |
| 171. | Oleth-4 | Chemal OA-4 | 5353-26-4 | Chemax, Inc. |
| 172. | Oleth-4 | Chemal OA-4 | 103622-85-1 | Chemax, Inc. |
| 173. | Polyimide-1 | Aquaflex ™ | 497926-97-3 | Chemwill |
| | | XL-30 | | |
| 174. | Polymethoxy Bicyclic | Caswell No. | 56709-13-8 | Angene Chemical |
| | Oxazolidine | 494CA | | |
| 175. | Hydroxymethyl | Zoldine ™ ZT | 6542-37-6 | Angus Chemical |
| | Dioxoazabicyclooctane | | | Company |
| 176. | Dihydro-7a- | 5-Ethyl-1-aza-3,7- | 7747-35-5 | Sigma Aldrich |
| | ethyloxazolo[3,4- | dioxabicyclo[3.3.0] | | (UK) |
| | c]oxazole | octane | | |
| 177. | Dibenzylidene Sorbitol | Disorbene ® | 32647-67-9 | Roquette |
| | | | | America, Inc. |
| 178. | Dimethyldibenzylidene | Millad ® 3988 | 135861-56-2 | Milliken |
| | Sorbitol | | | Chemicals |
| 179. | Laureth-2 | Alfonic | 3055-93-4 | Sasol North |
| | | 1216CO-2 | | America, Inc. |
| | | Ethoxylate | | |
| 180. | 2-(2- | Piperonyl | 51-03-6 | Sigma-Aldrich |
| | Butoxyethoxy)ethyl (6- | Butoxide | | (UK) |
| | propylpiperonyl) ether | | | |
| 181. | Menthone Glycerin | Frescolat ® | 63187-91-7 | Symrise |
| | Acetal | MGA | | |
| 182. | Propylene Glycol | Mackaderm | 68332-79-6 | Rhodia Inc. |
| | Caprylate | PGC | | |
| 183. | Diethoxynonadiene | SBB016951 | 67674-36-6 | Ambinter |
| 184. | Menthoxypropanediol | Coolact ® 10 | 87061-04-9 | Takasago |
| | Alcohols | | | International |
| | | | | Corporation |
| 185. | 2-Diphenylmethoxy- | Diphenhydramine | 147-24-0 | Sigma-Aldrich |
| | N,N- | HCl | | (UK) |
| | dimethylethylamine | | | |
| | hydrochloride | | | |
| 186. | 3-((2- | — | 70445-33-9 | — |
| | ethylhexyl)oxy)propane- | | | |
| | 1,2-diol | | | |
| 187. | 3-((2- | — | — | — |
| | propylheptyl)oxy)propane- | | | |
| | 1,2-diol | | | |
| 188. | 1-amino-3-((2- | — | 99509-00-9 | — |
| | ethylhexyl)oxy)propan- | | | |
| | 2-ol | | | |
| 189. | 1-(1-Methyl-2- | Di(propylene | 29911-27-1 | Sigma Aldrich |
| | propoxyethoxy)-2- | glycol) propyl | | (UK) |
| | propanol | ether | | |

According to some examples, the modulator(s) can be characterized as being “low odor”, “substantially non-odorous”, or non-odorous. In some examples, if the modulator is present at 1 wt % or less, no odor may be detected from the modulator.

In some examples, the fragrance modulator is biodegradable. This can make the fragrance composition to which it is included a “green” or environmentally friendly fragrance composition. Additionally, the stability of a fragrance composition that includes the substantially non-odorous fragrance modulator can be increased. The increase in stability can be shown by observing that the composition undergoes undesirable color formation (e.g., yellowing) at a slower rate.

The fragrance modulator component can be present in an amount of from about 0.1 wt % to about 27 wt % relative to the total weight of the composition of the composition, about 0.5 wt % to about 18 wt %, about 2.5 wt % to about 15 wt %, or less than, equal to, or greater than about 0.1 wt %, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 wt %. If there are more than one fragrance modulators, then the ranges provided hereinabove cover the total of all of the fragrance modulators.

The fragrance modulator can be a liquid at temperatures lower than 100° C., such as at ambient temperature. The fragrance modulators may be fully miscible with the fragrance materials to form a single phase liquid. However, if the fragrance materials are not entirely miscible, or are immiscible, then co-solvents (e.g., dipropylene glycol (DPG), triethyl citrate, or others well known to those skilled in the art) can be added to aid in the solubility of the fragrance materials.

According to various examples, the effect of the fragrance modulator on the fragrance profile, particularly the characters of the fragrance profile which is attributable to the high and moderate volatile fragrance materials, can be improved. By “improved” it is meant that the fragrance profile of the composition, particular the components contributed by at least one of the high and moderate volatile fragrance materials, can be perceived by a panel of experts or professional evaluators or individual experts or professional evaluators at later time points such as, for example, 15 mins, 30 mins, 1 hour, 2 hours, 3 hours, 4 hours, 5 hours, 6 hours, 7 hours, 8 hours, 10 hours, and possibly all the way up to 24 hrs after application as compared to controls, e.g., lacking any of the disclosed non-odorous fragrance modulators such as pentylene glycol, polycitronellol or an equivalent traditional fragrance construction.

Alternatively, by “improved” it can mean that the perception, by a panel of experts or professional evaluators or individual experts or professional evaluators, of the fidelity of the fragrance profile contributed by the high and moderate volatile fragrance materials is markedly increased or enhanced as compared to the controls. “Increased” or “enhanced” means that a panel of experts or professional evaluators or individual experts or professional evaluators perceives the fragrance profile, preferably the characters attributable to the high and/or moderate volatile fragrance materials, of a composition as not changing from its initial impression or the changes are minimal from when the composition was first applied to when it dissipates. In other words, the fidelity of the perceived fragrance profile of the composition is maintained over time. In contrast the composition lacking any of the disclosed nom-odorous fragrance modulators or an equivilant traditional fragrance construction will undergo a rapid loss of the characters attributable to the high and/or moderate volatile fragrance materials.

Such a solution as presented herein provides enhanced or improved fidelity and/or longevity of the fragrance profile, particularly amongst those composition formulated from volatile fragrance materials having moderate to high vapor pressure ranges (greater than or equal to 0.001 Torr (0.000133 kPa) at 25° C.), without having to rely on the presence or significant amounts of the low volatile fragrance materials, which has a tendency to overpower and alter the overall fragrance profile, particularly over time. As a result, the present disclosure provides the perfumer options to formulate compositions having new fragrance profiles not possible before.

Additionally, according to some embodiments, the perceived harshness of overdosing of the fragrance material is mitigated or absent, as compared to the same perception in a fragrance in the absence of the modulator.

### Volatile Solvents

The composition according to the present invention, can include a volatile solvent present in the amount of from about 20 wt % to about 99 wt % relative to the total weight of the composition, about 30 wt % to about 80 wt %, about 55 wt % to about 75 wt %, or less than, equal to, or greater than about 20 wt %, 25, 30, 35, 40, 45, 50, 55, 60, 65, 70, 75, 80, 85, 90, 95, or about 99 wt %, and wherein the solvent is a branch or unbranched C₁ to C₁₀ alkyl, alkenyl or alkynyl group having at least one alcohol moiety, preferably ethanol, or isopropanol, or other alcohols (e.g., methanol, propanol, isopropanol, butanol, and mixtures thereof) commonly found in commercial fine fragrance products.

Accordingly, ethanol may be present in any of the compositions of the present invention, and more specifically, it will form from about 5 wt % to about 95 wt %, or even from about 10 wt % to about 80 wt %, 25 wt % to about 75 wt % of the composition, or combinations thereof, relative to the total weight of the composition. Alternatively, ethanol may be present in an amount of from about 10 wt % or 25 wt % to about 75 wt % or 80 wt %, relative to the total weight of the composition. The ethanol useful in the present invention may be any acceptable quality of ethanol, compatible and safe for the specific intended use of the composition such as, for example, topical applications of fine fragrance or cosmetic compositions.

### Water

In some examples (e.g., those including a volatile solvent), water may be present in any of the compositions of the present invention, and more specifically, it may not exceed about 95 wt % relative to the total weight of the composition, about 90 wt % or less, about 85 wt % or less, about 80 wt % or less, about 75 wt % or less, about 70 wt % or less, about 65 wt % or less, about 60 wt % or less, about 55 wt % or less, about 50 wt % or less, about 45 wt % or less, about 40 wt % or less, about 35 wt % or less, about 30 wt % or less, about 20 wt % or less, about 10 wt %, or less than, equal to, or greater than about 95 wt %, 90, 85, 80, 75, 70, 65, 60, 55, 50, 45, 40, 30, 35, 30, 25, 20, 15, 10, or 5 wt %. Alternatively, water may be present in an amount of from about 5 wt % or about 95 wt % When the composition is a cosmetic composition the level of water should not be so high that the product becomes cloudy thus negatively impacting the product aesthetics. It is understood that the amount of water present in the composition may be from the water present in the volatile solvent (e.g., ethanol) used in the composition, as the case may be.

### Non-Volatile Solvents

The composition may comprise a non-volatile solvent or a mixture of non-volatile solvents. Non-limiting examples of non-volatile solvents include benzyl benzoate, diethyl phthalate, isopropyl myristate, propylene glycol, dipropylene glycol, triethyl citrate, and mixtures thereof. These solvents often are introduced to the product via the perfume oil as many perfume raw materials may be purchased as a dilution in one of these solvents. Where non-volatile solvents are present, introduced either with the perfume materials or separately, then for the purposes of calculating the proportion of fragrance component having a vapor pressure of less than 0.001 Torr (0.000133 kPa) at 25° C. the total fragrance components does not include non-volatile solvents. Where non-volatile solvents are present, introduced either with the perfume materials or separately, then for the purposes of calculating the total level of fragrance component this does not include non-volatile solvents. In addition, if present with cyclic oligosaccharides, the non-volatile solvent may be included at a weight ratio of the non-volatile solvent to the cyclic oligosaccharide of less than 1:1, less than 1:2, less than 1:10, or less than 1:100.

### Entrapment Materials

In other examples, compositions of the present invention can include an entrapment material at a level such that the weight ratio of the entrapment material to the fragrance materials is in the range of from about 1:20 to about 20:1, in some examples, the composition may comprise an entrapment material present in the amount of from about 0.001 wt % to about 40 wt %, from about 0.1 wt % to about 25 wt %, from about 0.3 wt % to about 20 wt %, from about 0.5 wt % to about 10 wt %, or from about 0.75 wt % to about 5 wt %, relative to the total weight of the composition. The compositions disclosed herein may include from 0.001 wt % to 40%, from 0.1 wt % to 25 wt %, from 0.3 wt % to 20 wt %, from 0.5 wt % to 10 wt % or from 0.75 wt % to 5 wt %, relative to the total weight of the composition, of a cyclic oligosaccharide.

Suitable entrapment materials for use herein are selected from polymers; capsules, microcapsules and nanocapsules; liposomes, absorbents; cyclic oligosaccharides and mixtures thereof. Preferred are absorbents and cyclic oligosaccharides and mixtures thereof. Highly preferred are cyclic oligosaccharides (see PCT Publication Nos. WO2000/67721 (Procter & Gamble); and WO2000/67720 (Procter & Gamble); and U.S. Pat. No. 6,893,647 (Procter & Gamble)).

As used herein, the term “cyclic oligosaccharide” means a cyclic structure comprising six or more saccharide units. Preferred for use herein are cyclic oligosaccharides having six, seven or eight saccharide units and mixtures thereof, more preferably six or seven saccharide units and even more preferably seven saccharide units. It is common in the art to abbreviate six, seven and eight membered cyclic oligosaccharides to α, β and γ respectively.

The cyclic oligosaccharide of the compositions used for the present invention may comprise any suitable saccharide or mixtures of saccharides. Examples of suitable saccharides include, but are not limited to, glucose, fructose, mannose, galactose, maltose and mixtures thereof. However, preferred for use herein are cyclic oligosaccharides of glucose. The preferred cyclic oligosaccharides for use herein are α-cyclodextrins or β-cyclodextrins, or mixtures thereof, and the most preferred cyclic oligosaccharides for use herein are β-cyclodextrins.

The cyclic oligosaccharide, or mixture of cyclic oligosaccharides, for use herein may be substituted by any suitable substituent or mixture of substituents. Herein the use of the term “mixture of substituents” means that two or more different suitable substituents can be substituted onto one cyclic oligosaccharide. The derivatives of cyclodextrins consist mainly of molecules wherein some of the OH groups have been substituted. Suitable substituents include, but are not limited to, alkyl groups; hydroxyalkyl groups; dihydroxyalkyl groups; (hydroxyalkyl)alkylenyl bridging groups such as cyclodextrin glycerol ethers; aryl groups; maltosyl groups; allyl groups; benzyl groups; alkanoyl groups; cationic cyclodextrins such as those containing 2-hydroxy-3-(dimethylamino) propyl ether; quaternary ammonium groups; anionic cyclodextrins such as carboxyalkyl groups, sulphobutylether groups, sulphate groups, and succinylates; amphoteric cyclodextrins; and mixtures thereof.

The substituents may be saturated or unsaturated, straight or branched chain. Preferred substituents include saturated and straight chain alkyl groups, hydroxyalkyl groups and mixtures thereof. Preferred alkyl and hydroxyalkyl substituents are selected from C₁-C₈ alkyl or hydroxyalkyl groups or mixtures thereof, more preferred alkyl and hydroxyalkyl substituents are selected from C₁-C₆ alkyl or hydroxyalkyl groups or mixtures thereof, even more preferred alkyl and hydroxyalkyl substituents are selected from C₁-C₄ alkyl or hydroxyalkyl groups and mixtures thereof. Especially preferred alkyl and hydroxyalkyl substituents are propyl, ethyl and methyl, more especially hydroxypropyl and methyl and even more preferably methyl.

Suitable cyclic oligosaccharides for use in the present invention are unsubstituted, or are substituted by only saturated straight chain alkyl, or hydroxyalkyl substituents. Therefore, preferred examples of cyclic oligosaccharides for use herein are α-cyclodextrin, β-cyclodextrin, methyl-α-cyclodextrin, methyl-β-cyclodextrin, hydroxypropyl-α-cyclodextrin and hydroxypropyl-β-cyclodextrin. Most preferred examples of cyclic oligosaccharides for use herein are methyl-α-cyclodextrin and methyl-β-cyclodextrin. These are available from Wacker-Chemie GmbH Hanns-Seidel-Platz 4, Munchen, DE under the tradename Alpha W6 M and Beta W7 M respectively.

The cyclic oligosaccharides of the compositions used for the present invention can be soluble in water, ethanol, or both water and ethanol. As used herein “soluble” means at least about 0.1 g of solute dissolves in 100 mL of solvent, at 25° C. and 1 standard atmospheric pressure (760 mmHg). The cyclic oligosaccharides for use herein have a solubility of at least about 1 g/100 mL, at 25° C. and 1 atm of pressure. In some examples, cyclic oligosaccharides are only present at levels up to their solubility limits in a given composition at room temperature. A person skilled in the art will recognize that the levels of cyclic oligosaccharides used in the present invention will also be dependent on the components of the composition and their levels, for example the solvents used or the exact fragrance oils, or combination of fragrance oils, present in the composition. Therefore, although the limits stated for the entrapment material are preferred, they are not exhaustive.

### Propellants

The compositions described herein may include a propellant. Some examples of propellants include compressed air, nitrogen, inert gases, carbon dioxide, and mixtures thereof. Propellants may also include gaseous hydrocarbons like propane, n-butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 difluoroethane). The propellant may have a concentration from about 15%, 25%, 30%, 32%, 34%, 35%, 36%, 38%, 40%, or 42% to about 70%, 65%, 60%, 54%, 52%, 50%, 48%, 46%, 44%, or 42% by weight of the total fill of materials stored within the container.

### Antiperspirant Active

The compositions described herein may be free of, substantially free of, or may include an antiperspirant active (e.g., any substance, mixture, or other material having antiperspirant activity). Examples of antiperspirant actives include astringent metallic salts, like the inorganic and organic salts of aluminum, zirconium and zinc, as well as mixtures thereof. Such antiperspirant actives include, for example, the aluminum and zirconium salts, such as aluminum halides, aluminum hydroxyhalides, zirconyl oxyhalides, zirconyl hydroxyhalides, and mixtures thereof.

### Other Ingredients

In yet another aspect, the composition consists essentially of the recited ingredients but may contain small amounts (not more than about 10 wt %, preferably no more than 5 wt %, or preferably no more than 2 wt % thereof, relative to the total weight of the composition) of other ingredients that do not impact on the fragrance profile, particularly the evaporation rate and release of the fragrance materials. For example, a fine fragrance composition may comprise stabilizing or anti-oxidant agents, UV filters or quenchers, or colouring agents, commonly used in perfumery. There are a number of other examples of additional ingredients that are suitable for inclusion in the present compositions, particularly in compositions for cosmetic use. These include, but are not limited to, alcohol denaturants such as denatonium benzoate; UV stabilizers such as benzophenone-2; antioxidants such as tocopheryl acetate; preservatives such as phenoxyethanol, benzyl alcohol, methyl paraben, and propyl paraben; dyes; pH adjusting agents such as lactic acid, citric acid, sodium citrate, succinic acid, phosphoric acid, sodium hydroxide, and sodium carbonate; deodorants and anti-microbials such as farnesol and zinc phenolsulphonate; humectants such as glycerine; oils; skin conditioning agents such as allantoin; cooling agents such as trimethyl isopropyl butanamide and menthol; silicones; solvents such as hexylene glycol; hair-hold polymers such as those described in PCT Publication No. WO94/08557 (Procter & Gamble); salts in general, such as potassium acetate and sodium chloride and mixtures thereof.

In yet another aspect, the composition of the present invention, depending on its intended use, is a mixture of fragrance materials possibly together with other ingredients such as, for example, perfume carriers. By the term “perfume carrier”, it is meant to include materials which are practically neutral from a perfumery point of view, e.g., which does not significantly alter the organoleptic properties of perfuming components. The perfume carrier may be a compatible liquid or solid fillers, diluents, and the like. The term “compatible”, as used herein, means that the components of the compositions of this invention are capable of being combined with the primary actives of the present invention, and with each other, in a manner such that there is no interaction which would substantially reduce the efficacy of the composition under ordinary use situations. The type of carrier utilized in the present invention depends on the type of product desired and may comprise, but are not limited to, solutions, aerosols, emulsions (including oil-in-water or water-in-oil), gels, and liposomes. Preferably, the carrier is a liquid and will be a solvent such as, for example, dipropyleneglycol, diethyl phthalate, isopropyl myristate, benzyl benzoate, 2-(2-ethoxyethoxy)-1-ethanol, or ethyl citrate (triethyl citrate).

In yet another aspect, the compositions for use in the present invention may take any form suitable for use, such as for perfumery or cosmetic use. These include, but are not limited to, vapor sprays, aerosols, emulsions, lotions, liquids, creams, gels, sticks, ointments, pastes, mousses, powders, granular products, substrates, cosmetics (e.g., semi-solid or liquid makeup, including foundations) and the like. In some examples, the compositions for use in the present invention take the form of a vapor spray. Compositions of the present invention can be further added as an ingredient to other compositions, preferably fine fragrance or cosmetic compositions, in which they are compatible. As such they can be used within solid composition or applied substrates etc. Examples of products including the composition can include a fabric care product, an air care product, a home care product, a beauty care product, or a mixture thereof. Specific examples of products can include a perfume, an eau de toilette, an eau de parfum, a cologne, a body splash, a lotion, a cream, a shampoo, a conditioner, a hair mist, a body oil, a deodorant, a solid fragrance, or a body spray. The composition can be contacted with skin, hair, or a fabric.

### Article of Manufacture

The composition may be included in an article of manufacture comprising a spray dispenser. The spray dispenser may comprise a vessel for containing the composition to be dispensed. The spray dispenser may comprise an aerosolized composition (e.g., a composition comprising a propellant) within the vessel as well. Other non-limiting examples of spray dispensers include non-aerosol dispensers (e.g., vapor sprays), manually activated dispensers, pump-spray dispensers, or any other suitable spray dispenser available in the art.

### Methods of Using the Compositions

The composition of the present invention according to any embodiments described herein is a useful perfuming composition, which can be advantageously used as consumer products intended to perfume any suitable substrate. As used herein, the term “substrate” means any surface to which the composition of the present invention may be applied to without causing any undue adverse effect. For example, this can include a wide range of surfaces including human or animal skin or hair, paper (fragranced paper), air in a room (air freshener or aromatherapy composition), fabric, furnishings, dishes, hard surfaces and related materials. Preferred substrates include body surfaces such as, for example, hair and skin, most preferably skin.

The composition of the present invention may be used in a conventional manner for fragrancing a substrate. An effective amount of the composition, such as from about 1 μL to about 100 mL, preferably from about 10 μL to about 1,000 μL, more preferably from about 25 μL to about 500 μL, from about 50 μL to about 100 μL, from about 100 μL to about 20 mL, or combinations thereof, is applied to the suitable substrate. Alternatively, an effective amount of the composition of the present invention is less than, equal to, or greater than about 1 μL, 10 μL, 25 μL or 50 μL to about 100 μL, 500 μL, 1,000 μL, 10,000 μL, 10 mL, 20 mL, 25 mL, 30 mL, 40 mL, 50 mL, 60 mL, 70 mL, 80 mL, 90 mL, or 100 mL. The composition may be applied by hand or applied utilizing a delivery apparatus such as, for example, vaporizer or atomizer. Preferably, the composition is allowed to dry after its application to the substrate. The scope of the present invention should be considered to cover one or more distinct applications of the composition or the continuous release of a composition via a vaporizer or other type of atomizer.

The present disclosure provides a method for imparting, intensifying, or modifying an odor on human skin or human hair, comprising applying to human skin and/or human hair the composition of the present invention. Examples of notes or characters that can be enhanced include any of those of: citrus-type note, green-type note, watery-type notes, aromatic-type notes, herbal-type notes, mint-type notes, lavender-type notes, rosemary-type notes, spicy-type notes, cinnamon-type notes, clove-type notes, pepper-type notes, cumin-type notes, ginger-type notes, fougere-type note, patchouli-type notes, floral-type notes, gourmand-type notes, sweet-type notes, vanilla-type notes, amber-type notes, woody-type notes, cedarwood-type notes, sandalwood type notes, vetyver-type notes and mixtures thereof.

Preferably, the fragrance profile or character of the composition of the present invention is detectable by a panel of experts or professional evaluators or individual experts or professional evaluators at later time points such as, for example, 15 mins, 30 mins, 1 hour, 2 hours, 3 hours, 4 hours, 5 hours, 6 hours, 7 hours, 8 hours, 10 hours, and possibly all the way up to 24 hours after application of the composition to a substrate as compared to controls (e.g., those without modulators).

In another aspect, the present invention is also directed to a method of producing a consumer product comprising bringing into contact or mixing into the product an organoleptically active quantity of a composition of the present invention.

### Examples

Various embodiments of the present invention can be better understood by reference to the following Examples which are offered by way of illustration. The present invention is not limited to the Examples given herein.

### Analytical Evaporation Test

### Protocol

The following test is carried out to demonstrate the improved or enhanced longevity of a fragrance profile of a composition vs. a control. In particular, the test measures the effect of a fragrance modulator on the evaporation rate of one or more fragrance materials formulated in a composition. The evaporation response of the fragrance materials to the modulator, as a function of time, is measured using gas chromatography (“GC”).
1. A test composition may comprise a fragrance modulator with either: (i) a fragrance material or (ii) a blend of fragrance materials. The test compositions also contain ethanol, and deionized water. All the ingredients are admixed until evenly distributed in the test compositions.
2. A control composition to the test composition described in 1 above, is made in a similar manner to Step 1, except that the modulator is replaced by Glucam™ P-20 (alternatively referred to a “glucam” a propoxylated methyl glucose ether) or water.
3. An internal standard is needed to correct for variations of the amount of composition dispensed in the evaporation test, as well as loss during the GC analysis. The type of internal standard, its concentration or the step at which it is added to the fragrance material or compositions (test and control) must be selected appropriately by someone skilled in the art. Suitable non-limiting examples of internal standards are triethyl citrate or denatonium benzoate. The resultant solution is used in subsequent steps.
4. A hotplate is set to a temperature of 32° C. An aluminum container, such as TA Instruments T-Zero™ pan, is placed on the hotplate. 20 μL of the test or control composition is introduced in the aluminum container using a micropipette. Alternatively, the aluminum container may be filled with the test or control composition to its full capacity. The time at which this takes place is determined to be time zero (i.e., T=0). Multiple aluminum containers are prepared and left at the set temperature for pre-determined periods of time, such as for example 15 mins, 30 mins, 1 hr, 2 hrs, 3 hrs, 4 hrs, 5 hrs, 6 hrs, 8 hrs and up to 12 hrs.
5. The aluminum container is removed from the hotplate at the end of the pre-determined time and transferred by being inserted into a 4 mL glass vial already containing at least 2 mL of highly volatile solvent, such as high purity ethanol or hexane.
6. The glass vial is mixed to extract the fragrance materials into the solvent phase. 1 mL of the resultant solution is transferred to a 2 mL GC vial.
7. The GC vial is analysed on an Agilent GC system 7890 equipped with an autosampler, or equivalent. A GC column such as DB-5 MS, DB-1 MS models or equivalent phases, is used. Gas chromatography with flame ionization detection (“FID”) or with mass spectrometry (“MS”) can be used for the identification and quantification of fragrance material in the compositions. The column dimensions as well as GC settings, FID gas flows and temperature or MS parameters, must be adjusted to optimize the detection and response of the fragrance material and internal standard being monitored
8. The peak area of the fragrance material and internal standard are recorded. The peak area ratio of the fragrance material and the internal standard is calculated at each time point for each sample composition. The % of non-evaporated fragrance material remaining from T=0 is calculated at each time point for each sample composition. This is done for both the test and control compositions. Significance is determined by comparison of the evaporation profile for the same fragrance material or same fragrance mixture in the test and control compositions.

The constituents of the test compositions are shown in Table 6. The compositions referred to in Table 6 as “glucam”, “citropol”, and “hydrolyte 5” include performance data in Tables 7-16.

### Results

Tables 7-11 show the amount of a fragrance material in a fragrance mixture (comprising at least dihydro mycenol, linalool, dimethyl benzyl carbinol, alpha-terpineol, and carvone) retained after various time points (0 minutes, 30 minutes, 60 minutes, 180 minutes, and 360 minutes), in a Glucam™ P20 control composition and a pentylene glycol (“hydrolite 5”) test composition, respectively.

TABLE 7
Dihydro Myrcenol
CAS
18479-58-8

| | Glucam P20 | Hydrolite 5 |
|---|---|---|
| | Average (n = 3) | (n = 1) |
| Time (min) | % Amount | % Amount |
| 0 | 100.0 | 100.0 |
| 30 | 55.7 | 76.0 |
| 60 | 33.5 | 48.1 |
| 180 | 11.1 | 8.2 |
| 360 | 0.3 | 5.0 |

TABLE 8
Linalool
CAS
78-70-6

| | Glucam P20 | |
|---|---|---|
| | Average | Hydrolite 5 |
| | (n = 3) | (n = 1) |
| Time (min) | % Amount | % Amount |
| 0 | 100.0 | 100.0 |
| 30 | 54.6 | 70.1 |
| 60 | 38.1 | 45.9 |
| 180 | 13.1 | 6.9 |
| 360 | 0.6 | 4.0 |

TABLE 9
Dimethyl
Benzyl Carbinol
CAS
100-86-7

| | Glucam P20 | |
|---|---|---|
| | Average | Hydrolite 5 |
| | (n = 3) | (n = 1) |
| Time (min) | % Amount | % Amount |
| 0 | 100.0 | 100.0 |
| 30 | 80.5 | 93.2 |
| 60 | 61.6 | 64.0 |
| 180 | 35.8 | 20.5 |
| 360 | 8.2 | 13.2 |

TABLE 10
CAS
98-55-5

| | Glucam P20 | |
|---|---|---|
| | Average | Hydrolite 5 |
| | (n = 3) | (n = 1) |
| Time (min) | % Amount | % Amount |
| 0 | 100.0 | 100.0 |
| 30 | 77.1 | 85.6 |
| 60 | 63.7 | 67.3 |
| 180 | 35.3 | 26.6 |
| 360 | 7.2 | 18.6 |

TABLE 11
CAS
2244-16-8

| | Glucam P20 | |
|---|---|---|
| | Average | Hydrolite 5 |
| | (n = 3) | (n = 1) |
| Time (min) | % Amount | % Amount |
| 0 | 100.0 | 100.0 |
| 30 | 72.3 | 81.7 |
| 60 | 52.7 | 48.9 |
| 180 | 26.0 | 8.4 |
| 360 | 1.4 | 5.0 |

Tables 12-16 show the amount of certain fragrance materials in a fragrance mixture (containing at least dihydro mycenol, linalool, dimethyl benzyl carbinol, alpha-terpineol, and carvone) retained after various time points (0 minutes, 30 minutes, 60 minutes, 120 minutes, 180 minutes, and 360 minutes), in a Glucam™ P20 control composition and a Citropol F test composition, respectively.

TABLE 12
Dihydro Myrcenol
CAS
18479-58-8

| | Glucam P20 | Citropol F |
|---|---|---|
| | Average | Average |
| | (n = 3) | (n = 3) |
| Time (min) | % Amount | % Amount |
| 0 | 100.0 | 100.0 |
| 30 | 55.7 | 69.6 |
| 60 | 33.5 | 63.1 |
| 180 | 11.1 | 27.8 |
| 360 | 0.3 | 18.7 |

TABLE 13
Linalool
CAS
78-70-6

| | Glucam P20 | Citropol F |
|---|---|---|
| | Average | Average |
| | (n = 3) | (n = 3) |
| Time (min) | % Amount | % Amount |
| 0 | 100.0 | 100.0 |
| 30 | 54.6 | 70.0 |
| 60 | 38.1 | 66.8 |
| 180 | 13.1 | 32.4 |
| 360 | 0.6 | 16.6 |

TABLE 14
Dimethyl
Benzyl Carbinol
CAS
100-86-7

| | Glucam P20 | Citropol F |
|---|---|---|
| | Average | Average |
| | (n = 3) | (n = 3) |
| Time (min) | % Amount | % Amount |
| 0 | 100.0 | 100.0 |
| 30 | 80.5 | 85.8 |
| 60 | 61.6 | 88.4 |
| 180 | 35.8 | 55.6 |
| 360 | 8.2 | 49.6 |

TABLE 15
Alpha-
Terpineol
CAS
98-55-5

| | Glucam P20 | Citropol F |
|---|---|---|
| | Average | Average |
| | n = 3) | (n = 3) |
| Time (min) | % Amount | % Amount |
| 0 | 100.0 | 100.0 |
| 30 | 77.1 | 82.3 |
| 60 | 63.7 | 84.1 |
| 180 | 35.3 | 59.5 |
| 360 | 7.2 | 56.0 |

TABLE 16
CAS
2244-16-8

| | Glucam P20 | Citropol F |
|---|---|---|
| | Average | Average |
| Carvone | (n = 3) | (n = 3) |
| Time (min) | % Amount | % Amount |
| 0 | 100.0 | 100.0 |
| 30 | 72.3 | 79.8 |
| 60 | 52.7 | 77.7 |
| 180 | 26.0 | 48.1 |
| 360 | 1.4 | 42.8 |

The results show that the Citropol F and Hydrolite 5 modulators can help to retain a fragrance composition longer than a glucam modulator.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Aspects

The following exemplary aspects are provided, the numbering of which is not to be construed as designating levels of importance:

Aspect 1 provides a fragrance composition comprising:
a modulator component in a range of from about 0.1 wt % to about 30 wt % of the fragrance composition; and
a fragrance component present in an amount in a range of from about 0.04 wt % to about 40 wt % of the fragrance composition, the fragrance component comprising at least one of:
   at least one low volatile fragrance material having a vapor pressure less than 0.001 Torr (0.000133 kPa) at 25° C.;
   at least one moderate volatile fragrance material having a vapor pressure in the range of 0.1 Torr to 0.001 Torr (0.0133 kPa to 0.000133 kPa) at 25° C.; and
   at least one high volatile fragrance material having a vapor pressure greater than 0.1 Torr (0.0133 kPa) at 25° C.

Aspect 2 provides the fragrance composition of Aspect 1, wherein the modulator component is present in a range of from about 0.1 wt % to about 27 wt %.

Aspect 3 provides the fragrance composition of any one of Aspects 1 or 2, wherein the modulator component comprises:
the compound according to Formula I:

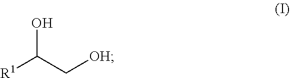

a repeating unit derived from the compound according to Formula II:

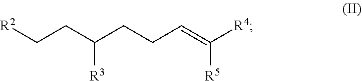

or
a mixture thereof, wherein
R¹, R², R³, R⁴, and R⁵, are independently chosen from —H, —OH, or substituted or unsubstituted (C₁-C₂₀) hydrocarbyl.

Aspect 4 provides the fragrance composition of Aspect 3, wherein R¹, R², R³, R⁴, and R⁵, are independently chosen from (C₁-C₂₀)alkyl, (C₂-C₂₀)alkenyl, (C₃-C₂₀)cycloalkyl, (C₁-C₂₀)alkoxyl, (C₁-C₂₀)aryl, or a combination thereof.

Aspect 5 provides the fragrance composition of any one of Aspects 1-4, wherein the modulator component comprises pentylene glycol, polycitronellol, or a mixture thereof.

Aspect 6 provides the fragrance composition of Aspect 5, wherein the modulator component comprises a mixture of pentylene glycol and polycitronellol and a molar ratio of pentylene glycol to polycitronellol is in a range of from about 5:1 to about 1:5.

Aspect 7 provides the fragrance composition of any one of Aspects 5 or 6, wherein the modulator component comprises a mixture of pentylene glycol and polycitronellol and a molar ratio of pentylene glycol to polycitronellol is in a range of from about 2:1 to about 1:2.

Aspect 8 provides the fragrance composition of any one of Aspects 5-7, wherein the modulator component further comprises about 1 wt % to about 15 wt % PPG-20 methyl glucose ether.

Aspect 9 provides the fragrance composition of any one of Aspects 1-8, wherein the modulator component is free of PPG-20 methyl glucose ether.

Aspect 10 provides the fragrance composition of any one of Aspects 1-9, wherein the modulator component is substantially non-odorous.

Aspect 11 provides the fragrance composition of any one of Aspects 1-10, wherein a pH of the fragrance composition is in a range of from about 4 to about 8.

Aspect 12 provides the fragrance composition of any one of Aspects 1-11, wherein a pH of the fragrance composition is in a range of from about 5 to about 7.

Aspect 13 provides the fragrance composition of any one of Aspects 1-12, wherein the modulator component comprises pentylene glycol.

Aspect 14 provides the fragrance composition of any one of Aspects 1-13, wherein the modulator component comprises polycitronellol.

Aspect 15 provides the fragrance composition of any one of Aspects 1-14, wherein the fragrance composition comprises less than about 78 wt % ethanol.

Aspect 16 provides the fragrance composition of any one of Aspects 1-15, wherein the fragrance composition comprises less than about 50 wt % ethanol.

Aspect 17 provides the fragrance composition of any one of Aspects 1-16, wherein
at least one low volatile fragrance material is present in a range of from about 0 wt % to about 30 wt % of the fragrance component;
at least one moderate volatile fragrance material is present in a range of from about 30 wt % to about 70 wt % of the fragrance component; and
at least one high volatile fragrance material is present in a range of from about 0 wt % to about 30 wt % of the fragrance component.

Aspect 18 provides the fragrance composition of Aspect 17, wherein the at least one high volatile fragrance material is present in an amount of from 10 wt % to about 20 wt % relative to the total weight of the fragrance material.

Aspect 19 provides the fragrance composition of any one of Aspects 17 or 18, wherein the at least one moderate volatile fragrance material is present in an amount of from about 40 wt % to about 60 wt % relative to the total weight of the fragrance material.

Aspect 20 provides the fragrance composition of any one of Aspects 17-19, wherein the at least one low volatile fragrance material is present in an amount of from about 10 wt % to about 20 wt % relative to the total weight of the fragrance material.

Aspect 21 provides the fragrance composition of any one of Aspects 1-20, wherein
at least one low volatile fragrance material is present in a range of from about 30 wt % to about 70 wt % of the fragrance component;
at least one moderate volatile fragrance material is present at greater than about 30 wt % of the fragrance component; and
at least one high volatile fragrance material is present in a range of from about 0.1 wt % to about 30 wt % of the fragrance component.

Aspect 22 provides the fragrance composition of Aspect 21, wherein the at least one low volatile fragrance material is present in an amount of from 31 wt % to about 60 wt % relative to the total weight of the fragrance material.

Aspect 23 provides the fragrance composition of any one of Aspects 21 or 22, wherein the at least one moderate volatile fragrance material is present in an amount of from about 35 wt % to about 60 wt % relative to the total weight of the fragrance material.

Aspect 24 provides the fragrance composition of any one of Aspects 21-23, wherein the at least one high volatile fragrance material is present in an amount of from about 1 wt % to about 30 wt % relative to the total weight of the fragrance material.

Aspect 25 provides the fragrance composition of any one of Aspects 1-24, wherein
at least one low volatile fragrance material is present in a range of from about 0.1 wt % to about 30 wt % of the fragrance component;
at least one moderate volatile fragrance material is present in a range of from about 30 wt % to about 70 wt % of the fragrance component; and
at least one high volatile fragrance material is present at greater than about 30 wt % of the fragrance component.

Aspect 26 provides the fragrance composition of Aspect 25, wherein the at least one high volatile fragrance material is present in an amount of from 31 wt % to about 60 wt % relative to the total weight of the fragrance material.

Aspect 27 provides the fragrance composition of any one of Aspects 25 or 26, wherein the at least one moderate volatile fragrance material is present in an amount of from about 35 wt % to about 60 wt % relative to the total weight of the fragrance material.

Aspect 28 provides the fragrance composition of any one of Aspects 25-27, wherein the at least one low volatile fragrance material is present in an amount of from about 1 wt % to about 30 wt % relative to the total weight of the fragrance material.

Aspect 29 provides the fragrance composition of any one of Aspects 1-28, wherein
at least one low volatile fragrance material is present in a range of from about 30 wt % to about 35 wt % of the fragrance component;
at least one moderate volatile fragrance material is present in a range of from about 30 wt % to about 35 wt % of the fragrance component; and
at least one high volatile fragrance material is present in a range of from about 30 wt % to about 35 wt % of the fragrance component.

Aspect 30 provides the fragrance composition of Aspect 29, wherein the at least one high volatile fragrance material is present in an amount of from about 32 wt % to about 34 wt % relative to the total weight of the fragrance material.

Aspect 31 provides the fragrance composition of any one of Aspects 29 or 30, wherein the at least one moderate volatile fragrance material is present in an amount of from about 32 wt % to about 34 wt % relative to the total weight of the fragrance material.

Aspect 32 provides the fragrance composition of any one of Aspects 29-31, wherein the at least one low volatile fragrance material is present in an amount of from about 32 wt % to about 34 wt % relative to the total weight of the fragrance material.

Aspect 33 provides the fragrance composition of any one of Aspects 1-32, wherein
at least one low volatile fragrance material is present in a range of from about 0 wt % to about 35 wt % of the fragrance component;
at least one moderate volatile fragrance material is present in a range of from about 40 wt % to about 60 wt % of the fragrance component; and
at least one high volatile fragrance material is present in a range of from about 0 wt % to about 30 wt % of the fragrance component.

Aspect 34 provides the fragrance composition of any one of Aspects 1-32, wherein
at least one low volatile fragrance material is present in a range of from about 25 wt % to about 50 wt % of the fragrance component;
at least one moderate volatile fragrance material is present in a range of from about 0 wt % to about 35 wt % of the fragrance component; and
at least one high volatile fragrance material is present in a range of from about 0.1 wt % to about 35 wt % of the fragrance component.

Aspect 35 provides the fragrance composition of any one of Aspects 1-32, wherein
at least one low volatile fragrance material is present in a range of from about 25 wt % to about 60 wt % of the fragrance component;
at least one moderate volatile fragrance material is present in a range of from about 0 wt % to about 35 wt % of the fragrance component; and
at least one high volatile fragrance material is present in a range of from about 25 wt % to about 60 wt % of the fragrance component.

Aspect 36 provides the fragrance composition of any one of Aspects 1-32, wherein
at least one low volatile fragrance material is present in a range of from about 25 wt % to about 60 wt % of the fragrance component;
at least one moderate volatile fragrance material is present in a range of from about 25 wt % to about 60 wt % of the fragrance component; and
at least one high volatile fragrance material is present in a range of from about 25 wt % to about 60 wt % of the fragrance component

Aspect 37 provides the fragrance composition of any one of Aspects 1-36, wherein
the high volatile fragrance material is chosen from any of the materials or combinations of materials listed in any one of Tables 3A and 3B;
the low volatile fragrance material is chosen from chosen from any of the materials or combinations of materials listed in any one of Tables 1A and 1B; and
the moderate volatile fragrance material is chosen from chosen from any of the materials or combinations of materials listed in any one of Tables 2A and 2B.

Aspect 38 provides the fragrance composition of any one of Aspects 1-37, further comprising a carrier.

Aspect 39 provides the fragrance composition of Aspect 38, wherein the carrier is an aqueous carrier or an organic carrier.

Aspect 40 provides the fragrance composition of any one of Aspects 38 or 39, wherein the aqueous carrier is water.

Aspect 41 provides the fragrance composition of any one of Aspects 38 or 40, wherein the organic carrier is ethanol, dipropylene glycol, benzyl benzoate, diethyl phthalate, isopropyl myristate, (C₁-C₂₀)alkyl, (C₁-C₂₀)alkenyl, (C₁-C₂₀)alkynyl, (C₁-C₂₀) hydroxyl, or mixtures thereof.

Aspect 42 provides the fragrance composition of any one of Aspects 38-41, further comprising; a chelator, a thickener, an antioxidant, an emulsifier, or a combination thereof.

1. A fragrance composition comprising:
a modulator component in a range of from about 0.1 wt % to about 30 wt % of the fragrance composition; and
a fragrance component present in an amount in a range of from about 0.04 wt % to about 40 wt % of the fragrance composition, the fragrance component comprising at least one of:
at least one low volatile fragrance material having a vapor pressure less than 0.001 Torr (0.000133 kPa) at 25° C.;
at least one moderate volatile fragrance material having a vapor pressure in the range of 0.1 Torr to 0.001 Torr (0.0133 kPa to 0.000133 kPa) at 25° C.; and
at least one high volatile fragrance material having a vapor pressure greater than 0.1 Torr (0.0133 kPa) at 25° C.

2. The fragrance composition of claim 1, wherein the modulator component is present in a range of from about 0.1 wt % to about 27 wt %.

3. The fragrance composition of any one of claim 1, wherein the modulator component comprises:
the compound according to Formula I: 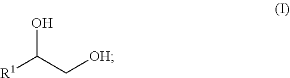
a repeating unit derived from the compound according to Formula II: 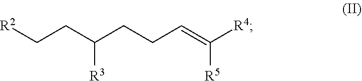
or
a mixture thereof, wherein R¹, R², R³, R⁴, and R⁵, are independently chosen from —H, —OH, or substituted or unsubstituted (C₁-C₂₀) hydrocarbyl.

4. The fragrance composition of claim 1, wherein the modulator component comprises pentylene glycol, polycitronellol, or a mixture thereof.

5. The fragrance composition of claim 4, wherein the modulator component comprises a mixture of pentylene glycol and polycitronellol and a molar ratio of pentylene glycol to polycitronellol is in a range of from about 5:1 to about 1:5.

6. The fragrance composition of claim 1, wherein the modulator component is substantially non-odorous.

7. The fragrance composition of claim 1, wherein a pH of the fragrance composition is in a range of from about 4 to about 8.

8. The fragrance composition of claim 1, wherein the modulator component comprises pentylene glycol or polycitronellol.

9. The fragrance composition of claim 1, wherein
at least one low volatile fragrance material is present in a range of from about 0 wt % to about 30 wt % of the fragrance component;
at least one moderate volatile fragrance material is present in a range of from about 30 wt % to about 70 wt % of the fragrance component; and
at least one high volatile fragrance material is present in a range of from about 0 wt % to about 30 wt % of the fragrance component.

10. The fragrance composition of claim 1, wherein
at least one low volatile fragrance material is present in a range of from about 30 wt % to about 70 wt % of the fragrance component;
at least one moderate volatile fragrance material is present at greater than about 30 wt % of the fragrance component; and
at least one high volatile fragrance material is present in a range of from about 0.1 wt % to about 30 wt % of the fragrance component.

11. The fragrance composition of claim 1, wherein
at least one low volatile fragrance material is present in a range of from about 0.1 wt % to about 30 wt % of the fragrance component;
at least one moderate volatile fragrance material is present in a range of from about 30 wt % to about 70 wt % of the fragrance component; and
at least one high volatile fragrance material is present at greater than about 30 wt % of the fragrance component.

12. The fragrance composition of claim 1, wherein
at least one low volatile fragrance material is present in a range of from about 30 wt % to about 35 wt % of the fragrance component;
at least one moderate volatile fragrance material is present in a range of from about 30 wt % to about 35 wt % of the fragrance component; and
at least one high volatile fragrance material is present in a range of from about 30 wt % to about 35 wt % of the fragrance component.

13. The fragrance composition of claim 1, wherein
at least one low volatile fragrance material is present in a range of from about 0 wt % to about 35 wt % of the fragrance component;
at least one moderate volatile fragrance material is present in a range of from about 40 wt % to about 60 wt % of the fragrance component; and
at least one high volatile fragrance material is present in a range of from about 0 wt % to about 30 wt % of the fragrance component.

14. The fragrance composition of claim 1, wherein
at least one low volatile fragrance material is present in a range of from about 25 wt % to about 50 wt % of the fragrance component;
at least one moderate volatile fragrance material is present in a range of from about 0 wt % to about 35 wt % of the fragrance component; and
at least one high volatile fragrance material is present in a range of from about 0.1 wt % to about 35 wt % of the fragrance component.

15. The fragrance composition of claim 1, wherein
at least one low volatile fragrance material is present in a range of from about 25 wt % to about 60 wt % of the fragrance component;
at least one moderate volatile fragrance material is present in a range of from about 0 wt % to about 35 wt % of the fragrance component; and
at least one high volatile fragrance material is present in a range of from about 25 wt % to about 60 wt % of the fragrance component.

16. The fragrance composition of claim 1, wherein
at least one low volatile fragrance material is present in a range of from about 25 wt % to about 60 wt % of the fragrance component;
at least one moderate volatile fragrance material is present in a range of from about 25 wt % to about 60 wt % of the fragrance component; and
at least one high volatile fragrance material is present in a range of from about 25 wt % to about 60 wt % of the fragrance component

17. The fragrance composition of claim 1, wherein
the high volatile fragrance material is chosen from any of the materials or combinations of materials listed in any one of Tables 3A and 3B;
the low volatile fragrance material is chosen from chosen from any of the materials or combinations of materials listed in any one of Tables 1A and 1B; and
the moderate volatile fragrance material is chosen from chosen from any of the materials or combinations of materials listed in any one of Tables 2A and 2B.

18. The fragrance composition of claim 1, further comprising a carrier.

19. The fragrance composition of claim 18, wherein the carrier is an aqueous carrier or an organic carrier.

20. The fragrance composition of claim 18, wherein the organic carrier is ethanol, dipropylene glycol, benzyl benzoate, diethyl phthalate, isopropyl myristate, (C₁-C₂₀)alkyl, (C₁-C₂₀)alkenyl, (C₁-C₂₀)alkynyl, (C₁-C₂₀) hydroxyl, or mixtures thereof.
